(19) United States
(12) Patent Application Publication
(10) Pub. No.: US 2013/0045323 A1
(43) Pub. Date: Feb. 21, 2013

(54) METHODS OF INCREASING PROTEIN, OIL, AND/OR AMINO ACID CONTENT IN A PLANT

(71) Applicant: Heiko Härtel, Berlin (DE)

(72) Inventor: Heiko Härtel, Berlin (DE)

(73) Assignee: BASF PLANT SCIENCE COMPANY GMBH, LUDWIGSHAFEN (DE)

(21) Appl. No.: 13/587,041

(22) Filed: Aug. 16, 2012

Related U.S. Application Data
(60) Provisional application No. 61/525,225, filed on Aug. 19, 2011.

(51) Int. Cl. A01H 5/00; C12N 5/10; A01H 5/10; C12N 15/82; A01H 1/06; A01H 1/02; C12N 1/21; C12N 1/15; C12N 1/13; C12N 1/19; A01G 1/00; A01C 11/00; A01D 45/00; A23K 1/00; A23L 1/10; A23K 1/18; A23L 1/305; A23L 1/307; A23K 1/16; A23J 1/12; C12N 15/63

(52) U.S. Cl. 426/623; 435/320.1; 435/419; 800/298; 435/468; 800/281; 800/278; 800/275; 800/320.1; 800/264; 435/252.31; 435/252.33; 435/252.32; 435/252.3; 435/252.2; 435/254.8; 435/257.2; 435/254.3; 435/254.6; 435/254.11; 435/254.4; 435/254.7; 435/254.2; 435/254.22; 435/254.23; 435/254.21; 47/58.1 R; 426/627; 426/665

(57) ABSTRACT

The present invention relates to methods for increasing the protein, oil, and/or amino acid content of a plant. The methods involve the manipulation of the expression level of trehalose-6-phosphate synthase (TPS) homologs. Expression cassettes for achieving such gene expression manipulation and transgenic plant cells and plants comprising the constructs and cassettes are also provided. Methods of plant breeding using these plants have also been developed.

### CROSS REFERENCE TO RELATED APPLICATIONS

This application claims the priority benefit of U.S. Provisional Application Ser. No. 61/525,225 filed Aug. 19, 2011 the entire content of which is hereby incorporated by reference in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_List_{—}17731_{—}00040_US. The size of the text file is 277 KB, and the text file was created on Jul. 13, 2012.

### FIELD OF THE INVENTION

This invention relates generally to methods for increasing protein, oil, and/or amino acid content in a plant, plant cell or plant part relative to a corresponding wild-type plant, plant cell or plant part by manipulating the expression of a trehalose-6-phosphate synthase (TPS) homolog. Expression cassettes for achieving such gene expression manipulation, as well as recombinant constructs, vectors and plants, plant cells, or plant parts comprising the same, are also provided. Plants, plant cells, or plant parts with increased content in one or more of protein, oil, or one or more amino acids thus obtained may be useful in the preparation of foodstuffs and animal feeds. Plants, plant cells, or plant parts with increased content in one or more of protein, oil, or one or more amino acids thus obtained may also be useful in plant breeding programs for developing further hybrid or inbred lines.

### BACKGROUND OF THE INVENTION

Crops such as rice, corn, grain sorghum, wheat, oats, rye, and barley are a major source of animal feed for many types of livestock and supply most of their dietary needs. These crops are also a primary source for human food and other industrial purposes. Corn tends to be the preferred feed grain because of its highly digestible carbohydrate content and relatively low fiber content, which is particularly important for swine and poultry (Hard, Proc. Southwest Nutr. Conf., 2005, 43-54). As a result, corn is the most widely produced feed grain globally, accounting for more than 90% of the grain used in feed. However, corn, as well as other crops commonly used as feed grain, have nutritional limitations such as protein and/or oil content, amino acid composition, minerals and vitamins for several types of livestock, especially swine, poultry, and cattle.

Because of the suboptimal protein and/or oil content and amino acid composition of plants in comparison to the nutritional requirement of the animal, it is common practice to use feed additives and supplements in animals diets. These feed additives and supplements include protein-rich feeds, amino acids, vitamins, minerals and fats. The nutritional limitations of feed grain have become more critical as the demand for higher feeding efficiency has increased. The ratio of cereals to supplements in animal feed has changed through the years in an attempt to increase feeding efficiency and minimize feeding costs. Major factors contributing to feed efficiency are the genetic potential of the animal and the nutrients supplied to the animal. As feed efficiency has improved due to genetic enhancements, mineral and nutrient requirements for feed necessary to assure a complete and healthy diet have also risen. Since an animal's feed intake limits the amount of nutrients and calories it can consume, the feed industry has had to develop ways to make feeds that have improved protein quality, improved balance of essential amino acids, and increased metabolizable energy (oil).

Sources of feed protein, especially animal-derived protein, have come under global public scrutiny because of the bovine spongiform encephalopathy, or mad cow disease, crisis associated with the feeding of meat and bone meal as the primary protein source in animal diets in many parts of the world. Plant protein sources have become a dominant alternative protein supplement used in feed following bans on using meat and bone meal.

Plant protein sources, however, may lack sufficient levels of essential nutrients required for adequate animal health, growth and performance. Requirements vary depending on the species and age of the animal. For example, the order of the top three limiting amino acids in feed composed of corn and soybean meal is lysine, threonine, and tryptophan for swine, and methionine, lysine, and threonine for poultry. (FAO Animal Production and Health Proceedings, Protein Sources for the Animal Feed Industry, xi-xxv, 161-183 (2004)). These limiting amino acids must be available at specific minimum levels for the animals to use dietary protein efficiently. (Johnson et al. “Identification of Valuable Corn Quality Traits for Livestock Feed”, Report from the Center for Crops Utilization Research, Iowa State University, 1-22 (1999)). Furthermore, crude protein in feed ingredients is not totally digestible for any species. For example, corn protein is approximately 84% digestible by poultry and 82% digestible by swine (Johnson et al. (1999)). One method of increasing the nutritional quality of feed is to decrease crude protein in feed and supplement the feed with amino acids.

In addition to improving protein and amino acid composition, the feed industry has also had to develop ways to make feeds that are more calorie dense, such as by adding fat to the feed, often in the form of a liquid such as oil. Fat has the advantage of supplying calories to each mouthful of feed. However, adding fat to feed has disadvantages such as increased cost, added labor, and technical difficulties associated with automatic feeding systems. Additionally, the fat is often of poor quality, thus reducing the overall quality of the feed. To reduce the use of liquid fat in feed, the industry has tried increasing the oil content of the grain used in feed. This extra oil in the grain reduces and may eliminate the need for the addition of liquid fat to the feed.

Each of the various ingredients necessary to produce the right combination of nutrients (i.e. protein, amino acids, enzymes, etc.) will need to be transported from site of production and/or processing to the site of the end-user. The availability, price, and transportation requirements and costs of each component of a particular feed will vary from year to year and in different geographical regions. Because of the variability of the supply and cost of nutrients and additives, livestock feeders and feed manufacturers would value plants with traits that decrease the need for more expensive feedstuffs and additives and that can deliver increased nutrients in the same volume of grain.

Because feed is around 60% of animal production costs, any savings in feed costs can be considerable, especially in large operations. For example, nutritionally enhanced corn which can deliver higher levels of important nutrients and metabolizable energy, and/or enhanced digestibility and bioavailability of nutrients would provide the following benefits: reduced feed costs per unit weight gain or production of eggs or milk; reduced animal waste, particularly nitrogen and phosphorous; reduced veterinary costs and improved disease resistance; improved processing characteristics to make the feed; and improved quality (Johnson, et al. (1999)). Cost savings can be achieved by using nutritionally enhanced plants such as corn through, for example, reduced cost for needed supplements and synthetic additives, reduced transportation costs associated with the shipping of each additive and ingredients to produce the additives, reduced cost in mixing numerous additives during feed processing, and reduced costs associated with disposal of excess volume of manure. Much effort has been instituted academically and industry-wide to improve the nutritional composition of feed grain. Both traditional plant breeding and biotechnology techniques have been used to develop plants with desirable traits. For example, U.S. Pat. No. 5,723,730 describes an inbred corn line used to produce a hybrid with elevated percent oil and protein in grain. U.S. Pat. No. 6,268,550 suggests that an increase in acetyl CoA carboxylase (ACCase) activity during the early to mid stages of soybean plant development leads to an increase in oil content. Zeh (Plant Physiol., 2001, 127: 792-802) describes increasing the methionine content in potato plants by inhibiting threonine synthase using antisense technology. U.S. Pat. No. 5,589,616 discloses producing higher amounts of amino acids in plants by overexpressing a monocot storage protein. Similar approaches have been used in U.S. Pat. No. 4,886,878, U.S. Pat. No. 5,082,993 and U.S. Pat. No. 5,670,635. Other methods for increasing amino acids are disclosed in WO 95/15392, WO 96/38574, WO 89/11789, and WO 93/19190. In these cases, specific enzymes in the amino acid biosynthetic pathway such as the dihydrodipicolinic acid synthase are deregulated leading to an increase in the production of lysine.

Examples of grain-based feed that provide improved animal nutrition and can reduce environmental impact of animal production are described by Chang et al. in U.S. Pat. Nos. 7,087,261 and 6,774,288 and in U.S. Publ. No. 2005/0246791.

Methods for producing plants having desirable high value traits are complex and involve particular difficulties or conditions. For example, high value traits are often associated with reduced plant vigor, yield, or seed viability.

There remains a need to develop plants with increased content in one or more of protein, oil, and/or one or more amino acids to reduce feed costs to supply improved quality food for both animals and humans. Crop plants, such as corn plants, having these desirable traits may be used as starting material for further breeding to develop additional inbred lines and hybrids with these traits.

### SUMMARY OF THE INVENTION

The present invention provides novel expression cassettes and methods for increasing one or more of protein, oil, or one or more amino acids in a plant. Recombinant constructs, vectors, and plant cells, plants or parts thereof, comprising the expression cassettes of the invention as well as methods for their production are also provided.

In one aspect, the invention provides an expression cassette for increasing one or more of protein, oil, or one or more amino acids in a plant comprising:
(a) a promoter that is functional in a plant;
(b) a nucleic acid molecule; and
(c) the first intron of the rice Metallothionein 1 gene (Met1-1),
   wherein the nucleic acid molecule is operably linked to the promoter, and expression of the nucleic acid molecule in a plant, plant cell, or plant part confers an increase in one or more of protein, oil, or one or more amino acids in a plant, plant cell, or plant part relative to a corresponding wild-type plant, plant cell, or plant part; and wherein the nucleic acid molecule comprises:
(i) the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49.

In another embodiment, the invention provides an expression cassette comprising:
(a) an ScBV promoter or a functional fragment thereof;
(b) a nucleic acid molecule; and
(c) an intron,
   wherein the nucleic acid molecule is operably linked to the promoter, and expression of the nucleic acid molecule in a plant, plant cell, or plant part confers an increase in one or more of protein, oil, or one or more amino acids in a plant, plant cell, or plant part relative to a corresponding wild-type plant, plant cell, or plant part; and wherein the nucleic acid molecule comprises:
(i) the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 and SEQ ID NO: 49.

In another embodiment, the invention provides an expression cassette comprising:
(a) a promoter that is functional in a plant; and
(b) a nucleic acid molecule,
   wherein the nucleic acid molecule is operably linked to the promoter, and expression of the nucleic acid molecule in a plant, plant cell, or plant part confers an increase in protein and one or more amino acids in a plant, plant cell, or plant part relative to a corresponding wild-type plant, plant cell, or plant part; and
   wherein the nucleic acid molecule comprises:
(i) the nucleotide sequence of SEQ ID NO: 1, SEQ ID NO: 3, SEQ ID NO: 16, SEQ ED NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49.

In further embodiments, the promoter is a constitutive promoter, a seed-preferred promoter, or a seed-specific promoter. The constitutive promoter may comprise:
(a) the nucleic acid sequence of SEQ ID NO: 8 or SEQ ID NO: 9;
(b) a nucleic acid sequence having at least 95% sequence identity to the nucleic acid sequence of SEQ ID NO: 8 or SEQ ID NO: 9, wherein said nucleic acid sequence has constitutive expression activity; or
(c) a fragment of the nucleic acid sequence of SEQ ID NO: 8 or SEQ ID NO: 9, wherein the fragment has constitutive expression activity.

The seed-specific promoter may be an embryo-specific promoter comprising:
(a) the nucleic acid sequence of SEQ ID NO: 7;
(b) a nucleic acid sequence having at least 95% sequence identity to the nucleic acid sequence of SEQ ID NO: 7, wherein said nucleic acid sequence has embryo-specific expression activity; or
(c) a fragment of the nucleic acid sequence of SEQ ID NO: 7, wherein the fragment has embryo-specific expression activity.

In one embodiment, the expression cassette comprising:
(a) a promoter that is functional in a plant; and
(b) a nucleic acid molecule,
   may further comprise an intron. In a further embodiment, the intron is a monocot intron. In some embodiments, expression cassettes of the invention comprise an intron that comprises the nucleic acid sequence of SEQ ID NO: 10 or a nucleic acid sequence having at least 90% sequence identity to SEQ ID NO: 10.

In one embodiment, expression cassettes of the invention further comprise a nucleic acid sequence encoding a transit peptide that targets the polypeptide to a plastid. In further embodiments, the transit peptide is a plastid-targeting peptide from a ferredoxin gene. The nucleic acid sequence encoding a transit peptide may comprise:
(a) the nucleic acid sequence of SEQ ID NO: 5 or 73;
(b) a nucleic acid sequence encoding SEQ ID NO: 6;
(c) a nucleic acid sequence having at least 95% sequence identity to SEQ ID NO: 5 or 73; or
(d) a nucleic acid sequence encoding a polypeptide having at least 95% sequence identity to SEQ ID NO: 6.

Expression cassettes of the invention may further comprise a terminator. In further embodiments, the terminator is a NOS terminator or comprises the nucleic acid sequence of SEQ ID NO: 11.

In one embodiment, the expression cassette comprises a promoter that comprises the nucleic acid sequence of SEQ ID NO: 8, a nucleic acid molecule that comprises the nucleic acid sequence of SEQ ID NO: 3, and an intron that comprises the nucleic acid sequence of SEQ ID NO: 10, wherein the expression cassette further comprises the nucleic acid sequence of SEQ ID NO: 11.

In one embodiment, expression cassettes of the invention confer an increase in oil in a plant, plant cell, or plant part relative to a corresponding wild-type plant, plant cell, or plant part.

In a further embodiment, the invention provides a recombinant construct comprising any of the aforementioned expression cassettes. The invention further provides vectors comprising at least one of the aforementioned expression cassettes or recombinant constructs.

The invention also provides a microorganism comprising at least one of the aforementioned expression cassettes, a recombinant construct comprising at least one of the aforementioned expression cassettes, or a vector comprising at least one of the aforementioned expression cassettes or the aforementioned recombinant constructs.

In another aspect the invention provides a plant, plant cell, or plant part comprising at least one of the aforementioned expression cassettes, or a recombinant construct comprising at least one of the aforementioned expression cassettes, wherein the plant, plant cell, or plant part has an increase in one or more of protein, oil, or one or more amino acids relative to a corresponding wild-type plant, plant cell, or plant part. In one embodiment, the plant, plant cell, or plant part has an increase in protein and one or more amino acids relative to a corresponding wild-type plant, plant cell, or plant part. In another embodiment, the plant, plant cell, or plant part has an increase in protein, oil, and one or more amino acids relative to a corresponding wild-type plant, plant cell, or plant part. In a further embodiment, the plant part is a seed.

The invention also provides a food or feed composition comprising the aforementioned plant, plant cell, or plant part. In some embodiments, the food or feed composition is not supplemented with additional protein, oil, or one or more amino acids or has reduced supplementation with protein, oil, or one or more amino acids relative to a food or feed composition comprising a corresponding wild-type plant, plant cell, or plant part. The feed composition may formulated to meet the dietary requirements of swine, poultry, cattle, companion animals, or fish.

Further, the invention provides a method for producing a transgenic plant, plant cell, or plant part having an increase in one or more of protein, oil or one or more amino acids, comprising

(a) transforming a plant, plant cell, or plant part with at least one of the aforementioned expression cassettes, a recombinant construct comprising at least one of the expression cassettes, or a vector comprising the recombinant construct or at least one expression cassette; and

(b) optionally regenerating from the plant, plant cell, or plant part a transgenic plant,

wherein the transgenic plant, plant cell, or plant part has increased content of protein, oil, and/or one or more amino acids relative to a corresponding wild-type plant, plant cell, or plant part.

In still another aspect, the invention provides a method for increasing one or more of protein, oil, or one or more amino acids in a plant, plant cell, or plant part relative to a corresponding wild-type plant, plant cell, or plant part, comprising:
(a) obtaining the aforementioned plant, plant cell, or plant part; and
(b) selecting a plant, plant cell, or plant part with an increase in one or more of protein, oil, or one or more amino acids.
   In further embodiments, the plant is a monocotyledonous plant or the plant cell or plant part is from a monocotyledonous plant.

In any of the aforementioned methods, the content of one or more amino acids in the plant, plant cell, or plant part may be increased relative to a corresponding wild-type plant, plant cell, or plant part, and the one or more amino acids may be selected from the group consisting of arginine, cysteine, lysine, methionine, threonine, and valine. In further embodiments, the content of two or more amino acids, the content of protein, and/or the content of oil in the plant, plant cell, or plant part is increased relative to a corresponding wild-type plant, plant cell, or plant part. In yet further embodiments, the content of two, three, four, five, or six amino acids in the plant, plant cell, or plant part is increased relative to a corresponding wild-type plant, plant cell, or plant part.

In another aspect, the invention provides a method of producing a food or feed composition comprising
(a) providing a plant, plant cell, or plant part comprising at least one of the aforementioned expression cassettes or recombinant constructs; and
(b) producing a food or feed composition comprising the plant, plant cell or plant part.

In yet another aspect, the invention provides a method for producing a hybrid maize plant or seed comprising crossing a first inbred parent maize plant with a second inbred parent maize plant and harvesting a resultant hybrid maize seed, wherein said first inbred parent maize plant or said second inbred parent maize plant comprises at least one of the aforementioned expression cassettes or a recombinant construct comprising at least one expression cassette, or wherein said first inbred parent maize plant or said second inbred parent maize plant is derived from a plant that comprises at least one of the aforementioned expression cassettes or a recombinant construct comprising at least one of the expression cassettes. The invention also relates to a hybrid maize plant or seed produced by this method and a maize plant or part thereof produced by growing this seed.

The invention also concerns a method for developing a maize plant or seed in a maize plant breeding program using plant breeding techniques comprising employing a maize plant or part thereof as a source of plant breeding material, wherein the maize plant or part thereof comprises at least one of the aforementioned expression cassettes or a recombinant construct comprising at least one of the expression cassettes. The plant breeding techniques may be selected from the group consisting of recurrent selection, backcrossing, pedigree breeding, restriction length polymorphism enhanced selection, genetic marker enhanced selection, and transformation techniques. The invention also relates to a hybrid maize plant or seed produced by this method and a maize plant or part thereof produced by growing this seed.

In another aspect, the invention provides a method of plant breeding comprising:
(a) obtaining the aforementioned hybrid maize seed;
(b) crossing a plant grown from the hybrid maize seed with a different maize plant; and
(c) selecting a resultant progeny having an increase in one or more of protein, oil, or one or more amino acids.

In yet another aspect, the invention provides a method for producing grain with an increase in one or more of protein, oil, or one or more amino acids, comprising:
(a) obtaining a seed of a first plant and a seed of a second plant, the first plant comprising at least one of the aforementioned expression cassettes or a recombinant construct comprising at least one of the cassettes;
(b) growing the seed under conditions that result in cross pollination between the plant produced from the seed of the first plant and the plant produced from the seed of the second plant; and
(c) harvesting grain from the progeny.

The invention also relates to grain produced by this method, wherein the grain has an increase in one or more of protein, oil or one or more amino acids relative to a corresponding wild-type grain. In one embodiment, the one or more amino acids is selected from the group consisting of arginine, cysteine, lysine, methionine, threonine, and valine. In a further embodiment, the grain is corn.

In a still further aspect, the invention provides a method of producing a maize plant with an increase in one or more of protein, oil, or one or more amino acids, comprising:
(a) growing a progeny plant produced by crossing a maize plant comprising at least one of the aforementioned expression cassettes or a recombinant construct comprising at least one of the expression cassettes with a second maize plant;
(b) crossing the progeny plant with itself or a different plant to produce a seed of a progeny plant of a subsequent generation;
(c) growing a progeny plant of a subsequent generation from said seed and crossing the progeny plant of a subsequent generation with itself or a different plant; and
(d) repeating steps (b) and (c) for an additional 0-5 generations to produce a maize plant.

In one embodiment, the maize plant produced by the method is an inbred maize plant. In another embodiment, the method further comprises crossing the inbred maize plant with a second, distinct inbred maize plant to produce an F1 hybrid maize plant.

### DESCRIPTION OF THE FIGURES

FIG. 1A-G shows an amino acid sequence alignment of TPS homologs. Conserved sequence motifs are underlined.

### DESCRIPTION OF THE PREFERRED EMBODIMENTS

Throughout this application, various publications are referenced. The disclosures of all of these publications and those references cited within those publications are hereby incorporated by reference in their entireties into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used. The term “about” as used herein is to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20%, preferably 10% up or down (higher or lower). The word “comprise,” “comprising,” “include,” “including,” and “includes” as used herein and in the following claims is intended to specify the presence of one or more stated features, integers, components, or steps, but they do not preclude the presence or addition of one or more other features, integers, components, steps, or groups thereof.

In one aspect, the invention provides various novel expression cassettes. In another aspect, the invention provides methods for overexpressing a homolog of a trehalose-6-phosphate synthase in a plant, plant cell, or plant part which in turn confers an increase in one or more of protein, oil and/or one or more amino acids relative to a corresponding wild-type plant, wherein various expression cassettes of the invention can be used.

The term “wild-type” as used herein refers to a plant cell, seed, plant component, plant part, plant tissue, plant organ, or whole plant that has not been genetically modified with a polynucleotide in accordance with the invention.

The term “overexpressing” or “overexpression” as used herein means the level of expression of a nucleic acid molecule or a protein in a plant, plant cell, or plant part is higher or increased relative to its expression in a reference plant, plant cell, or plant part grown under substantially identical conditions.

### 1. Expression Cassettes of the Invention

1.1 Basic Components

The expression cassettes of the present invention generally comprise at least two components:
(a) a promoter that is functional in plants, and
(b) a nucleic acid molecule operably linked to said promoter,

wherein expression of the nucleic acid molecule in a plant, plant cell, or plant part confers an increase in one or more of protein, oil, or one or more amino acids in a plant, plant cell, or plant part relative to a corresponding wild-type plant, plant cell, or plant part.

As used herein, the terms “nucleic acid molecule”, “gene”, “nucleic acid” and “polynucleotide” are interchangeable and refer to naturally occurring or synthetic or artificial nucleic acid or polynucleotide. The terms “nucleic acid molecule”, “gene”, “nucleic acid” and “polynucleotide” comprise DNA or RNA or any nucleotide analogue and polymers or hybrids thereof in either linear or branched, single- or double-stranded, sense or antisense form. The terms also encompass RNA/DNA hybrids. Unless otherwise indicated, a particular nucleic acid sequence also implicitly encompasses conservatively modified variants thereof such as, but not limited to, degenerate codon substitutions and complementary sequences as well as the sequence explicitly indicated. A skilled worker will recognize that DNA sequence polymorphisms, which lead to changes in the encoded amino acid sequence, may exist within a population. These genetic polymorphisms in a gene may exist between individuals within a population owing to natural variation. These natural variants usually bring about a variance of 1 to 5% in the nucleotide sequence of a particular gene. Each and every one of these nucleotide variations and resulting amino acid polymorphisms in the encoded polypeptide which are the result of natural variation and do not modify the functional activity are to be encompassed by the invention.

The terms “polypeptide” or “protein” are used interchangeably herein.

“Expression cassette” as used herein refers to a DNA molecule which includes sequences capable of directing expression of a particular nucleic acid sequence (e.g., which codes for a protein of interest) in an appropriate host cell, including regulatory sequences such as a promoter operably linked to a nucleic acid sequence of interest, optionally associated with transcription termination signals and/or other regulatory elements. An expression cassette may also comprise sequences required for proper translation of the nucleic acid sequence of interest. The expression cassette comprising the nucleic acid sequence of interest may be chimeric, meaning that at least one of its components is heterologous with respect to at least one of its other components. An expression cassette may be assembled entirely extracellularly (e.g., by recombinant cloning techniques).

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family. The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

The term “operably linked” or “operable linkage” encompasses, for example, an arrangement of the transcription regulating nucleotide sequence with the nucleic acid sequence to be expressed and, if appropriate, further regulatory elements, such as terminator or enhancers, in such a way that each of the regulatory elements can fulfill its intended function to allow, modify, facilitate or otherwise influence expression of the nucleic acid sequence under the appropriate conditions. Appropriate conditions relate to preferably the presence of the expression cassette in a plant cell. In a preferred arrangement, the nucleic acid sequence is placed down-stream (i.e. in 5′ to 3′-direction) of the transcription regulating nucleotide sequence. Optionally, additional sequences, such as a linker, multiple cloning site, intron, or nucleotide sequence encoding a protein targeting sequence may be inserted between the two sequences.

The term “heterologous” refers to material (nucleic acid or protein) which is obtained or derived from different source organisms, or, from different genes or proteins in the same source organism or a nucleic acid sequence to which it is not linked in nature or to which it is linked at a different location in nature. For example, a protein-coding nucleic acid sequence operably linked to a promoter which is not the native promoter of this protein-coding sequence, is considered to be heterologous to the promoter.

All percentages of protein, oil, and amino acid content in a plant, plant cell, or plant part recited herein are percent dry weight. Methods for determining and calculating the protein, oil, and amino acid content in a plant, plant cell, or plant part are known in the art and routinely used by a skilled person.

In one embodiment, the content of one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of the corresponding one or more amino acids in a corresponding wild-type plant, plant cell, or plant part. Preferably, the amino acids, of which the content is increased in the plant, plant cell, or plant part of the invention, are selected from the group consisting of arginine, cysteine, lysine, methionine, threonine, and valine. More preferably, the plant, plant cell, or plant part of the invention demonstrates an increased content in one or more amino acids selected from the group consisting of arginine, cysteine, lysine, methionine, threonine, and valine by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% relative to a corresponding wild-type plant, plant cell, or plant part. In other embodiments, the increased content of one or more amino acids is an increase in two, three, four, five, or six amino acids selected from the group consisting of arginine, cysteine, lysine, methionine, threonine, and valine.

In another embodiment, the oil content of the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the oil content of the corresponding wild-type plant, plant cell, or plant part.

In yet another embodiment, the protein content of the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the protein content of the corresponding wild-type plant, plant cell, or plant part.

In a further embodiment, the content of protein and one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of protein and one or more amino acids in a corresponding wild-type plant, plant cell, or plant part.

In yet a further embodiment, the content of protein, oil, and one or more amino acids in the plant, plant cell, or plant part of the invention is increased by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, or 200% over the content of protein, oil, and one or more amino acids in a corresponding wild-type plant, plant cell, or plant part.

1.1.1 Promoters

The term “promoter” as used herein is equivalent of the terms “promoter element,” “promoter sequence,” or “transcription regulating nucleotide sequence” and refers to a DNA sequence which, when linked to a nucleic acid sequence of interest, is capable of controlling the transcription of the nucleic acid sequence of interest into mRNA. A transcription regulating nucleotide sequence or a promoter is typically, though not necessarily, located 5′ (i.e. upstream) of a nucleic acid sequence of interest (e.g., proximal to the transcriptional start site of a structural gene) whose transcription into mRNA it controls, and provides a site for specific binding by RNA polymerase and other transcription factors for initiation of transcription.

For expressing a nucleic acid molecule of interest according to the present invention, the nucleic acid molecule of interest is be operably linked to an appropriate promoter, preferably a promoter that is functional in a plant. The term “promoter that is functional in a plant” means principally any promoter which is capable of driving the expression of a nucleic acid operably linked thereto, in particular foreign nucleic acid sequences or genes, in plants or plant parts, plant cells, plant tissues, plant cultures. Unless otherwise specified in a particular embodiment, the expression specificity of said promoter that is functional in plants can be for example constitutive, inducible, developmentally regulated, tissue-specific or tissue-preferential, organ-specific or organ-preferential, cell type-specific or cell type-preferential, spatial-specific or spatial-preferential, and/or temporal-specific or temporal-preferential.

Such promoters include, but not limited to, those that can be obtained from plants, plant viruses and bacteria that contain genes that are expressed in plants, such as Agrobacterium and Rhizobium.

Constitutive promoters are generally active under most environmental conditions and states of development or cell differentiation. Useful constitutive promoters for plants include those obtained from Ti- or Ri-plasmids, from plant cells, plant viruses or other organisms whose promoters are found to be functional in plants. Bacterial promoters that function in plants, and thus are suitable for use in the present invention include, but not limited to, the octopine synthetase promoter, the nopaline synthase promoter, and the mannopine synthetase promoter from the T-DNA of Agrobacterium. Likewise, viral promoters that function in plants can also be used in the present invention. Examples of viral promoters include, but are not limited to, the promoter isolated from sugarcane bacilliform virus (ScBV; U.S. Pat. No. 6,489,462; Nadiya et al., Biotechnology, 2010, published online), the cauliflower mosaic virus (CaMV) 35S transcription initiation region (Franck et al., Cell, 1980, 21: 285-294; Odell et al., Nature, 1985, 313: 810-812; Shewmaker et al., Virology, 1985, 140: 281-288; Gardner et al., Plant Mol. Biol., 1986, 6: 221-228), the cauliflower mosaic virus (CaMV) 19S transcription initiation region (U.S. Pat. No. 5,352,605 and WO 84/02913) and region VI promoters, and the full-length transcript promoter from Figwort mosaic virus. Other suitable constitutive promoters for use in plants include, but are not limited to, actin promoters such as the rice actin promoter (McElroy et al., Plant Cell, 1990, 2: 163-171) or the Arabidopsis actin promoter, histone promoters, tubulin promoters, or the mannopine synthase promoter (MAS), ubiquitin or poly-ubiquitin promoters (Sun and Callis, Plant J., 1997, 11(5): 1017-1027; Cristensen et al., Plant Mol. Biol., 1992, 18: 675-689; Christensen et al., Plant Mol. Biol., 1989 12: 619-632; Bruce et al., Proc. Natl. Acad. Sci. USA, 1989, 86: 9692-9696; Holtorf et al., Plant Mol. Biol., 1995, 29: 637-649; for example, the ubiquitin promoter from Zea mays (SEQ ID NO: 70)), the Mac or DoubleMac promoters (U.S. Pat. No. 5,106,739; Comai et al., Plant Mol. Biol., 1990, 15: 373-381), Rubisco small subunit (SSU) promoter (U.S. Pat. No. 4,962,028), the legumin B promoter (GenBank Acc. No. X03677), the TR dual promoter, the Smas promoter (Yellen et al., EMBO J., 1984, 3: 2723-2730), the cinnamyl alcohol dehydrogenase promoter (U.S. Pat. No. 5,683,439), the promoters of the vacuolar ATPase subunits, the pEMU promoter (Last et al., Theor. Appl. Genet., 1991, 81: 581-588), the maize H3 histone promoter (Lepetit et al., Mol. Gen. Genet., 1992, 231: 276-285; Atanassova et al., Plant J., 1992, 2(3): 291-300), β-conglycinin promoter, the phaseolin promoter, the ADH promoter, and heat-shock promoters, the nitrilase promoter from Arabidopsis thaliana (WO 03/008596; GenBank Acc. No. U38846, nucleotides 3,862 to 5,325 or else 5,342), promoter of a proline-rich protein from wheat (WO 91/13991), the promoter of the Pisum sativum ptxA gene, and other promoters active in plant cells that are known to those of skill in the art.

In some embodiments, the expression cassettes of the invention comprise a constitutive promoter. Preferably, the constitutive promoter is isolated from sugarcane bacilliform virus (ScBV). More preferably, the constitutive promoter to be included in the expression cassettes of the invention comprises:

(a) the nucleotide sequence of SEQ ID NO: 8 or 9;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 8 or 9, wherein said nucleotide sequence has constitutive expression activity; or
(c) a fragment of the nucleotide sequence of SEQ ID NO: 8 or 9, wherein the fragment has constitutive expression activity.

Inducible promoters are active under certain environmental conditions, such as the presence or absence of a nutrient or metabolite, heat or cold, light, pathogen attack, anaerobic conditions, and the like. Examples for such promoters are provided in WO 95/19443, EP 388186, Gatz et al., Mol. Gen. Genetics, 1991, 227: 229-237, EP 335528, WO 93/21334, WO 93/01294, Schena et al., Proc. Natl. Acad. Sci. USA, 1991, 88: 10421, Ward et al., Plant Mol. Biol., 1993, 22: 361-366, U.S. Pat. No. 5,187,267, WO 96/12814, and EP 0375091.

A cell-specific or cell-preferential, tissue-specific or tissue-preferential, or organ-specific or organ-preferential promoter is one that is capable of preferentially initiating transcription in certain types of cells, tissues, or organs, such as leaves, stems, roots, flowers, fruits, anthers, ovaries, pollen, seed tissue, green tissue, or meristem. A promoter is cell-, tissue- or organ-specific or preferential, if its activity, measured on the amount of RNA produced under control of the promoter, is at least 30%, 40%, 50%, preferably at least 60%, 70%, 80%, 90%, more preferably at least 100%, 200%, 300%, higher in a particular cell-type, tissue or organ, then in other cell-types or tissues of the same plant, preferably the other cell-types or tissues are cell types or tissues of the same plant organ, e.g., leaves or roots. In the case of organ specific or preferential promoters, the promoter activity has to be compared to the promoter activity in other plant organs, e.g., leaves, stems, flowers or seeds. For example, the tissue-specific ES promoter from tomato is particularly useful for directing expression in fruits (see, e.g., Lincoln et al., Proc. Natl. Acad. Sci. USA, 1988, 84: 2793-2797; Deikman et al., EMBO J., 1988, 7: 3315-3320; Deikman et al., Plant Physiol., 1992, 100: 2013-2017). Seed-specific or seed-preferential promoters are preferentially expressed during seed development and/or germination, which can be embryo-, endosperm-, and/or seed coat-specific or preferential. See Thompson et al., BioEs-says, 1989, 10: 108. Examples of seed-specific or preferential promoters include, but are not limited to, those derived from the globulin 1 gene from maize (ZmGlb1) (for example, SEQ ID NO: 7) (Belanger et al., Genetics, 1991, 129: 863-872), the zein genes from maize, including 10 kDa zein (for example, SEQ ID NO: 71), 19 kDa zein, and 27 kDa zein (for example, SEQ ID NO: 15), the MAC1 gene from maize (Sheridan et al., Genetics, 1996, 142: 1009-1020), the Cat3 gene from maize (GenBank Accession No. L05934), the gene encoding oleosin 18 kD from maize (GenBank Accession No. J05212), viviparous-1 gene from Arabidopsis (Genbank Accession No. U93215), the gene encoding oleosin from Arabidopsis (Genbank Accession No. Z17657), the Atmyc1 gene from Arabidopsis (Urao et al., Plant Mol. Biol., 1996, 32: 571-576), the 2S seed storage protein gene family from Arabidopsis (Conceicao et al., Plant J., 1994, 5: 493-505), the gene encoding oleosin 20 kD from Brassica napus (GenBank Accession No. M63985), the napin gene from Brassica napus (GenBank Accession No. J02798; Joseffson et al., J. Biol. Chem., 1987, 262: 12196-12201), the napin gene family (e.g., from Brassica napus; Sjodahl et al., Planta, 1995, 197: 264-271, U.S. Pat. No. 5,608,152; Stalberg et al., Planta, 1996, 199: 515-519), the gene encoding the 2S storage protein from Brassica napus (Dasgupta et al., Gene, 1993, 133: 301-302), the genes encoding oleosin A (Genbank Accession No. U09118) and oleosin B (Genbank Accession No. U09119) from soybean, the gene encoding low molecular weight sulphur rich protein from soybean (Choi et al., Mol. Gen. Genet., 1995, 246: 266-268), the phaseolin gene (U.S. Pat. No. 5,504,200; Bustos et al., Plant Cell, 1989, 1(9): 839-853; Murai et al., Science, 1983, 23: 476-482; Sengupta-Gopalan et al., Proc. Natl. Acad. Sci. USA, 1985, 82: 3320-3324), the 2S albumin gene, the legumin gene (Shirsat et al., Mol. Gen. Genet., 1989, 215(2): 326-331), the USP (unknown seed protein) gene, the sucrose binding protein gene (WO 00/26388), the legumin B4 gene (LeB4; Fiedler et al., Biotechnology, 1995, 13(10): 1090-1093; Baumlein et al., Plant J., 1992, 2(2): 233-239; Baumlein et al., Mol. Gen. Genet., 1991, 225(3): 459-467; Baumlein et al., Mol. Gen. Genet., 1991, 225: 121-128), the Arabidopsis oleosin gene (WO 98/45461), the Brassica Bce4 gene (WO 91/13980), genes encoding the “high-molecular-weight glutenin” (BMWG), gliadin, branching enzyme, ADP-glucose pyrophosphatase (AGPase) or starch synthase. Further seed specific or preferential promoters include the KG86_{—}12a promoter (SEQ ID NO: 14) and the KG86 promoter (SEQ ID NO: 77).

Other suitable tissue- or organ-specific or preferential promoters include a leaf-specific and light-induced promoter such as that from cab or Rubisco (Timko et al., Nature, 1985, 318: 579-582; Simpson et al., EMBO J., 1985, 4: 2723-2729), an anther-specific promoter such as that from LAT52 (Twell et al., Mol. Gen. Genet., 1989, 217: 240-245), a pollen-specific promoter such as that from Zml3 (Guerrero et al., Mol. Gen. Genet., 1993, 224: 161-168), and a microspore-preferred promoter such as that from apg (Twell et al., Sex. Plant Reprod., 1983, 6: 217-224). Also suitable promoters are, for example, specific promoters for tubers, storage roots or roots such as, for example, the class I patatin promoter (B33), the potato cathepsin D inhibitor promoter, the starch synthase (GBSS1) promoter or the sporamin promoter, and fruit-specific promoters such as, for example, the tomato fruit-specific promoter (EP 0409625). Promoters which are furthermore suitable are those which ensure leaf-specific or leaf-preferential expression. Further examples of promoters which may be mentioned are the potato cytosolic FBPase promoter (WO 98/18940), the Rubisco (ribulose-1,5-bisphosphate carboxylase) SSU (small subunit) promoter or the potato ST-LSI promoter (Stockhaus et al., EMBO J., 1989, 8(9): 2445-2451). Other suitable promoters are those which govern expression in seeds and plant embryos. Further suitable promoters are, for example, fruit-maturation-specific promoters such as, for example, the tomato fruit-maturation-specific promoter (WO 94/21794), flower-specific promoters such as, for example, the phytoene synthase promoter (WO 92/16635) or the promoter of the P1-rr gene (WO 98/22593) or another node-specific promoter as described in EP 0249676 may be used advantageously. The promoter may also be a pith-specific promoter, such as the promoter isolated from a plant TrpA gene as described in WO 93/07278.

In some embodiments, the expression cassettes of the invention comprise a tissue-specific or tissue-preferential promoter. More preferably, the tissue-specific or tissue-preferential promoter is a seed-specific or seed-preferential promoter, an endosperm-specific or endosperm-preferential promoter, or an embryo-specific or embryo-preferential promoter.

In some preferred embodiments, the promoter to be included in the expression cassettes of the invention is an embryo-specific or embryo-preferential promoter, preferably an embryo-specific or embryo-preferential promoter comprising:

(a) the nucleotide sequence of SEQ ID NO: 7;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 7, wherein said nucleotide sequence has embryo-specific or embryo-preferential expression activity; or

(c) a fragment of the nucleotide sequence of SEQ ID NO: 7, wherein the fragment has embryo-specific or embryo-preferential expression activity.

In another embodiment, the seed-specific or seed-preferential promoter may comprise
(a) the nucleic acid sequence of SEQ ID NO: 14 or 15 or 71 or 77;
(b) a nucleic acid sequence having at least 95%, preferably 96%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 14 or 15 or 71 or 77, wherein said nucleic acid sequence has endosperm-specific or endosperm-preferential expression activity or whole-seed preferential or whole-seed specific expression activity; or
(c) a fragment of the nucleic acid sequence of SEQ ID NO: 14 or 15 or 71 or 77, wherein the fragment has endosperm-specific or endosperm-preferential expression activity or whole-seed preferential or whole-seed specific expression activity.

Developmentally regulated or developmental stage-preferential promoters are preferentially expressed at certain stages of development. Suitable developmental regulated promoters include, but not limited to, fruit-maturation-specific promoters, such as, for example, the fruit-maturation-specific promoter from tomato (WO 94/21794, EP 0409625). Developmental regulated promoters also include partly the tissue-specific or tissue-preferential promoters described above since individual tissues are, naturally, formed as a function of the development. An example of a development-regulated promoter is described in Baerson et al. (Plant Mol. Biol., 1993, 22(2): 255-267).

Other promoters or promoter elements suitable for the expression cassettes of the invention include, but not limited to, promoters or promoter elements capable of modifying the expression-governing characteristics. Thus, for example, the tissue-specific or tissue-preferential expression may take place in addition as a function of certain stress factors, owing to genetic control sequences. Such elements are, for example, described for water stress, abscisic acid (Lam and Chua, J. Biol. Chem., 1991, 266(26): 17131-17135) and heat stress (Schoffl et al., Molecular & General Genetics, 1989, 217(2-3): 246-253).

Unless specifically provided herein, the promoter to be included in the expression cassettes of the invention is a promoter that is functional in a plant.

1.1.2 Trehalose-6-Phosphate Synthase (TPS) Homologs

Trehalose is the most widespread disaccharide in nature, occurring in bacteria, fungi, insects, and plants. In most cases, trehalose synthesis is a two-step process. In the first step, trehalose-6-phosphate (T6P) is synthesized from uridine diphosphate glucose (UDP-G) and glucose-6-phosphate (G6P) by trehalose-6-phosphate synthase (TPS, EC 2.4.1.15). In the second step, trehalose-6-phosphate is dephosphorylated to trehalose by T6P phosphatase (TPP).

In Arabidopsis, 21 putative trehalose biosynthesis genes are classified in three subfamilies (Class I, II and III) based on their similarity with yeast TPS and TPP genes. The Class I proteins (AtTPS1-AtTPS4) contain a TPS domain, Class II proteins (AtTPS5-AtTPS11) contain both a TPS domain and a TPP domain, and the Class III subfamily proteins are characterized by having only a TPP domain. Although the Arabidopsis Class I and Class III proteins have established TPS and TPP activity, respectively, the function of the Class II proteins (AtTPS5-AtTPS11) remains elusive. Heterologous expression of class II type proteins in yeast indicated that none of the encoded enzymes displayed significant TPS or TPP activity (Ramon, M. et al., Plant Cell Environ 32:1015-1032, 2009). For example, the class II AtTPS6 was shown to regulate plant architecture, shape of epidermal pavement cells, and branching of trichomes (Chary, S, N., et al., Plant Physiol 146: 97-107, 2008), indicating a role of the gene in controlling cellular morphogenesis. Many TPS homologs contain two conserved Pfam domains, the Pfam:PF00982.15 glycosyltransferase family 20 domain and the Pfam:PF02358.10 trehalose-phosphatase domain.

It is found that, by expressing certain TPS homologs in a plant, plant cell, or plant part under control of some specific types of promoters, optionally in combination with other regulatory elements and/or targeting peptides, the content of one or more of protein, oil, or one or more amino acids in such a plant, plant cell, or plant part is surprisingly increased. Accordingly, in one aspect, the invention provides an expression cassette capable of expressing a nucleic acid molecule encoding a TPS homolog in a plant, plant cell, or plant part, wherein the expression of such a nucleic acid molecule confers increased content in one or more of protein, oil, or one or more amino acids in said plant, plant cell, or plant part relative to a corresponding wild-type plant, plant cell, or plant part. Preferably, the expression of the nucleic acid molecule encoding a TPS homolog confers an increase in protein and one or more amino acids in a plant, plant cell, or plant part relative to a corresponding wild-type plant, plant cell, or plant part. In another embodiment, the expression of the nucleic acid molecule encoding a TPS homolog confers an increase in oil and one or more amino acids in a plant, plant cell, or plant part relative to a corresponding wild-type plant, plant cell, or plant part. More preferably, the expression of the nucleic acid molecule encoding a TPS homolog confers an increase in protein, oil, and one or more amino acids in a plant, plant cell, or plant part relative to a corresponding wild-type plant, plant cell, or plant part.

Preferably, the TPS homolog suitable for the present invention comprises a Pfam:PF00982.15 glycosyltransferase family 20 domain and a Pfam:PF02358.10 trehalose-phosphatase domain. Accordingly, in one embodiment, the nucleic acid molecule encoding a TPS homolog to be included in the expression cassettes of the invention comprises a polynucleotide sequence encoding a polypeptide having the amino acid sequence of SEQ ID NO: 2, 4, 17, 19, 21, 23, 25, 27, 29, 31, 33, 35 or 37, or functional variants thereof. In another embodiment, the nucleic acid molecule encoding a TPS homolog comprises the polynucleotide sequence of SEQ ID NO: 1, 3, 16, 18, 20, 22, 24, 26, 28, 30, 32, 34, 36, 50 or 51 or functional variants thereof.

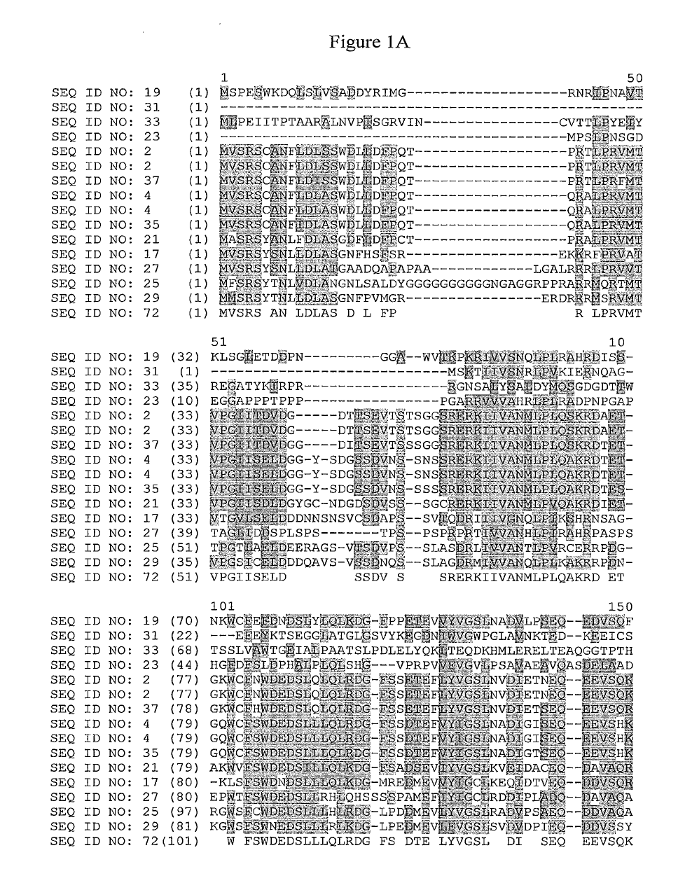

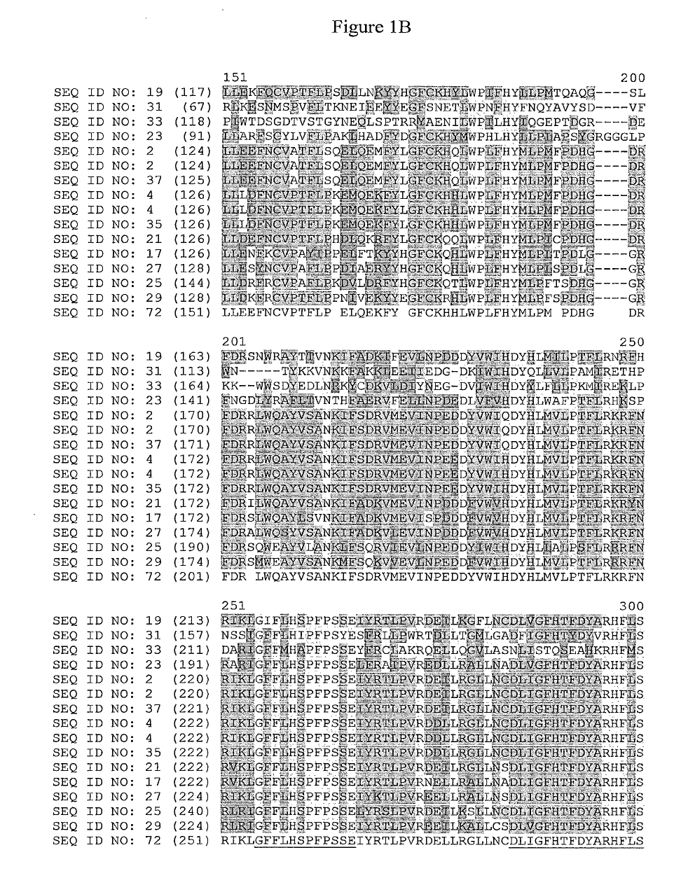

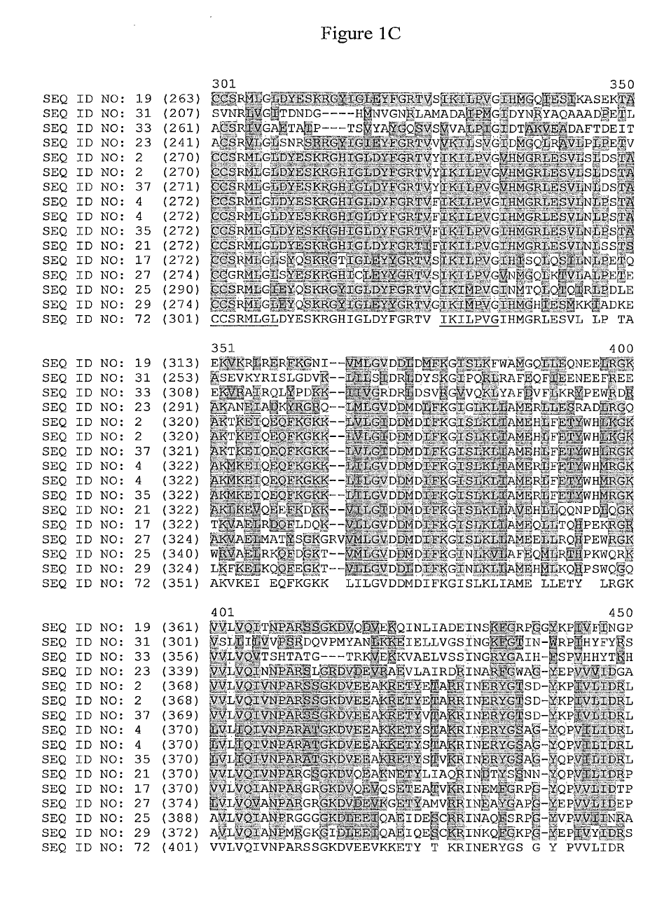

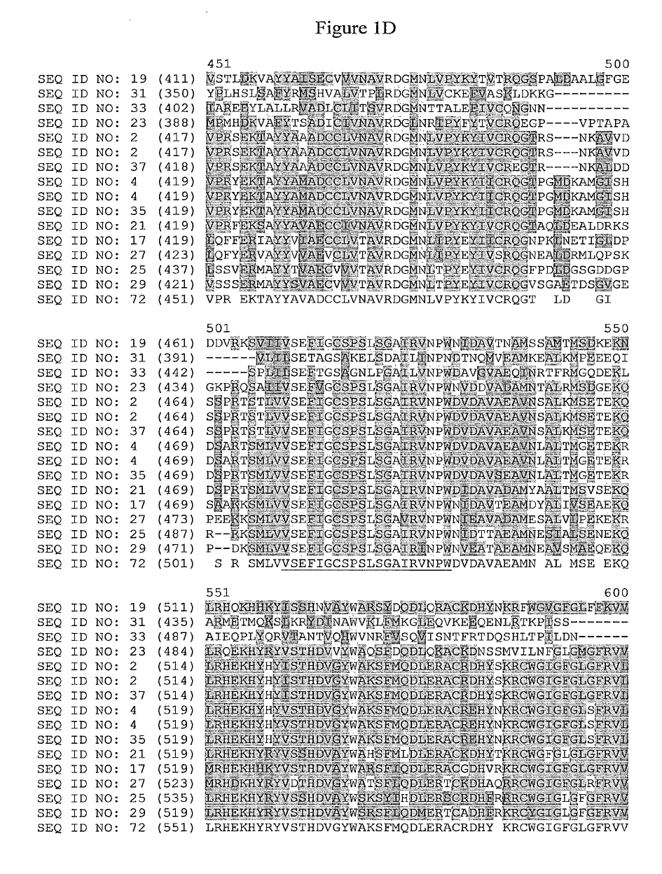

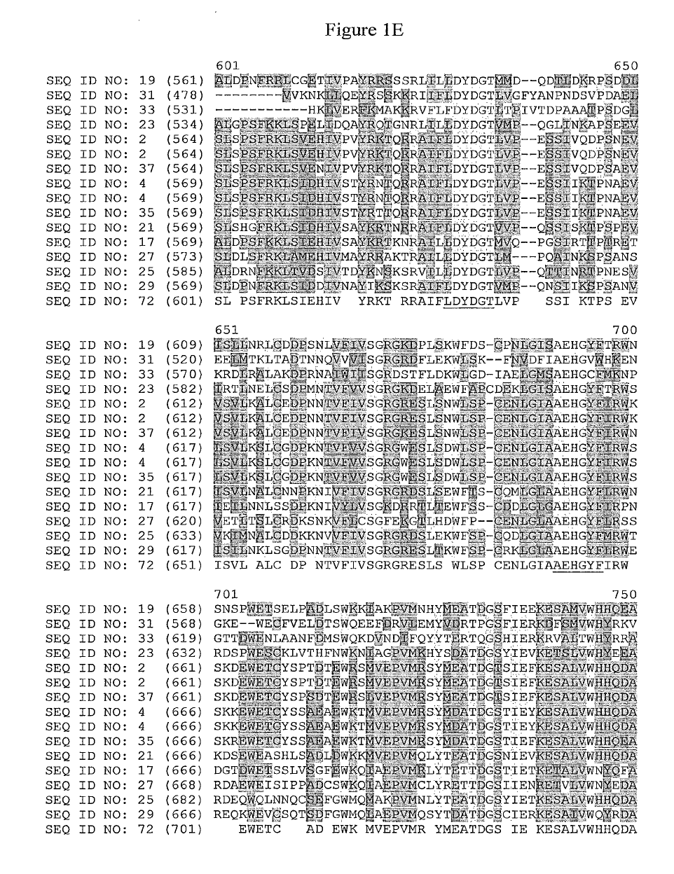

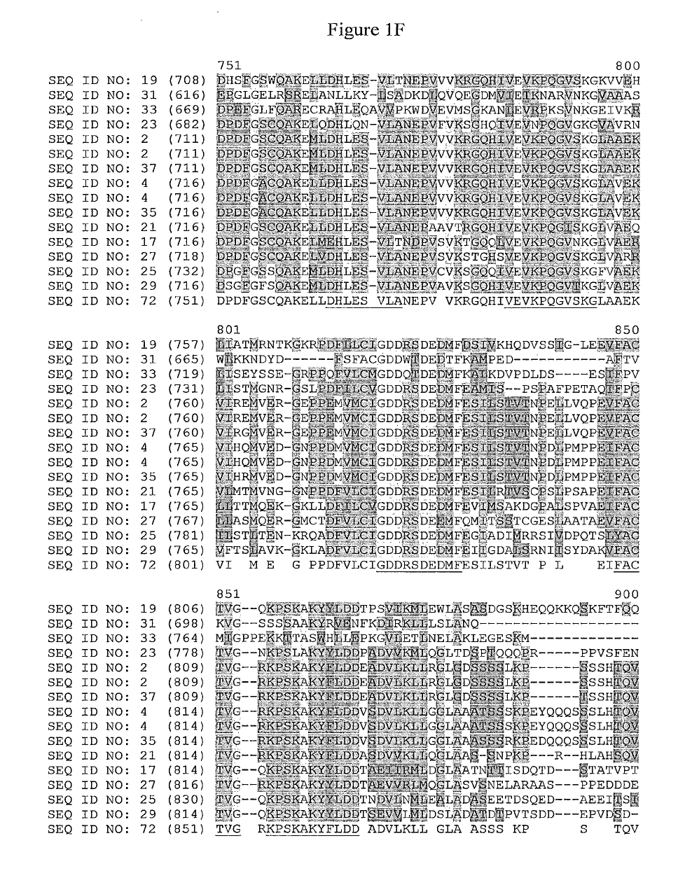

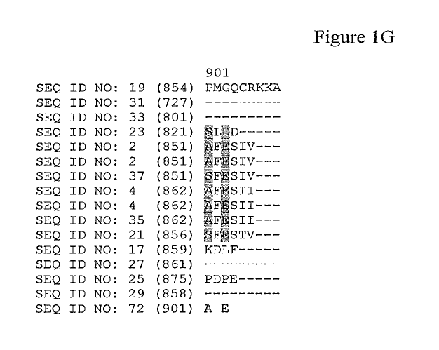

The TPS homolog may also contain specific amino acid sequence motifs within each Pfam domain. For example, the PF00982.15 Pfam domain contains the amino acid sequence motifs of SEQ ID NO: 38, 39, 40, 41 and 42 and the PF02358.10 Pfam domain contains the amino acid sequence motifs of SEQ ID NO: 43, 44, 45, 46, 47, 48 and 49, as shown in FIG. 1. In a preferred embodiment, the nucleic acid molecule encoding a TPS homolog comprises a nucleotide sequence encoding a polypeptide having the amino acid sequence motifs of SEQ ID NO: 38, 39, 40, 41, 42, 43, 44, 45, 46, 47, 48 and 49.

AtTPS8 and AtTPS9 are Arabidopsis Class II trehalose-6-phosphate synthases that contain the PF00982.15 and PF02358.10 Pfam domains. AtTPS8 and AtTPS9 also contain the amino acid sequence motifs of SEQ ID NO: 38, 39, 40, 41, 42, 43, 44, 45, 46, 47, 48 and 49. In a preferred embodiment, the expression cassette of the invention contains a nucleic acid molecule encoding AtTPS8 or AtTPS9.

The percent sequence identity of several TPS homologs to AtTPS8 or AtTPS9 is shown in Table 1 below. Table 2 shows the location of the PF00982.15 and PF02358.10 Pfam domains, as well as the percent sequence identity between the Pfam domains of the TPS homologs and the Pfam domains of AtTPS8 or AtTPS9. All of the TPS homologs shown in Tables 1 and 2 contain the conserved amino acid sequence motifs of SEQ ID NO: 38, 39, 40, 41, 42, 43, 44, 45, 46, 47, 48 and 49, as shown in FIG. 1.

TABLE 1
Percent sequence identity of TPS homologs to AtTPS8
(SEQ ID NO: 2) or AtTPS9 (SEQ ID NO: 4).

| | | % Identity to | % Identity to |
|---|---|---|---|
| SEQ | | AtTPS8 | AtTPS9 |
| ID NO | ORGANISM | (SEQ ID NO: 2) | (SEQ ID NO: 4) |
| 2 | Arabidopsis thaliana | 100 | 83.3 |
| 4 | Arabidopsis thaliana | 83.3 | 100 |
| 17 | Arabidopsis thaliana | 59.4 | 61.2 |
| 19 | Arabidopsis thaliana | 57.3 | 57.6 |
| 21 | Glycine max | 73.3 | 74.4 |
| 23 | Oryza sativa | 49.8 | 49.5 |
| 25 | Oryza sativa | 56.4 | 56.3 |
| 27 | Oryza sativa | 59.1 | 59.3 |
| 29 | Solanum tuberosum | 59.7 | 59.4 |
| 31 | Crocosphaera watsonii | 29.6 | 29.6 |
| 33 | Yarrowia lipolytica | 28.9 | 29.2 |
| 35 | Arabidopsis lyrata | 83.3 | 98.2 |
| | subsp. lyrata | | |
| 37 | Arabidopsis lyrata | 97.0 | 83.3 |
| | subsp. lyrata | | |
| 54 | Arabidopsis thaliana | 28.7 | 28.5 |
| 56 | Sorghum bicolor | 27.9 | 28.9 |
| 58 | Solanum lycopersicum | 29.1 | 29.1 |
| 60 | Triticum aestivum | 28.1 | 28.0 |
| 62 | Zostera marina | 56.0 | 56.2 |
| 64 | Zea mays | 63.5 | 63.3 |
| 66 | Zea mays | 54.8 | 54.7 |
| 68 | Zea mays | 59.2 | 58.8 |

TABLE 2
Pfam domains PF00982.15 and PF02358.10 in the amino acid sequences of TPS homologs.
% Identity = the percent sequence identity of the Pfam domain of the TPS
homolog to the Pfam domain of AtTPS8 (SEQ ID NO: 2) or AtTPS9 (SEQ ID NO: 4).

| | | PFAM Domain PF00982.15 | | | | PFAM Domain PF02358.10 | | | |
|---|---|---|---|---|---|---|---|---|---|
| | | Glycosyltransferase family 20 | | | | Trehalose-phosphatase | | | |
| SEQ | | | | % Identity | % Identity | | | % Identity | % Identity |
| ID | | Pfam | Pfam | to AtTPS8 | to AtTPS9 | Pfam | Pfam | to AtTPS8 | to AtTPS9 |
| NO | ORGANISM | Start | End | PFAM | PFAM | Start | End | PFAM | PFAM |
| 2 | Arabidopsis | 57 | 541 | 100.0 | 86.1 | 590 | 825 | 100.0 | 83.5 |
| | thaliana | | | | | | | | |
| 4 | Arabidopsis | 59 | 546 | 86.1 | 100.0 | 595 | 830 | 83.5 | 100.0 |
| | thaliana | | | | | | | | |
| 17 | Arabidopsis | 60 | 546 | 65.0 | 67.0 | 595 | 830 | 56.8 | 58.1 |
| | thaliana | | | | | | | | |
| 19 | Arabidopsis | 50 | 538 | 63.4 | 62.8 | 587 | 822 | 60.8 | 60.8 |
| | thaliana | | | | | | | | |
| 21 | Glycine max | 59 | 546 | 77.3 | 77.3 | 595 | 830 | 71.6 | 73.7 |
| 23 | Oryza sativa | 23 | 511 | 52.5 | 53.3 | 560 | 794 | 56.1 | 58.6 |
| 25 | Oryza sativa | 77 | 562 | 60.7 | 60.9 | 611 | 846 | 61.4 | 60.2 |
| 27 | Oryza sativa | 59 | 550 | 64.0 | 65.7 | 599 | 832 | 58.5 | 58.5 |
| 29 | Solanum | 61 | 546 | 63.3 | 63.1 | 595 | 830 | 62.7 | 62.7 |
| | tuberosum | | | | | | | | |
| 31 | Crocosphaera | 2 | 462 | 32.9 | 33.2 | 496 | 714 | 33.2 | 34.0 |
| | watsonii | | | | | | | | |
| 33 | Yarrowia | 22 | 514 | 27.5 | 27.7 | 546 | 782 | 36.0 | 36.1 |
| | lipolytica | | | | | | | | |
| 35 | Arabidopsis | 59 | 546 | 86.1 | 98.8 | 595 | 830 | 83.5 | 98.3 |
| | lyrata subsp. | | | | | | | | |
| | lyrata | | | | | | | | |
| 37 | Arabidopsis | 58 | 541 | 97.5 | 86.9 | 590 | 825 | 97.5 | 83.5 |
| | lyrata subsp. | | | | | | | | |
| | lyrata | | | | | | | | |
| 54 | Arabidopsis | 4 | 471 | 37.4 | 37.6 | 515 | 755 | 29.6 | 29.3 |
| | thaliana | | | | | | | | |
| 56 | Sorghum | 128 | 595 | 37.6 | 37.6 | 643 | 872 | 29.3 | 28.1 |
| | bicolor | | | | | | | | |
| 58 | Solanum | 91 | 558 | 37.6 | 37.0 | 602 | 843 | 30.2 | 29.5 |
| | lycopersicum | | | | | | | | |
| 60 | Triticum | 3 | 470 | 37.2 | 37.0 | 518 | 754 | 30.0 | 28.5 |
| | aestivum | | | | | | | | |
| 62 | Zostera | 67 | 552 | 59.9 | 59.8 | 601 | 833 | 60.2 | 58.9 |
| | marina | | | | | | | | |
| 64 | Zea mays | 58 | 544 | 66.2 | 66.0 | 593 | 829 | 65.0 | 67.1 |
| 66 | Zea mays | 80 | 582 | 60.5 | 59.7 | 631 | 865 | 60.3 | 59.9 |
| 68 | Zea mays | 59 | 546 | 62.7 | 62.3 | 595 | 830 | 63.0 | 63.6 |

As provided in Table 2, some TPS homologs comprise both a Pfam:PF00982.15 glycosyltransferase family 20 domain and a Pfam:PF02358.10 trehalose-phosphatase domain having significant sequence identity to those domains found in the TPS homologs as shown in SEQ ID NO: 2 or 4. Accordingly, in other embodiments, the TPS homologs suitable for the present invention may comprise a Pfam:PF00982.15 glycosyltransferase family 20 domain and a Pfam:PF02358.10 trehalose-phosphatase domain. In another embodiment, the TPS homologs suitable for the present invention may comprise a Pfam:PF00982.15 glycosyltransferase family 20 domain and a Pfam:PF02358.10 trehalose-phosphatase domain, wherein the Pfam:PF00982.15 glycosyltransferase family 20 domain has at least 50%, preferably 55%, 60%, 65%, 70%, 75%, 80%, 85%, 90%, 91%, 92%, 93%, 94%, 95%, 96%, 97%, 98%, or 99% identity to the amino acid residues 57 to 541 of SEQ ID NO: 2 or the amino acid residues 59 to 546 of SEQ ID NO: 4, and wherein the Pfam:PF02358.10 trehalose-phosphatase domain has at least 55%, preferably 60%, 65%, 70%, 75%, 80%, 85%, 90%, 91%, 92%, 93%, 94%, 95%, 96%, 97%, 98%, or 99% identity to the amino acid residues 590 to 825 of SEQ ID NO: 2 or the amino acid residues 595 to 830 of SEQ ID NO: 4.

In one embodiment, the TPS homologs suitable for the present invention may comprise the conserved motifs as shown in the amino acid sequence of SEQ ID NO: 38, 39, 40, 41, 42, 43, 44, 45, 46, 47, 48 and 49.

In further embodiments, the TPS homologs suitable for the present invention may comprise an amino acid sequence having at least 49%, preferably, 50%, 55%, 60%, 65%, 70%, 75%, 80%, 85%, 90%, 91%, 92%, 93%, 94%, 95%, 96%, 97%, 98%, or 99% identity to the amino acid sequence of SEQ ID NO: 2 or 4, wherein the amino acid sequence further comprises the amino acid sequence of SEQ ID NO: 38, 39, 40, 41, 42, 43, 44, 45, 46, 47, 48 and 49.

As used herein, “functional variants,” or “functional equivalent,” of a molecule (e.g., a polypeptide or nucleic acid sequence) is intended to mean a molecule having substantially similar sequence as compared to the non-variant molecule while retaining the activity of the non-variant molecule in whole or in part.

For nucleotide sequences comprising an open reading frame, functional variants include those sequences that, because of the degeneracy of the genetic code, encode the identical amino acid sequence of the native protein. Naturally occurring allelic variants can be identified with the use of well-known molecular biology techniques, such as, for example, with polymerase chain reaction (PCR) and hybridization techniques. Functional variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. A variant nucleotide sequence may also contain insertions, deletions, or substitutions of one or more nucleotides relative to the nucleotide sequence found in nature. Accordingly, a variant protein may contain insertions, deletions, or substitutions of one or more amino acid residues relative the amino acid sequence found in nature. Generally, variants of the nucleotide sequence of SEQ ID NO: 1, 3, 16, 18, 20, 22, 24, 26, 28, 30, 32, 34, 36, 50 or 51 or the amino acid sequence of SEQ ID NO: 2, 4, 17, 19, 21, 23, 25, 27, 29, 31, 33, 35 or 37, will have at least 70%, preferably 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% sequence identity to the corresponding nucleotide or amino acid sequence. The functional variants of the polynucleotide sequence of SEQ ID NO: 1, 3, 16, 18, 20, 22, 24, 26, 28, 30, 32, 34, 36, 50 or 51 may be variants of the corresponding wild-type polynucleotide sequence, provided that they encode a polypeptide retaining the activity of the polypeptide encoded by the polynucleotide sequence of SEQ ID NO: 1, 3, 16, 18, 20, 22, 24, 26, 28, 30, 32, 34, 36, 50 or 51 in conferring an increase content in one or more of protein, oil, or one or more amino acids in a plant, plant cell, or plant part relative to a corresponding wild-type plant, plant cell, or plant part. In some embodiments, such functional variants are capable of conferring increased content in protein and one or more amino acids in a plant, plant cell, or plant part relative to a corresponding wild-type plant, plant cell, or plant part. In other embodiments, such functional variants are capable of conferring increased content in protein, oil and one or more amino acids in a plant, plant cell, or plant part relative to a corresponding wild-type plant, plant cell, or plant part.

Likewise, the functional variants of the amino acid sequence of SEQ ID NO: 2, 4, 17, 19, 21, 23, 25, 27, 29, 31, 33, 35 or 37 may be variants of the corresponding wild-type amino acid sequences, provided that they retain the activity of the protein having the amino acid sequence of SEQ ID NO: 2, 4, 17, 19, 21, 23, 25, 27, 29, 31, 33, 35 or 37 in conferring an increase content in one or more of protein, oil, or one or more amino acids in a plant, plant cell, or plant part relative to a corresponding wild-type plant, plant cell, or plant part. In some embodiments, such functional variants are capable of conferring increased content in protein and one or more amino acids in a plant, plant cell, or plant part relative to a corresponding wild-type plant, plant cell, or plant part. In other embodiment, such functional variants are capable of conferring increased content in protein, oil and one or more amino acids in a plant, plant cell, or plant part relative to a corresponding wild-type plant, plant cell, or plant part. Moreover, in addition to the TPS homologs shown in SEQ ID NO: 1, 3, 16, 18, 20, 22, 24, 26, 28, 30, 32, 34 or 36, which encode the polypeptide of SEQ ID NO: 2, 4, 17, 19, 21, 23, 25, 27, 29, 31, 33, 35 or 37, respectively, the skilled worker will recognize that DNA sequence polymorphisms which lead to changes in the amino acid sequences of SEQ ID NO: 2, 4, 17, 19, 21, 23, 25, 27, 29, 31, 33, 35 or 37 may exist naturally within a population. These genetic polymorphisms in the polynucleotide sequence of SEQ ID NO: 1, 3, 16, 18, 20, 22, 24, 26, 28, 30, 32, 34 or 36 may exist between individuals within a population owing to natural variation. These natural variants usually bring about a variance of 1 to 5% in the nucleotide sequence of SEQ ID NO: 1, 3, 16, 18, 20, 22, 24, 26, 28, 30, 32, 34 or 36. Each and every one of these nucleotide variations and resulting amino acid polymorphisms in the amino acid sequences of SEQ ID NO: 2, 4, 17, 19, 21, 23, 25, 27, 29, 31, 33, 35 or 37, which are the result of natural variation and do not modify the functional activity are to be encompassed by the invention.

In another embodiment, TPS homologs comprise a PF00982.15 Pfam domain having at least 50%, preferably 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% sequence identity to amino acid residues 57 to 541 of SEQ ID NO: 2 or amino acid residues 59 to 546 of SEQ ID NO: 4, and a PF02358.10 Pfam domain having at least 50%, preferably 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% sequence identity to amino acid residues 590 to 825 of SEQ ID NO: 2 or amino acid residues 595 to 830 of SEQ ID NO: 4.

As used herein, “sequence identity” or “identity” refers to a relationship between two or more polynucleotide or polypeptide sequences, as determined by aligning the sequences for maximum correspondence over a specified comparison window. As used in the art, “identity” also means the degree of sequence relatedness between polynucleotide or polypeptide sequences as determined by the match between strings of such sequences.

“Percent identity” (% identity) or “percent sequence identity” (% sequence identity) as used herein refers to the value determined by comparing two optimally aligned sequences over a specified comparison window.

The identity of protein sequences shown in Tables 1 and 2 was determined by pairwise alignment of the sequences over in each case the entire sequence length, using the algorithm of Needleman and Wunsch, as implemented in the The European Molecular Biology Open Software Suite (EMBOSS), version 6.3.1.2 (Trends in Genetics 16 (6), 276 (2000)). Parameters used were Matrix=EBLOSUM62; gapopen=10.0; gapextend=2.0.

Multiple protein alignments and derived dendograms were produced by using the clustal algorithm as implemented in AlignX (version 31 Jul. 2006), a component of the Vector NTI Advance 10.3.0 software package of the Invitrogen Corporation. Parameters used for multiple alignments were default parameters, using gap opening penalty=10; gap extension penalty=0.05; gap separation penalty range=8; matrix=blosum62. The clustal algorithm is publicly available from various sources, e.g. from the ftp server of the European Bioinformaties Institute (EBI) (ebi.ac.uk/pub/software/).

For identification of domains in the sequences of this application, the PFAM-A database release 25.0 was used, which is publicly available (e.g. from pfam.sanger.ac.uk/). Domains were identified by using the hmmscan algorithm. This algorithm is part of the HMMER3 software package and is publicly available (e.g. from the Howard Hughes Medical Institute, Janelia Farm Research Campus (hmmer.org/). Parameters for the hmmscan algorithm were default parameters as implemented in hmmscan (HMMER release 3.0). Domains were scored to be present in a given sequence when the reported E-value was 0.1 or lower and if at least 80% of the length of the PFAM domain model was covered in the algorithm-produced alignment.

Sequence alignments and calculation of percent sequence identity may also be performed with CLUSTAL (see website at ebi.ac.uk/Tools/clustalw2/index.html), the program PileUp (Feng et al., J. Mol. Evolution., 1987, 25:351-360; Higgins et al., CABIOS, 1989, 5:151-153), or the programs Gap and BestFit (Needleman and Wunsch, J. Mol. Biol., 1970, 48:443-453; Smith and Waterman, Adv. Appl. Math., 1981, 2:482-489), which are part of the GCG software packet (Gentics Computer Group, 575 Science Drive, Madison, Wis.).

Other methods and software programs for sequence comparison and alignment and calculation of percent sequence identity are well known in the art. For example, the percent sequence identity may be determined with the Vector NTI Advance 10.3.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (e.g., Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide. Sequence alignments and calculation of percent sequence identity may also be performed with CLUSTAL (see website at ebi.ac.uk/Tools/clustalw2/index.html), the program PileUp (Feng et al., J. Mol. Evolution., 1987, 25:351-360; Higgins et al., CABIOS, 1989, 5:151-153), or the programs Gap and BestFit (Needleman and Wunsch, J. Mol. Biol., 1970, 48:443-453; Smith and Waterman, Adv. Appl. Math., 1981, 2:482-489), which are part of the GCG software packet (Genetics Computer Group, 575 Science Drive, Madison, Wis.).

Methods of identifying homologous sequences with sequence similarity to a reference sequence are known in the art. For example, software for performing BLAST analyses for identification of homologous sequences is publicly available through the National Center for Biotechnology Information (see website at ncbi.nlm.nih.gov). PSI-BLAST (in BLAST 2.0) can also be used to perform an iterated search that detects distant relationships between molecules. When utilizing BLAST or PSI-BLAST, the default parameters of the respective programs (e.g., BLASTN for nucleotide sequences, BLASTX for proteins) can be used (see ncbi.nlm.nih.gov website). Alignment may also be performed manually by inspection. These methods may be used, for example, to identify homologs or variants of the amino acid sequence of SEQ ID NO: 2, 4, 17, 19, 21, 23, 25, 27, 29, 31, 33, 35 or 37, and/or the corresponding coding nucleotide sequences for the use in the expression cassette of the invention.

Nucleic acid molecules encoding functional variants, homologs, analogs, and orthologs of polypeptides can be isolated. The polynucleotides encoding the respective polypeptides or primers based thereon can be used as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

Accordingly, in one embodiment, the nucleic acid molecule to be included in the expression cassette of the invention comprises:
(i) the nucleotide sequence of SEQ ID NO: 1, SEQ ID NO: 3, SEQ ID NO: 50 or SEQ ID NO: 51;
(ii) a nucleotide sequence encoding the amino acid sequence of SEQ ID NO: 2 or SEQ ID NO: 4;
(iii) a nucleotide sequence having at least 70% sequence identity to the nucleotide sequence of SEQ ID NO: 1, SEQ ID NO: 3, SEQ ID NO: 50 or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2 or SEQ ID NO: 4 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% identity to amino acid residues 57 to 541 of SEQ ID NO: 2 or the amino acid residues 59 to 546 of SEQ ID NO: 4, and wherein the Pfam:PF02358.10 trehalose-phosphatase domain has at least 55% identity to the amino acid residues 590 to 825 of SEQ ID NO: 2 or the amino acid residues 595 to 830 of SEQ ID NO: 4; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49,
   and confers an increase in one or more of protein, oil, or one or more amino acids in a plant, plant cell, or plant part relative to a corresponding wild-type plant, plant cell, or plant part.

In another embodiment, the nucleic acid molecule to be included in the expression cassette of the invention comprises:
(i) the nucleotide sequence of SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 36;
(ii) a nucleotide sequence encoding the amino acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, or SEQ ID NO: 36 and encoding a polypeptide having a Pfam:PF00982.15 glycosyltransferase family 20 domain and a Pfam:PF02358.10 trehalose-phosphatase domain; or
(iv) a nucleotide sequence encoding an amino acid sequence having at least 80% sequence identity to the amino acid sequence of SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain,
   and confers an increase in one or more of protein, oil, or one or more amino acids in a plant, plant cell, or plant part relative to a corresponding wild-type plant, plant cell, or plant part.

In a preferred embodiment, the nucleic acid molecule to be included in the expression cassette of the invention comprises a nucleotide sequence encoding a Class II trehalose-6-phosphate synthase. Preferably, the nucleic acid molecule to be included in the expression cassette of the invention confers an increase in protein and one or more amino acids in a plant, plant cell, or plant part relative to a corresponding wild-type plant, plant cell, or plant part. In another embodiment, the nucleic acid molecule to be included in the expression cassette of the invention confers an increase in oil and one or more amino acids in a plant, plant cell, or plant part relative to a corresponding wild-type plant, plant cell, or plant part. More preferably, the nucleic acid molecule to be included in the expression cassette of the invention confers an increase in protein, oil, and one or more amino acids in a plant, plant cell, or plant part relative to a corresponding wild-type plant, plant cell, or plant part.

In a further embodiment, the Pfam:PF00982.15 glycosyltransferase family 20 domain comprises amino acid residues 57 to 541 of SEQ ID NO: 2 or amino acid residues 59 to 546 of SEQ ID NO: 4 and the Pfam:PF02358.10 trehalose-phosphatase domain comprises amino acid residues 590 to 825 of SEQ ID NO: 2 or amino acid residues 595 to 830 of SEQ ID NO: 4.

The term “homolog(s)” is a generic term used in the art to indicate a polynucleotide or polypeptide sequence possessing a high degree of sequence relatedness to a reference sequence. Such relatedness may be quantified by determining the degree of identity and/or similarity between the two sequences. Falling within this generic term are the terms “ortholog(s)” and “paralog(s).” The term “ortholog(s)” refers to a homologous polynucleotide or polypeptide in different organisms due to ancestral relationship of these genes. The term “paralog(s)” refers to a homologous polynucleotide or polypeptide that results from one or more gene duplications within the genome of a species. TPS orthologs, paralogs or homologs may be identified or isolated from the genome of any desired organism, preferably from another plant, according to well known techniques based on their sequence similarity to, for example, the TPS homolog open reading frame having the polynucleotide sequence of SEQ ID NO: 1, 3, 16, 18, 20, 22, 24, 26, 28, 30, 32, 34, 36, 50, or 51, e.g., hybridization, PCR, or computer generated sequence comparisons. For example, all or a portion of a particular open reading frame can be used as a probe that selectively hybridizes to other gene sequences present in a population of cloned genomic DNA fragments (i.e. genomic libraries) from a chosen source organism. Further, suitable genomic libraries may be prepared from any cell or tissue of an organism. Such techniques include hybridization screening of plated DNA libraries (either plaques or colonies; see, e.g., Sambrook, 1989, Molecular Cloning: A Laboratory Manual, 2^{nd} ed., Cold Spring Harbor Laboratory, Cold Spring Harbor Laboratory Press, Cold Spring Harbor, N.Y.) and amplification by PCR using oligonucleotide primers preferably corresponding to sequence domains conserved among related polypeptide or subsequences of the nucleotide sequences provided herein. These methods are known and particularly well suited to the isolation of gene sequences from organisms closely related to the organism from which the probe sequence is derived. The application of these methods using all or a portion of an open reading frame of a TPS homolog as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are known in the art.

Suitable oligonucleotides for use as primers in probing or amplification reactions as the PCR reaction described above, may be about 30 or fewer nucleotides in length (e.g., 9, 12, 15, 18, 20, 21, 22, 23, or 24, or any number between 9 and 30). Generally, specific primers are upwards of 14 nucleotides in length. For optimum specificity and cost effectiveness, primers of 16 to 24 nucleotides in length are preferred. Those skilled in the art are well versed in the design of primers for use in processes such as PCR. If required, probing can be done with entire restriction fragments of the genes disclosed herein which may be 100's or even 1000's of nucleotides in length.

A TPS homolog may also readily be identified by searching in specialized databases containing conserved protein domains such as Pfam (Finn et al. Nucleic Acids Research (2006) Database Issue 34:D247-D251). The Pfam database compiles a large collection of multiple sequence alignments and hidden Markov models (HMM) covering many common protein domains and families and is available through the Sanger Institute in the United Kingdom (Bateman et al., Nucleic Acids Research 30(1): 276-280 (2002)). Tools useful in searching such databases are well known in the art, for example INTERPRO (European Bioinformatics institute, UK) which allows searching several protein domain databases simultaneously. The amino acid positions of two Pfam domains in the sequences of various TPS homologs are provided in Table 2 above.

Nucleotide sequences may be codon optimized to improve expression in heterologous host cells. Nucleotide sequences from a heterologous source are codon optimized to match the codon bias of the host. A codon consists of a set of three nucleotides, referred to as a triplet, which encodes a specific amino acid in a polypeptide chain or for the termination of translation (stop codons). The genetic code is redundant in that multiple codons specify the same amino acid, i.e., 61 codons encoding for 20 amino acids. Organisms exhibit preference for one of the several codons encoding the same amino acid, which is known as codon usage bias. The frequency of codon usage for different species has been determined and recorded in codon usage tables. Codon optimization replaces infrequently used codons present in a DNA sequence of a heterologous gene with preferred codons of the host, based on a codon usage tables. The amino acid sequence is not altered during the process. Codon optimization can be performed using gene optimization software, such as Leto 1.0 from Entelechon. Protein sequences for the genes to be codon optimized are back-translated in the program and the codon usage is selected from a list of organisms. Leto 1.0 replaces codons from the original sequence with codons that are preferred by the organism into which the sequence will be transformed. The DNA sequence output is translated and aligned to the original protein sequence to ensure that no unwanted amino acid changes were introduced. For example, the nucleotide sequence of SEQ TD NO: 50 is the codon optimized version of the nucleotide sequence of SEQ ID NO: 1 for expression in maize. As a further example, the nucleotide sequence of SEQ ID NO: 51 is the codon optimized version of the nucleotide sequence of SEQ ID NO: 3 for expression in maize.

In addition to codon optimization of a sequence from a heterologous source, gene optimization entails further modifications to the DNA sequence to optimize the gene sequence for expression without altering the protein sequence. The Leto 1.0 program can also be used to remove sequences that might negatively impact gene expression, transcript stability, protein expression or protein stability, including but not limited to, transcription splice sites, DNA instability motifs, plant polyadenylation sites, secondary structure, AU-rich RNA elements, secondary ORFs, codon tandem repeats, long range repeats. This can also be done to optimize gene sequences originating from the host organism. Another component of gene optimization is to adjust the G/C content of a heterologous sequence to match the average G/C content of endogenous genes of the host.

For example, to provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) eliminate sequences that cause destabilization, inappropriate polyadenylation, degradation and termination of RNA, or that form secondary structure hairpins or RNA splice sites; or 5) eliminate antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

In some embodiments of the invention, the nucleic acid molecule encoded by the transgene is codon optimized. The nucleic acid sequence may be codon optimized for any host cell in which it is expressed. In one embodiment, the nucleic acid sequence is codon optimized for maize. In further embodiments, the nucleic acid sequence may also be codon optimized for other plant species including, but not limited to rice, wheat, barley, soybean, canola, rapeseed, cotton, sugarcane, or alfalfa.

1.2 Other Regulatory Elements

In addition to the promoter and the nucleic acid molecule encoding a TPS homolog, the expression cassettes of the invention may further comprise other regulatory elements. The term “regulatory elements” encompasses all sequences which may influence construction or function of the expression cassette. Regulatory elements may, for example, modify transcription and/or translation in prokaryotic or eukaryotic organism. Thus, the expression profile of the nucleic acid molecule included in the aforementioned expression cassettes may be modulated depending on the combination of the transcription regulating nucleotide sequence and the other regulatory element(s) comprised in the expression cassette.

In one embodiment, the aforementioned expression cassettes may further comprise at least one additional regulatory element selected from the group consisting of:
(a) 5′-untranslated regions (or 5′-UTR),
(b) intron sequences,
(c) transcription termination sequences (or terminators).
   In another embodiment, the aforementioned expression cassettes may further comprise a protein targeting sequence.

A variety of 5′ and 3′ transcriptional regulatory sequences are available for use in the expression cassettes of the present invention. As the DNA sequence between the transcription initiation site and the start codon of the coding sequence, i.e., the 5′-untranslated sequence, can influence gene expression, one may wish to include a particular 5′-untranslated sequence in the expression cassettes of the invention. Preferred 5′-untranslated sequences include those sequences predicted to direct optimum expression of the attached gene, i.e., consensus 5′-untranslated sequences which may increase or maintain mRNA stability and prevent inappropriate initiation of translation. The choice of such sequences will be known to those of skill in the art. Sequences that are obtained from genes that are highly expressed in plants will be most preferred. Also preferred is the 5′-untranslated region obtained from the same gene as the transcription regulating sequence to be included in the expression cassette of the invention.

Additionally, it is known in the art that a number of non-translated leader sequences are capable of enhancing expression, for example, leader sequences derived from viruses. For example, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie 1987; Skuzeski 1990). Other viral leader sequences known in the art include, but not limited to, Picornavirus leaders, for example, EMCV leader (Encephalomyocarditis 5′ noncoding region) (Elroy-Stein 1989), Potyvirus leaders, for example, TEV leader (Tobacco Etch Virus), MDMV leader (Maize Dwarf Mosaic Virus), Human immunoglobulin heavy-chain binding protein (BiP) leader (Macejak 1991), and untranslated leader from the coat protein mRNA of alfalfa mosaic virus (AMV RNA 4) (Jobling 1987).

The 3′ regulatory sequence preferably includes from about 50 to about 1,000, more preferably about 100 to about 1,000, base pairs and contains plant transcriptional and translational termination sequences. Transcription termination sequences, or terminators, are responsible for the termination of transcription and correct mRNA polyadenylation. Thus, the terminators preferably comprise a sequence inducing polyadenylation. The terminator may be heterologous with respect to the transcription regulating nucleotide sequence and/or the nucleic acid sequence to be expressed, but may also be the natural terminator of the gene from which the transcription regulating nucleotide sequence and/or the nucleic acid sequence to be expressed is obtained. In one embodiment, the terminator is heterologous to the transcription regulating nucleotide sequence and/or the nucleic acid sequence to be expressed. In another embodiment, the terminator is the natural terminator of the gene of the transcription regulating nucleotide sequence.

Appropriate terminators and those which are known to function in plants include, but are not limited to, CaMV 35S terminator, the tml terminator, the nopaline synthase (NOS) terminator (SEQ ID NO: 11), the pea rbcS E9 terminator, the terminator for the T7 transcript from the octopine synthase gene of Agrobacterium tumefaciens (SEQ ID NO: 13), the 3′ end of the protease inhibitor I or II genes from potato or tomato, and the TOI3357 terminator from Oiyza sativa (SEQ ID NO: 76). Alternatively, one also could use a gamma coixin, oleosin 3 or other terminator from the genus Coix. Preferred 3′ regulatory elements include, but are not limited to, those from the nopaline synthase (NOS) gene of Agrobacterium tumefaciens (SEQ ID NO: 11) (Bevan 1983), the terminator for the T7 transcript from the octopine synthase gene of Agrobacterium tumefaciens, and the 3′ end of the protease inhibitor I or II genes from potato or tomato. A non-limiting example of a terminator to be included in the expression cassettes of the invention comprises the polynucleotide sequence as described by SEQ ID NO: 11, 13, or 76.

Accordingly, in some preferred embodiments, the expression cassettes of the invention may further comprise a terminator selected from the group consisting of:
(a) a terminator comprising the nucleotide sequence of SEQ ID NO: 11, 13, or 76; and
(b) a terminator comprising a nucleotide sequence having at least 90%, preferably 95%, 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 11, 13, or 76.

Transcription regulatory elements can also include intron sequences that have been shown to enhance gene expression in transgenic plants, particularly in monocotyledonous plants. The intron sequence is preferably inserted in the aforementioned expression cassettes between the transcription regulating nucleotide sequence and the nucleic acid sequence to be expressed. In an expression cassette of the invention comprising an ScBV promoter or a functional fragment thereof, any intron sequence may be used. Preferably, such expression enhancing intron sequences are from monocotyledonous plants. Preferred intron sequences include, but are not limited to, intron sequences from Adh1 (Callis 1987), bronze 1, actin 1, actin 2 (WO 00/760067), the sucrose synthase intron (Vasil 1989) (see The Maize Handbook, Chapter 116, Freeling and Walbot, Eds., Springer, New York, 1994); the Atc17 intron from the ADP-ribosylation factor 1 (ARF1) gene NEENAc17 intron from Arabidopsis thaliana (SEQ ID NO: 74), and the Atss1 intron from the aspartyl protease family protein related NEENA gene intron from Arabidopsis thaliana (SEQ ID NO: 75). More preferably, the intron sequences are:

(a) the introns of rice Metallothionin 1 gene, preferably intron I thereof, most preferably the intron sequence as described by SEQ ID NO: 10,

(b) the introns of the Zea mays ubiquitin gene, preferably intron I thereof, most preferably the intron sequence as described by SEQ ID NO: 52,

(c) the introns of the rice actin gene, preferably intron I thereof, most preferably the intron sequence as described by nucleotide 121 to 568 of the sequence described by GenBank Accession No. X63830, and

(d) the introns of the Zea mays alcohol dehydrogenase (adh) gene, preferably intron 6 thereof, most preferably the intron sequence as described by nucleotide 3,135 to 3,476 of the sequence described by GenBank Accession No. X04049.

Accordingly, in some preferred embodiments, the expression cassettes of the invention may further comprise the intron of the rice Metallothionin 1 gene comprising the nucleotide sequence of SEQ ID NO: 10 or a nucleotide sequence having at least 90%, preferably 95%, 96%, 97%, 98%, 99%, 99.1%, 99.2%, 99.3%, 99.4%, 99.5%, 99.6%, 99.7%, 99.8%, or 99.9% identity to the nucleotide sequence of SEQ ID NO: 10; and

Isolation of rice Metallothionein) introns and functional variants thereof are described for example in US 2009/0144863 (hereby incorporated by reference in its entirety). Additional intron sequences with expression enhancing properties in plants may also be identified and isolated according to the disclosure of US 2006/0094976 (hereby incorporated by reference in its entirety).

1.3 Protein Targeting Sequences

In addition to the aforementioned components, the expression cassettes of the present invention may further comprise protein targeting sequences. The term “protein targeting sequences” as used herein encompasses all nucleotide sequences encoding transit peptides for directing a protein to a particular cell compartment such as vacuole, nucleus, all types of plastids like amyloplasts, chloroplasts, or chromoplasts, extracellular space, mitochondria, endoplasmic reticulum, oil bodies, peroxisomes and other compartments of plant cells (for review see Kermode 1996, Crit. Rev. Plant Sci. 15: 285-423 and references cited therein).

In some embodiments, it may be desirable for the TPS homolog polypeptide to be targeted to a particular cell compartment such as a plastid. To do so, a plastid transit peptide may be used. Nucleotide sequences encoding plastid transit peptides are well known in the art, as disclosed, for example, in U.S. Pat. Nos. 5,717,084; 5,728,925; 6,063,601; 6,130,366; and the like. Cell compartment transit peptides include, but are not limited to, the ferredoxin transit peptide and the starch branching enzyme 2b transit peptide. In a preferred embodiment the transit peptide is a plastid-targeting peptide from a ferredoxin gene from Silene pratensins (SpFdx) (for example, SEQ ID NO: 5 or SEQ ID NO: 73, each encoding SEQ ID NO: 6). SpFdx and several of its variants have been shown to effectively target polypeptides to the stroma (Pilon, et al., 1995, J Biol. Chem. 270(8):3882-93).

Accordingly, in some preferred embodiments, the expression cassettes of the invention may further comprise at least one heterologous nucleotide sequence encoding a transit peptide to target the TPS homolog to a plastid, wherein the nucleotide sequence encoding the plastid-targeting transit peptide comprises:

(a) the nucleotide sequence of SEQ ID NO: 5 or 73;

(b) a nucleotide sequence having at least 95%, preferably 96%, 97%, 98%, 99%, 99.1%, 99.2%, 99.3%, 99.4%, 99.5%, 99.6%, 99.7%, 99.8%, or 99.9% identity to the sequence of SEQ ID NO: 5 or 73;

(c) a nucleotide sequence encoding the amino acid sequence of SEQ ID NO: 6; or

(d) a nucleotide sequence encoding a peptide having at least 95%, preferably 96%, 97%, 98%, 99%, 99.1%, 99.2%, 99.3%, 99.4%, 99.5%, 99.6%, 99.7%, 99.8%, or 99.9% identity to the amino acid sequence of SEQ ID NO: 6.

1.4 Preferred Embodiments of Expression Cassettes

It is found that, by expressing certain TPS homologs in a plant, plant cell, or plant part under control of some specific types of promoters, optionally in combination with other specific types of regulatory elements and/or targeting peptides, the content of one or more of protein, oil, or one or more amino acids in such a plant, plant cell, or plant part is surprisingly increased. This section exemplifies some of such preferred expression cassettes of the invention.

In one aspect, the present invention provides expression cassette (I) comprising:

(a) a promoter that is functional in a plant as disclosed in Section 1.1.1;

(b) a nucleic acid molecule encoding a TPS homolog as disclosed in Section 1.1.2; and

(c) a rice intron as disclosed in Section 1.2.

In another aspect, the present invention provides expression cassette (II) comprising:

(a) a constitutive promoter as disclosed in Section 1.1.1;

(b) a nucleic acid molecule encoding a TPS homolog as disclosed in Section 1.1.2; and

(c) an intron as disclosed in Section 1.2.

In yet another aspect, the present invention provides expression cassette (III) comprising:

(a) a promoter that is functional in a plant as disclosed in Section 1.1.1; and

(b) a nucleic acid molecule encoding a TPS homolog as disclosed in Section 1.1.2,

wherein expression of the nucleic acid molecule in a plant, plant cell, or plant part confers increased content of protein and one or more amino acids in said plant, plant cell, or plant part relative to a corresponding wild-type plant, plant cell, or plant part.

Preferably, the nucleic acid molecule encoding a TPS homolog to be included in the aforementioned expression cassettes (I), (II) and (III) of the invention comprises:
(a) the nucleotide sequence of SEQ ID NO: 1, 3, 16, 18, 20, 22, 24, 26, 28, 30, 32, 34, 36, 50, or 51;
(b) a nucleotide sequence encoding the amino acid sequence of SEQ ID NO: 2, 4, 17, 19, 21, 23, 25, 27, 29, 31, 33, 35 or 37;
(c) a nucleotide sequence having at least 70% sequence identity to the nucleotide sequence of SEQ ID NO: 1, 3, 16, 18, 20, 22, 24, 26, 28, 30, 32, 34, 36, 50, or 51 and encoding a polypeptide having a Pfam:PF00982.15 glycosyltransferase family 20 domain and a Pfam:PF02358.10 trehalose-phosphatase domain;
(d) a nucleotide sequence encoding an amino acid sequence having at least 80% sequence identity to the amino acid sequence of SEQ ID NO: 2, 4, 17, 19, 21, 23, 25, 27, 29, 31, 33, 35, or 37 and having a Pfam:PF00982.15 glycosyltransferase family 20 domain and a Pfam:PF02358.10 trehalose-phosphatase domain; or
(e) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% identity to amino acid residues 57 to 541 of SEQ ID NO: 2 or the amino acid residues 59 to 546 of SEQ ID NO: 4, and wherein the Pfam:PF02358.10 trehalose-phosphatase domain has at least 55% identity to the amino acid residues 590 to 825 of SEQ ID NO: 2 or the amino acid residues 595 to 830 of SEQ ID NO: 4.

More preferably, the nucleic acid molecule encoding a TPS homolog to be included in the aforementioned expression cassettes (I) and (II) of the invention comprises:

(a) the nucleotide sequence of SEQ ID NO: 1, 3, 16, 18, 20, 22, 24, 26, 28, 30, 32, 34, 36, 50, or 51;

(b) a nucleotide sequence encoding the amino acid sequence of SEQ ID NO: 2, 4, 17, 19, 21, 23, 25, 27, 29, 31, 33, 35 or 37;

(c) a nucleotide sequence having at least 95% identity to the nucleotide sequence of SEQ ID NO: 1, 3, 16, 18, 20, 22, 24, 26, 28, 30, 32, 34, 36, 50, or 51; or

(d) a nucleotide sequence encoding an amino acid sequence having at least 95% identity to the amino acid sequence of SEQ ID NO: 2, 4, 17, 19, 21, 23, 25, 27, 29, 31, 33, 35, or 37.

In another aspect, the present invention provides expression cassette (IV) comprising:

(a) a promoter that is functional in a plant as disclosed in Section 1.1.1;

(b) a nucleic acid molecule encoding a TPS homolog as disclosed in Section 1.1.2; and

(c) the first intron of the rice Metallothionin 1 gene as disclosed in Section 1.2.

In a further aspect, the present invention provides expression cassette (V) comprising:

(a) a constitutive promoter that is functional in a plant as disclosed in Section 1.1.1;

(b) a nucleic acid molecule encoding a TPS homolog; and

(c) an intron,

wherein the constitutive promoter comprises:

(i) the nucleotide sequence of SEQ ID NO: 8 or 9;

(ii) a nucleotide sequence having at least 95% identity to the nucleotide sequence of SEQ ID NO: 8 or 9, wherein said nucleotide sequence has constitutive expression activity; or

(iii) a fragment of the nucleotide sequence of SEQ ID NO: 8 or 9, wherein the fragment has constitutive expression activity.

Preferably, the nucleic acid molecule encoding a TPS homolog to be included in the aforementioned expression cassettes (IV) and (V) of the invention comprises:

(a) the nucleotide sequence of SEQ ID NO: 1, 3, 16, 18, 20, 22, 24, 26, 28, 30, 32, 34, 36, 50, or 51;

(b) a nucleotide sequence encoding the amino acid sequence of SEQ ID NO: 2, 4, 17, 19, 21, 23, 25, 27, 29, 31, 33, 35, or 37;

(c) a nucleotide sequence having at least 70% identity to the nucleotide sequence of SEQ ID NO: 1, 3, 16, 18, 20, 22, 24, 26, 28, 30, 32, 34, 36, 50, or 51; or

(d) a nucleotide sequence encoding an amino acid sequence having at least 80% identity to the amino acid sequence of SEQ ID NO: 2, 4, 17, 19, 21, 23, 25, 27, 29, 31, 33, 35, or 37.

In some embodiments, the intron to be included in the aforementioned expression cassettes (I)-(V) of the invention is an intron of the rice Metallothionin 1 gene, preferably, comprising the nucleotide sequence of SEQ ID NO: 10 or a nucleotide sequence having at least 90% identity to the nucleotide sequence of SEQ ID NO: 10.

Optionally, the aforementioned expression cassettes of the invention further comprise a heterologous nucleotide sequence encoding a transit peptide targeting the TPS homolog to a plastid as disclosed in Section 1.3. For example, in one embodiment, the expression cassette comprises a promoter that is functional in a plant as disclosed in Section 1.1.1, a nucleic acid molecule

The aforementioned expression cassettes of the invention may also optionally comprise a terminator as disclosed in Section 1.2.

Accordingly, examples of the expression cassettes of the invention may include, but are not limited to, the various combinations of the nucleotide components as exemplified in Table 3 below.

TABLE 3
Examples of the expression cassettes of the invention.

| Promoter | Intron | Targeting peptide | Gene | Terminator |
|---|---|---|---|---|
| Embryo-specific | An intron of rice | Plastid-targeting | TPS homolog (e.g. | t-NOS (e.g. SEQ |
| or preferential (e.g. | Met1 (e.g. SEQ ID | peptide (e.g. SEQ | SEQ ID NO: 1, 3, | ID NO: 11) or t- |
| SEQ ID NO: 7) | NO: 10) or an | ID NO: 5 or 73) | 16, 18, 20, 22, | OCS3 (e.g. SEQ |
| | intron of rice | | 24, 26, 28, 30, | ID NO: 13) |
| | MADS3 (e.g. SEQ | | 32, 34, 36, 50, or 51) | |
| | ID NO: 12) | | | |
| Embryo-specific | An intron of rice | None | TPS homolog (e.g. | t-NOS (e.g. SEQ |
| or preferential (e.g. | Met1 (e.g. SEQ ID | | SEQ ID NO: 1, 3, | ID NO: 11) or t- |
| SEQ ID NO: 7) | NO: 10) or an | | 16, 18, 20, 22, | OCS3 (e.g. SEQ |
| | intron of rice | | 24, 26, 28, 30, | ID NO: 13) |
| | MADS3 (e.g. SEQ | | 32, 34, 36, 50, or 51) | |
| | ID NO: 12) | | | |
| Embryo-specific | None | Plastid-targeting | TPS homolog (e.g. | t-NOS (e.g. SEQ |
| or preferential (e.g. | | peptide (e.g. SEQ | SEQ ID NO: 1, 3, | ID NO: 11) or t- |
| SEQ ID NO: 7) | | ID NO: 5 or 73) | 16, 18, 20, 22, | OCS3 (e.g. SEQ |
| | | | 24, 26, 28, 30, | ID NO: 13) |
| | | | 32, 34, 36, 50, or 51) | |
| Embryo-specific | None | None | TPS homolog (e.g. | t-NOS (e.g. SEQ |
| or preferential (e.g. | | | SEQ ID NO: 1, 3, | ID NO: 11) or t- |
| SEQ ID NO: 7) | | | 16, 18, 20, 22, | OCS3 (e.g. SEQ |
| | | | 24, 26, 28, 30, | ID NO: 13) |
| | | | 32, 34, 36, 50, or 51) | |
| Whole-seed | An intron of rice | Plastid-targeting | TPS homolog (e.g. | t-NOS (e.g. SEQ |
| specific or | Met1 (e.g. SEQ ID | peptide (e.g. SEQ | SEQ ID NO: 1, 3, | ID NO: 11) or t- |
| preferential (e.g. | NO: 10) or an | ID NO: 5 or 73) | 16, 18, 20, 22, | OCS3 (e.g. SEQ |
| SEQ ID NO: 69 or | intron of rice | | 24, 26, 28, 30, | ID NO: 13) |
| 14 or 77) | MADS3 (e.g. SEQ | | 32, 34, 36, 50, or 51) | |
| | ID NO: 12) | | | |
| Whole-seed | An intron of rice | None | TPS homolog (e.g. | t-NOS (e.g. SEQ |
| specific or | Met1 (e.g. SEQ ID | | SEQ ID NO: 1, 3, | ID NO: 11) or t- |
| preferential (e.g. | NO: 10) or an | | 16, 18, 20, 22, | OCS3 (e.g. SEQ |
| SEQ ID NO: 69 or | intron of rice | | 24, 26, 28, 30, | ID NO: 13) |
| 14 or 77) | MADS3 (e.g. SEQ | | 32, 34, 36, 50, or 51) | |
| | ID NO: 12) | | | |
| Whole-seed | None | Plastid-targeting | TPS homolog (e.g. | t-NOS (e.g. SEQ |
| specific or | | peptide (e.g. SEQ | SEQ ID NO: 1, 3, | ID NO: 11) or t- |
| preferential (e.g. | | ID NO: 5 or 73) | 16, 18, 20, 22, | OCS3 (e.g. SEQ |
| SEQ ID NO: 69 or | | | 24, 26, 28, 30, | ID NO: 13) |
| 14 or 77) | | | 32, 34, 36, 50, or | |
| | | | 51) | |
| Whole-seed | None | None | TPS homolog (e.g. | t-NOS (e.g. SEQ |
| specific or | | | SEQ ID NO: 1, 3, | ID NO: 11) or t- |
| preferential (e.g. | | | 16, 18, 20, 22, | OCS3 (e.g. SEQ |
| SEQ ID NO: 69 or | | | 24, 26, 28, 30, | ID NO: 13) |
| 14 or 77) | | | 32, 34, 36, 50, or 51) | |
| Endosperm | An intron of rice | Plastid-targeting | TPS homolog (e.g. | t-NOS (e.g. SEQ |
| specific or | Met1 (e.g. SEQ ID | peptide (e.g. SEQ | SEQ ID NO: 1, 3, | ID NO: 11) or t- |
| preferential (e.g. | NO: 10) or an | ID NO: 5 or 73) | 16, 18, 20, 22, | OCS3 (e.g. SEQ |
| SEQ ID NO: | intron of rice | | 24, 26, 28, 30, | ID NO: 13) |
| 15 or 71) | MADS3 (e.g. SEQ | | 32, 34, 36, 50, or 51) | |
| | ID NO: 12) | | | |
| Endosperm | An intron of rice | None | TPS homolog (e.g. | t-NOS (e.g. SEQ |
| specific or | Met1 (e.g. SEQ ID | | SEQ ID NO: 1, 3, | ID NO: 11) or t- |
| preferential (e.g. | NO: 10) or an | | 16, 18, 20, 22, | OCS3 (e.g. SEQ |
| SEQ ID NO: | intron of rice | | 24, 26, 28, 30, | ID NO: 13) |
| 15 or 71) | MADS3 (e.g. SEQ | | 32, 34, 36, 50, or 51) | |
| | ID NO: 12) | | | |
| Endosperm | None | Plastid-targeting | TPS homolog (e.g. | t-NOS (e.g. SEQ |
| specific or | | peptide (e.g. SEQ | SEQ ID NO: 1, 3, | ID NO: 11) or t- |
| preferential (e.g. | | ID NO: 5 or 73) | 16, 18, 20, 22, | OCS3 (e.g. SEQ |
| SEQ ID NO: | | | 24, 26, 28, 30, | ID NO: 13) |
| 15 or 71) | | | 32, 34, 36, 50, or 51) | |
| Endosperm | None | None | TPS homolog (e.g. | t-NOS (e.g. SEQ |
| specific or | | | SEQ ID NO: 1, 3, | ID NO: 11) or t- |
| preferential (e.g. | | | 16, 18, 20, 22, | OCS3 (e.g. SEQ |
| SEQ ID NO: | | | 24, 26, 28, 30, | ID NO: 13) |
| 15 or 71) | | | 32, 34, 36, 50, or 51) | |
| Constitutive (e.g. | An intron of rice | Plastid-targeting | TPS homolog (e.g. | t-NOS (e.g. SEQ |
| SEQ ID NO: 8 or | Met1 (e.g. SEQ ID | peptide (e.g. SEQ | SEQ ID NO: 1, 3, | ID NO: 11) or t- |
| 9 or 70) | NO: 10) or an | ID NO: 5 or 73) | 16, 18, 20, 22, | OCS3 (e.g. SEQ |
| | intron of rice | | 24, 26, 28, 30, | ID NO: 13) |
| | MADS3 (e.g. SEQ | | 32, 34, 36, 50, or 51) | |
| | ID NO: 12) | | | |
| Constitutive (e.g. | An intron of rice | None | TPS homolog (e.g. | t-NOS (e.g. SEQ |
| SEQ ID NO: 8 or | Met1 (e.g. SEQ ID | | SEQ ID NO: 1, 3, | ID NO: 11) or t- |
| 9 or 70) | NO: 10) or an | | 16, 18, 20, 22, | OCS3 (e.g. SEQ |
| | intron of rice | | 24, 26, 28, 30, | ID NO: 13) |
| | MADS3 (e.g. SEQ | | 32, 34, 36, 50, or 51) | |
| | ID NO: 12) | | | |
| Constitutive (e.g. | None | Plastid-targeting | TPS homolog (e.g. | t-NOS (e.g. SEQ |
| SEQ ID NO: 8 or | | peptide (e.g. SEQ | SEQ ID NO: 1, 3, | ID NO: 11) or t- |
| 9 or 70) | | ID NO: 5 or 73) | 16, 18, 20, 22, | OCS3 (e.g. SEQ |
| | | | 24, 26, 28, 30, | ID NO: 13) |
| | | | 32, 34, 36, 50, or 51) | |
| Constitutive (e.g. | None | None | TPS homolog (e.g. | t-NOS (e.g. SEQ |
| SEQ ID NO: 8 or | | | SEQ ID NO: 1, 3, | ID NO: 11) or t- |
| 9 or 70) | | | 16, 18, 20, 22, | OCS3 (e.g. SEQ |
| | | | 24, 26, 28, 30, | ID NO: 13) |
| | | | 32, 34, 36, 50, or 51) | |

In some embodiments, the expression of the nucleic acid molecule encoding a TPS homolog included in the expression cassettes of the invention in a plant, plant cell, or plant part confers an increase in one or more of protein, oil, or one or more amino acids in said plant, plant cell, or plant part relative to a corresponding wild-type plant, plant cell, or plant part. In one embodiment, the expression of the nucleic acid molecule encoding a TPS homolog confers an increase in protein relative to a corresponding wild-type plant, plant cell, or plant part. In another embodiment, the expression of the nucleic acid molecule encoding a TPS homolog confers an increase in oil relative to a corresponding wild-type plant, plant cell, or plant part. In a further embodiment, the expression of the nucleic acid molecule encoding a TPS homolog confers an increase in one or more amino acids relative to a corresponding wild-type plant, plant cell, or plant part. In a preferred embodiment, the expression of the nucleic acid molecule encoding a TPS homolog confers an increase in protein and one or more amino acids relative to a corresponding wild-type plant, plant cell, or plant part. In another embodiment, the expression of the nucleic acid molecule encoding a TPS homolog confers an increase in oil and one or more amino acids in a plant, plant cell, or plant part relative to a corresponding wild-type plant, plant cell, or plant part. In a more preferred embodiment, the expression of the nucleic acid molecule encoding a TPS homolog confers an increase in protein, oil, and one or more amino acids relative to a corresponding wild-type plant, plant cell, or plant part.

### 2. Recombinant Constructs and Vectors

The aforementioned expression cassettes are preferably comprised in a recombinant construct and/or a vector, preferably a plant transformation vector. Numerous vectors for recombinant DNA manipulation or plant transformation are known to the person skilled in the pertinent art. The selection of vector will depend upon the host cell employed. Similarly, the selection of plant transformation vector will depend upon the preferred transformation technique and the target species for transformation.

2.1 Recombinant Constructs

Another aspect of the invention refers to a recombinant construct comprising at least one of the aforementioned expression cassettes. Preferably, the recombinant construct comprises at least one aforementioned expression cassette comprising other regulatory elements described herein for directing the expression of the nucleic acid sequence comprised in the aforementioned expression cassette in an appropriate host cell. More preferably, the recombinant construct comprises at least one aforementioned expression cassette with at least one terminator. Optionally, or in another embodiment, the recombinant construct may comprise at least one aforementioned expression cassette further comprising at least one expression enhancing sequence such as an intron sequence as exemplified herein, for example, in Section 2.

It is further within the scope of the invention that a recombinant construct may comprise more than one aforementioned expression cassette. It is also to be understood that each expression cassette to be included in the recombinant construct may further comprise at least one regulatory element of the same or different type as described herein.

2.2 Vectors

Another aspect of the invention refers to a vector comprising the aforementioned expression cassette or a recombinant construct derived therefrom. The term “vector,” preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination. The vector encompassing the expression cassettes or recombinant constructs of the invention, preferably, further comprises selectable markers as described below for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion.

Vectors can be introduced into prokaryotic or eukaryotic cells via conventional transformation or transfection techniques. The terms “transformation” and “transfection,” conjugation and transduction, as used in the present context, are intended to comprise a multiplicity of processes known in the art for introducing foreign nucleic acid (e.g., DNA) into a host cell, including, but not limited to, calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., 1989, Cold Spring Harbor Laboratory, Cold Spring Harbor Laboratory Press, Cold Spring Harbor, N.Y.) and other laboratory manuals, such as Methods in Molecular Biology (Gartland and Davey eds., 1995, Vol. 44, Agrobacterium Protocols, Humana Press, Totowa, N.J.). Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host or host cells. Preferably, the vector referred to herein is suitable as a cloning vector, i.e. replicable in microbial systems. Such vectors ensure efficient cloning in bacteria and, preferably, yeasts or fungi and make possible the stable transformation of plants. Examples of suitable vectors include, but not limited to, various binary and co-integrated vector systems which are suitable for the T-DNA-mediated transformation as described herein. These vector systems, preferably, also comprise further cis-regulatory elements as described herein, such as selection markers or reporter genes.

2.3 Vector Elements

Recombinant constructs and the vectors derived therefrom may comprise further functional elements. The term “functional element” is to be understood in the broad sense and means all those elements which have an effect on the generation, multiplication or function of the recombinant constructs, vectors or transgenic organisms according to the invention. Examples of such function elements include, but not limited to, selection marker genes, reporter genes, origins of replication, elements necessary for Agrobacterium-mediated transformation, and multiple cloning sites (MCS).

Selection marker genes are useful to select and separate successfully transformed cells. Preferably, within the method of the invention one marker may be employed for selection in a prokaryotic host, while another marker may be employed for selection in a eukaryotic host, particularly the plant species host. The marker may confer resistance against a biocide, such as antibiotics, toxins, heavy metals, or the like, or may function by complementation, imparting prototrophy to an auxotrophic host. Preferred selection marker genes for plants may include, but not limited to, negative selection markers, positive selection markers, and counter selection markers.

Negative selection markers include markers which confer a resistance to a biocidal compound such as a metabolic inhibitor (e.g., 2-deoxyglucose-6-phosphate, WO 98/45456), antibiotics (e.g., kanamycin, G 418, bleomycin or hygromycin) or herbicides (e.g., phosphinothricin or glyphosate). Especially preferred negative selection markers are those which confer resistance to herbicides. These markers can be used, beside their function as a selection marker, to confer a herbicide resistance trait to the resulting transgenic plant. Examples of negative selection markers include, but not limited to
Phosphinothricin acetyltransferases (PAT; also named Bialophos resistance; bar; de Block et al., EMBO J., 1987, 6:2513-2518; EP 0333033; U.S. Pat. No. 4,975,374);
5-enolpyruvylshikimate-3-phosphate synthase (EPSPS; U.S. Pat. No. 5,633,435) or glyphosate oxidoreductase gene (U.S. Pat. No. 5,463,175) conferring resistance to Glyphosate (N-phosphonomethyl glycine) (Shah et al., Science, 1986, 233:478);
Glyphosate degrading enzymes (Glyphosate oxidoreductase; gox);
Dalapon inactivating dehalogenases (deh);
Sulfonylurea- and imidazolinone-inactivating acetolactate synthases (for example mutated ALS variants with, for example, the S4 and/or Hra mutation);
Bromoxynil degrading nitrilases (bxn);
Kanamycin- or G418-resistance genes (NPTII or NPTI) coding for neomycin phosphotransferases (Fraley et al., Proc. Natl. Acad. Sci. USA, 1983, 80:4803), which expresses an enzyme conferring resistance to the antibiotic kanamycin and the related antibiotics neomycin, paromomycin, gentamicin, and G418;
2-Deoxyglucose-6-phosphate phosphatase (DOGR1-Gene product; WO 98/45456; EP 0807836) conferring resistance against 2-deoxyglucose (Randez-Gil et al., Yeast, 1995, 11:1233-1240);
Hygromycin phosphotransferase (IIPT), which mediates resistance to hygromycin (Vanden Elzen et al., Plant Mol. Biol., 1985, 5:299); and
Dihydrofolate reductase (Eichholtz et al., Somatic Cell and Molecular Genetics, 1987, 13:67-76).

Additional negative selection marker genes of bacterial origin that confer resistance to antibiotics include the aadA gene, which confers resistance to the antibiotic spectinomycin, gentamycin acetyl transferase, streptomycin phosphotransferase (SPT), aminoglycoside-3-adenyl transferase and the bleomycin resistance determinant (Svab et al., Plant Mol. Biol., 1990, 14:197; Jones et al., Mol. Gen. Genet., 1987, 210:86; Hille et al., Plant Mol. Biol., 1986, 7:171; Hayford et al., Plant Physiol., 1988, 86:1216). Other negative selection markers include those confer resistance against the toxic effects imposed by D-amino acids like e.g., D-alanine and D-serine (WO 03/060133; Erikson et al., Nat. Biotechnol., 2004, 22(4):455-458), the daol gene encoding a D-amino acid oxidase (EC 1.4.3.3; GenBank Accession No. U60066) from Rhodotorula gracilis (Rhodosporidium toruloides), and the dsdA gene encoding a D-serine deaminase (EC 4.3.1.18; GenBank Accession No. J01603) from E. coli. Depending on the employed D-amino acid, the D-amino acid oxidase markers can be employed as dual function marker offering negative selection (e.g., when combined with for example D-alanine or D-serine) or counter selection (e.g., when combined with D-leucine or D-isoleucine).

Positive selection markers include markers which confer a growth advantage to a transformed plant in comparison with a non-transformed one. Genes like isopentenyltransferase from Agrobacterium tumefaciens (strain PO22; Genbank Accession No. AB025109) may, as a key enzyme of the cytokinin biosynthesis, facilitate regeneration of transformed plants (e.g., by selection on cytokinin-free medium). Corresponding selection methods are described in Ebinuma et al. (Proc. Natl. Acad. Sci. USA, 2000, 94:2117-2121) and Ebinuma et al. (“Selection of marker-free transgenic plants using the oncogenes (ipt, rol A, B, C) of Agrobacterium as selectable markers,” 2000, in Molecular Biology of Woody Plants, Kluwer Academic Publishers). Additional positive selection markers, which confer a growth advantage to a transformed plant in comparison with a non-transformed one, are described in, for example, EP 0601092. Growth stimulation selection markers may include, but not limited to, β-glucuronidase (in combination with, for example, cytokinin glucuronide), mannose-6-phosphate isomerase (in combination with mannose), UDP-galactose-4-epimerase (in combination with, for example, galactose), wherein mannose-6-phosphate isomerase in combination with mannose is especially preferred.

Counter selection markers are especially suitable to select organisms with defined deleted sequences comprising said marker (Koprek et al., Plant J., 1999, 19(6):719-726). Examples for counter selection marker include, but not limited to, thymidine kinases (TK), cytosine deaminases (Gleave et al., Plant Mol. Biol., 1999, 40(2):223-35; Perera et al., Plant Mol. Biol., 1993, 23(4):793-799; Stougaard, Plant J., 1993, 3:755-761), cytochrome P450 proteins (Koprek et al., Plant J., 1999, 19(6):719-726), haloalkan dehalogenases (Naested, Plant J., 1999, 18:571-576), iaaH gene products (Sundaresan et al., Gene Develop., 1995, 9:1797-1810), cytosine deaminase codA (Schlaman and Hooykaas, Plant J., 1997, 11:1377-1385), and tms2 gene products (Fedoroff and Smith, Plant J., 1993, 3:273-289).

Reporter genes encode readily quantifiable proteins and, via their color or enzyme activity, make possible an assessment of the transformation efficacy, the site of expression or the time of expression. Very especially preferred in this context are genes encoding reporter proteins (Schenborn and Groskreutz, Mol. Biotechnol., 1999, 13(1):29-44) such as the green fluorescent protein (GFP) (Haseloff et al., Proc. Natl. Acad. Sci. USA, 1997, 94(6):2122-2127; Sheen et al., Plant J., 1995, 8(5):777-784; Reichel et al., Proc. Natl. Acad. Sci. USA, 1996, 93(12):5888-5893; Chui et al., Curr. Biol., 1996, 6:325-330; Leffel et al., Biotechniques, 1997, 23(5):912-918; Tian et al., Plant Cell Rep., 1997, 16:267-271; WO 97/41228), chloramphenicol transferase, a luciferase (Millar et al., Plant Mol. Biol. Rep., 1992, 10:324-414; Ow et al., Science, 1986, 234:856-859), the aequorin gene (Prasher et al., Biochem. Biophys. Res. Commun., 1985, 126(3):1259-1268), β-galactosidase, R locus gene (encoding a protein which regulates the production of anthocyanin pigments (red coloring) in plant tissue and thus makes possible the direct analysis of the promoter activity without addition of further auxiliary substances or chromogenic substrates; see Dellaporta et al., 1988, In: Chromosome Structure and Function: Impact of New Concepts, 18th Stadler Genetics Symposium, 11:263-282; Ludwig et al., Science, 1990, 247:449), with β-glucuronidase (GUS) being very especially preferred (Jefferson, Plant Mol. Bio. Rep., 1987, 5:387-405; Jefferson et al., EMBO J., 1987, 6:3901-3907). β-glucuronidase (GUS) expression is detected by a blue color on incubation of the tissue with 5-bromo-4-chloro-3-indolyl-β-D-glucoronic acid, bacterial luciferase (LUX) expression is detected by light emission, firefly luciferase (LUC) expression is detected by light emission after incubation with luciferin, and galactosidase expression is detected by a bright blue color after the tissue was stained with 5-bromo-4-chloro-3-indolyl-β-D-galactopyranoside. Reporter genes may also be used as scorable markers as alternatives to antibiotic resistance markers. Such markers can be used to detect the presence or to measure the level of expression of the transferred gene. The use of scorable markers in plants to identify or tag genetically modified cells works well when efficiency of modification of the cell is high. Origins of replication which ensure amplification of the recombinant constructs or vectors according to the invention in, for example, E. coli. Examples of suitable origins of replication include, but not limited to, ORI (origin of DNA replication), the pBR322 ori or the P15A on (Sambrook et al., Molecular Cloning: A Laboratory Manual, 2^{nd} ed., Cold Spring harbor Laboratory, Cold Spring Harbor Laboratory Press, Cold Spring Harbor, N.Y., 1989). Additional examples for replication systems functional in E. coli, are ColE1, pSC101, pACYC184, or the like. In addition to or in place of the E. coli replication system, a broad host range replication system may be employed, such as the replication systems of the P-1 Incompatibility plasmids, e.g., pRK290. These plasmids are particularly effective with armed and disarmed Ti-plasmids for transfer of T-DNA to the plant host.

Other functional elements may be included in the recombinant constructs and the vector derived therefrom of the invention include, but not limited to, other genetic control elements for excision of the inserted sequences from the genome, elements necessary for Agrobacterium-mediated transformation, and multiple cloning sites (MCS).

Other genetic control elements for excision permit removal of the inserted sequences from the genome. Methods based on the cre/lox (Dale and Ow, Proc. Natl. Acad. Sci. USA, 1991, 88:10558-10562; Sauer, Methods, 1998, 14(4):381-392; Odell et al., Mol. Gen. Genet., 1990, 223:369-378), FLP/FRT (Lysnik et al., Nucleic Acid Research, 1993, 21:969-975), or Ac/Ds system (Lawson et al., Mol. Gen. Genet., 1994, 245:608-615; Wader et al., in Tomato Technology (Alan R. Liss, Inc.), 1987, pp. 189-198; U.S. Pat. No. 5,225,341; Baker et al., EMBO J., 1987, 6:1547-1554) permit removal of a specific DNA sequence from the genome of the host organism, if appropriate, in a tissue-specific and/or inducible manner. In this context, the control sequences may mean the specific flanking sequences (e.g., lox sequences) which later allow removal (e.g., by means of cre recombinase) of a specific DNA sequence.

Elements necessary for Agrobacterium-mediated transformation may include, but not limited to, the right and/or, optionally, left border of the T-DNA or the vir region.

Multiple cloning sites (MCS) can be included in the recombinant construct or the vector of the invention to enable and facilitate the insertion of one or more nucleic acid sequences.

2.4 Vectors for Plant Transformation

If Agrobacteria are used for plant transformation, the recombinant construct is to be integrated into specific plasmid vectors, either into a shuttle or intermediate vector, or into a binary vector. If a Ti or Ri plasmid is to be used for the transformation, at least the right border, but in most cases the right and the left border, of the Ti or Ri plasmid T-DNA is flanking the region with the recombinant construct to be introduced into the plant genome. Preferably, binary vectors for the Agrobacterium transformation can be used. Binary vectors are capable of replicating both in E. coli and in Agrobacterium. They preferably comprise a selection marker gene and a linker or polylinker flanked by the right and, optionally, left T-DNA border sequence. They can be transformed directly into Agrobacterium (Holsters et al., Mol. Gen. Genet., 1978, 163:181-187). A selection marker gene may be included in the vector which permits a selection of transformed Agrobacteria (e.g., the nptIII gene). The Agrobacterium, which acts as host organism in this case, may already comprise a disarmed (i.e. non-oncogenic) plasmid with the vir region for transferring the T-DNA to the plant cell. The use of T-DNA for the transformation of plant cells has been studied and described extensively (e.g., EP 0120516; Hoekema, In: The Binary Plant Vector System, Offsetdrukkerij Kanters B. V., Alblasserdam, Chapter V; An et al., EMBO J., 1985, 4:277-287). A variety of binary vectors are known and available for transformation using Agrobacterium, such as, for example, pBI101.2 or pBIN19 (Clontech Laboratories, Inc. USA; Bevan et al., Nucl. Acids Res., 1984, 12:8711), pBinAR, pPZP200 or pPTV.

Transformation can also be realized without the use of Agrobacterium. Non-Agrobacterium transformation circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include, but not limited to, transformation via particle bombardment, protoplast uptake (e.g., PEG and electroporation) and microinjection, all are well known in the art. The choice of vector depends largely on the preferred selection for the species being transformed. Typical vectors suitable for non-Agrobacterium transformation include pCIB3064, pSOG19, and pSOG35 (see e.g., U.S. Pat. No. 5,639,949).

3. Introduction of Expression Cassette into Cells and Organisms

The aforementioned expression cassettes, or the recombinant constructs or vectors derived therefrom, can be introduced into a cell or an organism in various ways known to the skilled worker. “To introduce” is to be understood in the broad sense and comprises, for example, all those methods suitable for directly or indirectly introducing a DNA or RNA molecule into an organism or a cell, compartment, tissue, organ or seed of same, or generating it therein. The introduction can bring about either a transient presence or a stable presence of such a DNA or RNA molecule in the cell or organism.

Thus, a further aspect of the invention relates to cells and organisms (e.g., plants, plant cells, microorganisms, bacteria, etc.), which comprise at least one expression cassette of the invention, or a recombinant construct or a vector derived therefrom. In certain embodiments, the cell is suspended in culture, while in other embodiments the cell is in, or in part of, a whole organism, such as a microorganism or a plant. The cell can be prokaryotic or of eukaryotic nature. For plants or plant cells, preferably the expression cassette or recombinant construct is integrated into the genomic DNA, more preferably within the chromosomal or plastidic DNA, most preferably in the chromosomal DNA of the cell. For microorganisms, the expression cassette or recombinant construct is preferably incorporated into a plasmid or vector, which is then introduced into the microorganism. Accordingly, in one embodiment, the present invention relates to a transformed plant cell, plant or part thereof, comprising in its genome at least one stably incorporated expression cassette of the present invention, or a recombinant construct or a vector derived therefrom. In another embodiment, the present invention relates to a transformed microorganism comprising a plasmid or vector containing the expression cassette or recombinant construct of the present invention.

Preferred prokaryotic cells include mainly bacteria such as bacteria of the genus Escherichia, Corynebacterium, Bacillus, Clostridium, Proionibacterium, Butyrivibrio, Eubacterium, Lactobacillus, Erwinia, Agrobacterium, Flavobacterium, Alcaligenes, Phaeodacoilum, Colpidium, Mortierella, Entomophthora, Mucor, Crypthecodinium or Cyanobacteria, for example of the genus Synechocystis. Microorganisms which are preferred are mainly those which are capable of infecting plants and thus of transferring the expression cassette or construct of the invention. Preferred microorganisms are those of the genus Agrobacterium and in particular the species Agrobacterium tumefaciens and Agrobacterium rhizogenes.

Eukaryotic cells and organisms comprise plant and animal (preferably non-human) organisms and/or cells and eukaryotic microorganisms such as, for example, yeasts, algae or fungi. Preferred fungi include Aspergillus, Trichoderma, Ashbya, Neurospora, Fusarium, Beauveria or those described in Indian Chem. Engr., Section B., 1995, 37(1,2):15, Table 6. Especially preferred is the filamentous Hemiascomycete Ashbya gossypii. Preferred yeasts include Candida, Saccharomyces, Hansenula or Pichia, especially preferred are Saccharomyces cerevisiae or Pichia pastoris (ATCC Accession No. 201178). Preferred eukaryotic cells or organisms comprise plant cells and/or organisms, or eukaryotic microorganisms. A corresponding transgenic organism can be generated for example by introducing a desired expression system into a cell derived from such an organism by ways and methods known in the art.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The terms “seed” and “grain” are used interchangeably herein. A plant may be an inbred plant, an F1 hybrid, or any progeny of an F1 hybrid such as an F2, F3, F4, or F5 hybrid. The term “plant” may also include parts of plants, such as pollen, flowers, kernels, ears, cobs, leaves, husks, stalks, and the like. The term “plant” also encompasses plant cells, plant protoplasts, plant cell tissue cultures, callus tissue, embryos, meristematic regions, gametophytes, sporophytes, pollen and microspores, gamete producing cells, and a cell that regenerates into a whole plant, again wherein each of the aforementioned comprises the gene/nucleic acid of interest.

Plants that are particularly useful in the present invention include all plants which belong to the superfamily Viridiplantae, in particular monocotyledonous and dicotyledonous plants including fodder or forage legumes, ornamental plants, food crops, trees or algae selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apiuni graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Bela vulgaris, Brassica spp. (e.g. Brassica napus, Brassica rapa ssp. including canola, oilseed rape, turnip rape), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Cotylus spp., Crataegus spp., Crocus sativus, Cucurbita spp., Cucumis spp., Cynara spp., Daucus carota, Desmodium spp., Dimocaipus longan, Dioscorea spp., Diospyros spp., Echinochloa spp., Elaeis (e.g. Elaeis guineensis, Elaeis oleifera), Eleusine coracana, Erianthus sp., Eriobonya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypiuni hirsutum, Helianthus spp. (e.g. Helianthus annuus), Hemerocallis fulva, Hibiscus spp., Hordeum spp. (e.g. Hordeum vulgare), Ipomoea batatas, Jatropha curcas, Juglans spp., Lactuca sativa, Lathyrus spp., Lens culinaris, Lesquerella fendleri (Gray) Wats Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca saliva, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Sorghum halepense, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp. Cyclotella cryptica, Navicula saprophila, Synechococcus 7002 and Anabaena 7120, Chlorella protothecoides, Dunaliella salina, Chlorella spp, Dunaliella tertiolecta, Gracilaria, Sargassum, Pleurochrisis carterae, Laminaria 3840 hyperbore, Laminaria saccharina, Gracialliaria, Sargassum, Botryccoccus braunii, Arthospira platensis, amongst others. Especially preferred are rice, oilseed rape, canola, soybean, corn (maize), cotton, sugarcane, micro algae, alfalfa, sorghum, and wheat.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

Preferably, the organisms are plant organisms. Preferred plants are selected in particular from among crop plants. More preferred plants include, but not limited to, maize, soybean, barley, alfalfa, sunflower, flax, linseed, oilseed rape, canola, sesame, safflower (Carthamus tinctorius), olive tree, peanut, castor-oil plant, oil palm, cacao shrub, or various nut species such as, for example, walnut, coconut or almond, soybean, cotton, peanut, sorghum, tobacco, sugarbeet, sugarcane, rice, wheat, rye, turfgrass, millet, sugarcane, tomato, or potato.

It is noted that a plant need not be considered a “plant variety” simply because it contains stably within its genome a transgene, introduced into a cell of the plant or an ancestor thereof. In addition to a plant, the present invention provides any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated offspring, progeny, clone or descendant of such a plant, or any part or propagule of said plant, offspring, clone or descendant. Genetically modified plants according to the invention, which can be consumed by humans or animals, can also be used as food or feedstuffs, for example directly or following processing known in the art, or be used in biofuel production. The present invention also provides for parts of the organism especially plants, particularly reproductive or storage parts. Plant parts, without limitation, include seed, endosperm, ovule, pollen, roots, tubers, stems, leaves, stalks, fruit, berries, nuts, bark, pods, seeds and flowers.

The expression cassette of the invention, or a recombinant construct or vector derived therefrom, is typically introduced or administered in an amount that allows delivery of at least one copy per cell. Higher amounts (for example at least 5, 10, 100, 500 or 1000 copies per cell) can, if appropriate, result in a more efficient phenotype (e.g., higher expression or higher suppression of the target gene). The amount of the expression cassette, recombinant construct, or vector administered to a cell, tissue, or organism depends on the nature of the cell, tissue, or organism, the nature of the target gene, and the nature of the expression cassette, recombinant construct, or vector, and can readily be optimized to obtain the desired level of expression or inhibition.

Preferably at least about 100 molecules, preferably at least about 1000, more preferably at least about 10,000 of the expression cassette, recombinant construct, or vector, most preferably at least about 100,000 of the expression cassette, recombinant construct, or vector are introduced. In the case of administration of the expression cassette, recombinant construct, or vector to a cell culture or to cells in tissue, by methods other than injection, for example by soaking, electroporation, or lipid-mediated transfection, the cells are preferably exposed to similar levels of the expression cassette, recombinant construct, or vector in the medium.

For example, the expression cassette, recombinant construct, or vector of the invention may be introduced into cells via transformation, transfection, injection, projection, conjugation, endocytosis, and phagocytosis, all are well known in the art. Preferred methods for introduction include, but not limited to:

(a) methods of direct or physical introduction of the expression cassette, recombinant construct, or vector of the invention into the target cell or organism, and

(b) methods of indirect introduction of the expression cassette, recombinant construct, or vector of the invention into the target cell or organism by, for example, a first introduction of a recombinant construct and a subsequent intracellular expression.

### 4. Plant Transformation Techniques

In a further embodiment, the invention provides a method of producing a transgenic plant, plant cell, or plant part comprising:

(a) transforming a plant or plant cell with at least one aforementioned expression cassettes, or a recombinant construct or vector derived therefrom, and

(b) optionally regenerating from the plant cell a transgenic plant.

A variety of methods for introducing nucleic acid sequences (e.g., vectors) into the genome of plants and for the regeneration of plants from plant tissues or plant cells are known in the art (Plant Molecular Biology and Biotechnology, Chapter 6-7, pp. 71-119, CRC Press, Boca Raton, Fla., 1993; White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Annu. Rev. Plant Physiol. Plant Mol. Biol., 1991, 42:205-225; Halford et al., Br. Med. Bull., 2000, 56(1):62-73).

4.1 Non-Agrobacterium Transformation

Transformation methods may include direct and indirect methods of transformation. Suitable direct methods include, but not limited to, polyethylene glycol induced DNA uptake, liposome-mediated transformation (U.S. Pat. No. 4,536,475), biolistic methods using the gene gun (Fromm et al., Bio/Technology, 1990, 8(9):833-839; Gordon-Kamm et al., Plant Cell, 1990, 2:603), electroporation, incubation of dry embryos in DNA-comprising solution, and microinjection. In the case of these direct transformation methods, the plasmid used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

4.2 Agrobacterium Transformation

Transformation can also be carried out by bacterial infection by means of Agrobacterium (for example EP 0116718), viral infection by means of viral vectors (EP 0067553; U.S. Pat. No. 4,407,956; WO 95/34668; WO 93/03161) or by means of pollen (EP 0270356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch et al., Science, 1985, 227:1229-1231. The Agrobacterium-mediated transformation is best suited to dicotyledonous plants but has also been adopted to monocotyledonous plants. The transformation of plants by Agrobacteria is described in, for example, White F. F., “Vectors for Gene Transfer in Higher Plants,” in Transgenic Plants, Vol. 1, Engineering and Utilization, Kung and Wu, eds., Academic Press, pp. 15-38, 1993; Jenes et al., “Techniques for Gene Transfer,” in Transgenic Plants, Vol. 1, Engineering and Utilization, Kung and Wu, eds., Academic Press, pp. 128-143, 1993; Potrykus, Annu. Rev. Plant Physiol. Plant Mol. Biol., 1991, 42:205-225.

Transformation may result in transient or stable transformation and expression. Although an expression cassette of the present invention can be inserted into any plant and plant cell falling within these broad classes, it is particularly useful in crop plant cells.

Various tissues are suitable as starting material (explant) for the Agrobacterium-mediated transformation process including, but not limited to, callus (U.S. Pat. No. 5,591,616; EP 604662), immature embryos (EP 672752), pollen (U.S. Pat. No. 5,929,300), shoot apex (U.S. Pat. No. 5,164,310), or in planta transformation (U.S. Pat. No. 5,994,624). The method and material described herein can be combined with Agrobacterium mediated transformation methods known in the art.

4.3 Plastid Transformation

In another embodiment, the expression cassette or recombinant construct is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is extensively described in, for example, U.S. Pat. No. 5,451,513, U.S. Pat. No. 5,545,817, U.S. Pat. No. 5,545,818, U.S. Pat. No. 5,877,462, WO 95/16783, WO 97/32977, and in McBride et al., Proc. Natl. Acad. Sci. USA, 1994, 91:7301-7305. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., Proc. Natl. Acad. Sci. USA, 1990, 87:8526-8530; Staub et al., Plant Cell, 1992, 4:39-45). The presence of cloning sites between these markers allowed creation of a plastid targeting vector for introduction of foreign genes (Staub et al., EMBO J., 1993, 12:601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 1993, 90:913-917). Other selectable markers useful for plastid transformation are known in the art and encompassed within the scope of the invention.

### 5. Selection and Regeneration Techniques

To select cells which have successfully undergone transformation, it is preferred to introduce a selectable marker which confers, to the cells which have successfully undergone transformation, a resistance to a biocide (for example a herbicide), a metabolism inhibitor such as 2-deoxyglucose-6-phosphate (WO 98/45456) or an antibiotic. The selection marker permits the transformed cells to be selected from untransformed cells (McCormick et al., Plant Cell Reports, 1986, 5:81-84). Suitable selection markers are described above.

Transgenic plants can be regenerated in the known manner from the transformed cells. The resulting plantlets can be planted and grown in the customary manner. Preferably, two or more generations should be cultured to ensure that the genomic integration is stable and hereditary. Suitable methods are described in, for example, Fennell et al., Plant Cell Rep., 1992, 11:567-570; Stoeger et al., Plant Cell Rep., 1995, 14:273-278; and Jahne et al., Theor. Appl. Genet., 1994, 89:525-533.

### 6. Measurement of TPS and TPP Activity

### TPS Activity

Methods to determine enzymatic activity of TPS polypeptides are well known in the art. Typically the level of Tre6P (trehalose 6-phosphate), which is the product of the reaction catalyzed by TPS, is measured to infer the activity of the TPS enzyme. For example, Lunn et al. (2006, Biochem J. 397:139-148), describe a novel method using LC-MS-Q3 to measure the level of Tre6P in plants with 100 fold higher sensitivity. Blazquez et al. (1994) in FEMS Microbiol Lett. 121:223-227 describe a procedure for the quantitation of Tre6P based on its ability to inhibit hexokinase from Yarrowia lipolytica. Van Vaeck et al. (2001, Biochem J. 353:157-162) describe a method to determine Tre6P levels using a B. substilis phosphotrehalase enzymatic assay. In vivo activity of TPS polypeptides may also be determined, for example, through complementation assays in S. cerevisiae (Blazquez et al., 1998, Plant J. 13:685-689).

### TPP Activity

Methods to determine enzymatic activity of TPP polypeptides are well known in the art. Typically the levels of trehalose, which is the product of the reaction catalyzed by TPP, are measured. For example, a method using gas chromatography-mass spectrometry (GC-MS) analysis may be used such as the method described by Vogel et al. (1998, J. Exp. Bot. 52:1817-1826). Alternatively a method using trehalase may be used (Canovas et al., 2001, J. Bacteriol. 183:3365-337; Kienle et al. (1993, Yeast 9:607-611). Further alternative biochemical assays to determine TPP activity by measuring the amount of Pi released from Tre6P have been described (Kluuts et al., 2003, J. Biol. Chem. 278: 2093-2100). In vivo activity of TPP polypeptides may also be determined, for example, through complementation assays in S. cerevisiae (Shima et al., 2007, FEBS J. 274(5): 1192-1201; Vogel et al., 1998, Plant J 13:673-83).

### TPS-TPP Activity

The TPS and TPP activity of a TPS-TPP polypeptide may be determined using any of the methods described above. Specific methods to measure TPS and TPP activity adapted to test the effect of the physical proximity of the TPS and TPP enzymes which catalyze a sequential reaction have been previously described (Seo et al., 2000, Applied and Environmental Microbiology 66:2484-2490).

### 7. Biotechnological Applications

The expression cassettes, and recombinant constructs and vectors derived therefrom, can be used to manipulate the production of protein, oils, and/or amino acids and the like in a plant or plant cell. The invention, in one embodiment, provides a method for increasing one or more of protein, oil or one or more amino acids in a plant, plant cell, or plant part relative to a corresponding wild-type plant, plant cell, or plant part, comprising:

(a) obtaining a plant, plant cell, or plant part comprising at least one aforementioned expression cassette, or at least one recombinant construct or vector derived therefrom, and

(b) selecting a plant, plant cell, or plant part with an increase in one or more of protein, oil, or one or more amino acids.

Preferably, expression of the nucleic acid sequence comprised in the aforementioned expression cassettes in the transformed and/or regenerated transgenic plant increases the protein, oil, and/or amino acid content of the transgenic plant, plant cell, or part thereof, as compared to a corresponding wild-type plant, plant cell, or plant part. Methods of transforming a plant, plant cell, or plant part, selecting a transformed plant, plant cell, or plant part, and regenerating a plant from a plant, plant cell, or plant part are well known to one skilled in the art in view of the disclosure herein above.

Increases in protein, oil and amino acid content can be assessed by various methods known to one skilled in the art.

Plants suitable for the use in the methods of the invention can be monocotyledonous or dicotyledonous plants. In a preferred embodiment, the plant is a monocotyledonous plant, and more preferably, a maize plant, or the plant cell or plant part is from a monocotyledonous plant, preferably a maize plant.

The plant cell, plant, or plant part that is obtained from the aforementioned methods can be used for production of a food or feed composition or a food or feed supplement. Food or feed compositions include meal produced from the seed of a plant, such as corn meal or soybean meal. Food or feed compositions also include silage or forage. Accordingly, in a further embodiment, the present invention relates to the use of the plant cell, plant, or plant part obtained according to the aforementioned methods for the preparation of a food or feed composition or a composition intended for use as a food or feed supplement. The invention further relates to a method of producing a food or feed composition intended for animal or livestock feed comprising the plant, plant cell, or plant part obtained according to the aforementioned methods, and to the composition intended for animal or livestock feed thus obtained. In a preferred embodiment, said plant is a monocotyledonous plant, and more preferably, a maize plant.

In one embodiment, the plants, seed, or grain of the invention are used for production of human food, animal or livestock feed, as raw material in industry, pet foods, and food products. Such products can provide increased nutrition because of the increased nutrient value. In a further embodiment, the present invention also relates to animal feed which is formulated for a specific animal type, for example, as in U.S. Pat. No. 6,774,288, which is hereby incorporated by reference in its entirety. The seed or grain with increased protein, oil and/or amino acid content may be seed or grain from any crop species including a high protein maize, for example, as in U.S. Pat. No. 6,774,288, which is hereby incorporated by reference in its entirety. The animal feed may be used for feeding non ruminant animals, such as swine, poultry, horses, or sheep, small companion animals such as eats or dogs, and fish such as tilapia or salmon. For example, maize is used extensively as livestock feed, primarily for beef cattle, dairy cattle, hogs, and poultry. See, for example, Chang et al. in U.S. Pat. Nos. 7,087,261 and 6,774,288 and in U.S. Publ. No. 2005/0246791.

### 8. Plant Breeding

8.1 Traditional Breeding Methods

The plants and plant parts obtained from the aforementioned methods can also be used in a plant breeding program. In one embodiment, this invention relates to methods for producing a maize plant by crossing a first parent maize plant with a second parent maize plant wherein either the first or second parent maize plant comprises an expression cassette or recombinant construct described herein. The other parent may be any other maize plant, such as another inbred line or a plant that is part of a cultivated or natural population. Any plant breeding method may be used, including but not limited to selling, ribbing, backcrossing, recurrent selection, mass selection, pedigree breeding, double haploids, bulk selection, hybrid production, crosses to populations, and the like. These methods are well known in the art.

For example, pedigree breeding is used commonly for the improvement of self-pollinating crops or inbred lines of cross-pollinating crops. Pedigree breeding starts with the crossing of two genotypes, such as a first inbred line comprising an expression cassette or recombinant construct described herein and a second elite inbred line having one or more desirable characteristics that is lacking or which complements the first inbred line. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Several different physiological and morphological characteristics can be selected for as attributes of the recurrent parent in a backcross breeding program, including days to maturity (e.g. days from emergence to 50% of plants in silk or 50% of plants in pollen), plant height, ear height, average length of top ear internode, average number of tillers, average number of ears per stalk, anthocyanin content of brace roots, width of ear node leaf, length of ear node leaf, number of leaves above top ear, leaf angle from second leaf above ear at anthesis to stalk above leaf, leaf color, leaf sheath pubescence, leaf marginal waves, leaf longitudinal creases, number of lateral branches on tassel, branch angle from central spike of tassel, tassel length, pollen shed, anther color, glume color, bar glumes, ear silk color, fresh husk color, dry husk color, position of ear, husk tightness, husk extension, ear length, ear diameter at mid-point, ear weight, number of kernel rows, kernel rows, row alignment, shank length, ear taper, kernel length, kernel width, kernel thickness, kernel shape, aleurone color pattern, aleurone color, hard endosperm color, endosperm type, weight per 100 kernels, cob diameter at mid-point, cob color, and agronomic traits such as stay green (late season plant health), dropped ears (percentage of plants that dropped an ear prior to harvest), pre-anthesis brittle snapping (stalk breaking near the time of pollination), pre-anthesis root lodging (lean from the vertical axis at an approximate 30° angle or greater near the time of pollination), and post-anthesis root lodging.

8.2 Breeding with Molecular Markers

Molecular markers, which includes markers identified through the use of techniques such as Isozyme Electrophoresis, Restriction Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods utilizing the inbred of the present invention. Molecular markers can be used to identify the unique genetic composition of the invention and progeny lines retaining that unique genetic composition. Various molecular marker techniques may be used in combination to enhance overall resolution.

One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and can minimize the amount of genome from the donor parent that remains in the selected plants. It can also be used to reduce the number of crosses back to the recurrent parent needed in a backcrossing program. The use of molecular markers in the selection process is often called genetic marker enhanced selection.

Descriptions of breeding methods can also be found in one of several reference books (e.g., Allard, Principles of Plant Breeding, 1960; Simmonds, Principles of Crop Improvement, 1979; Fehr, “Breeding Methods for Cultivar Development”, Production and Uses, 2nd ed., Wilcox editor, 1987). See also U.S. Pat. No. 7,183,470 and U.S. Pat. No. 7,339,097, the disclosures of which are expressly incorporated herein by reference.

8.3 Maize Hybrids

A single cross maize hybrid results from the cross of two inbred lines, each of which has a genotype that complements the genotype of the other. The hybrid progeny of the first generation is designated F1. In the development of commercial hybrids in a maize plant breeding program, only the F1 hybrid plants are sought. F1 hybrids are more vigorous than their inbred parents. This hybrid vigor, or heterosis, can be manifested in many polygenic traits, including increased vegetative growth and increased yield.

An inbred maize line comprising an expression cassette or recombinant construct described herein may be used to produce hybrid maize. One such embodiment is the method of crossing the inbred maize line comprising an expression cassette or recombinant construct of the invention with another maize plant, such as a different maize inbred line, to form a first generation F1 hybrid seed. The first generation F1 hybrid seed, plant and plant part produced by this method is an embodiment of the invention. The first generation F1 seed, plant and plant part will comprise an essentially complete set of the alleles of the inbred line comprising an expression cassette or recombinant construct described herein. One of ordinary skill in the art can utilize either breeder books or molecular methods to identify a particular F1 hybrid plant produced using the inbred line comprising an expression cassette or recombinant construct described herein. Further, one of ordinary skill in the art may also produce F1 hybrids with transgenic, male sterile and/or backcross conversions of the inbred line comprising an expression cassette or recombinant construct described herein.

The development of a maize hybrid in a maize plant breeding program involves three steps: (1) the selection of plants from various germplasm pools for initial breeding crosses; (2) the selfing of the selected plants from the breeding crosses for several generations to produce a series of inbred lines, such as an inbred line comprising an expression cassette or recombinant construct described herein, which, although different from each other, breed true and are highly uniform; and (3) crossing the selected inbred lines with different inbred lines to produce the hybrids. During the inbreeding process in maize, the vigor of the lines decreases, and so one would not be likely to use an inbred line comprising an expression cassette or recombinant construct described herein directly to produce grain. However, vigor can be restored by crossing the inbred line comprising an expression cassette or recombinant construct described herein with a different inbred line to produce a commercial F1 hybrid. An important consequence of the homozygosity and homogeneity of the inbred line is that the hybrid between a defined pair of inbreds may be reproduced indefinitely as long as the homogeneity of the inbred parents is maintained.

The inbred line comprising an expression cassette or recombinant construct described herein may be used to produce a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C.

One or more genetic traits which have been engineered into the genome of a particular maize plant or plants using transformation techniques could be moved into the genome of another line using traditional breeding techniques that are well known in the plant breeding arts. For example, a backcrossing approach is commonly used to move a transgene from a transformed maize plant to an elite inbred line, and the resulting progeny would then comprise the transgene(s). In a single gene converted plant, the plant would have essentially all the desired morphological and physiological characteristics of the inbred in addition to the single gene transferred via backcrossing or via genetic engineering. Also, if an inbred line was used for the transformation then the transgenic plants could be crossed to a different inbred in order to produce a transgenic hybrid maize plant. In the same manner, more than one transgene can be transferred into the inbred.

Hybrid plants produced by the plant breeding methods described above may be used for producing grain with an increase in protein, oil, and/or one or more amino acids by interplanting at least two hybrid plant populations. For example, hybrid seed comprising an expression cassette or recombinant construct described herein may be interplanted with another hybrid seed with high yield to obtain grain with increased protein, oil, and/or one or more amino acids at competitive yields. The invention includes methods for producing grain by planting a first hybrid seed comprising an expression cassette or recombinant construct described herein, and at least a second hybrid seed; growing the seed under conditions that result in cross pollination between the plant produced from the seed of the first hybrid and the plant produced by the seed of the second hybrid; and harvesting the grain. Conditions that result in cross pollination between the hybrid plants include interplanting the hybrid populations in close enough proximity to allow for pollen transfer between the hybrid populations, and timing the planting of the hybrids such that pollen is released from one of the hybrids when the other hybrid is receptive to pollination. Methods of producing grain with increased value through interplanting of two or more hybrids are described, for example, in WO2010/025213.

### Sequence Descriptions:

| | Nucleo- | Amino |
|---|---|---|
| | tide SEQ | Acid SEQ |
| Sequence | ID NO | ID NO |
| AtTPS8 | 1 | 2 |
| AtTPS9 | 3 | 4 |
| SpFdx DNA sequence, unmodified, variant 1 | 5 | 6 |
| ZmGlb1 promoter (embryo-specific) | 7 | — |
| ScBV254 promoter (constitutive, shorter version) | 8 | — |
| ScBV promoter (constitutive, longer version) | 9 | — |
| Met1-1 intron | 10 | — |
| NOS terminator | 11 | — |
| MADS3 intron | 12 | — |
| OCS3 terminator | 13 | — |
| KG86_12a promoter (whole-seed specific) | 14 | — |
| 27 kDa zein promoter (endosperm specific) | 15 | — |
| AtTPS5, TPS homolog from A. thaliana | 16 | 17 |
| AtTPS11, TPS homolog from A. thaliana | 18 | 19 |
| GmTPS-like, TPS homolog from G. max | 20 | 21 |
| OsTPS700, TPS homolog from O. sativa | 22 | 23 |
| OsTPS300, TPS homolog from O. sativa | 24 | 25 |
| OsTPS360, TPS homolog from O. sativa | 26 | 27 |
| StTPS-like, TPS homolog from S. tuberosum | 28 | 29 |
| CwTPS-like, TPS homolog from C. watsonii | 30 | 31 |
| YliTPS-like, TPS homolog from Y. lypolitica | 32 | 33 |
| TPS homolog from A. lyrata subsp. lyrata | 34 | 35 |
| TPS homolog from A. lyrata subsp. lyrata | 36 | 37 |
| Sequence motif from Pfam PF00982.15 | — | 38 |
| Sequence motif from Pfam PF00982.15 | — | 39 |
| Sequence motif from Pfam PF00982.15 | — | 40 |
| Sequence motif from Pfam PF00982.15 | — | 41 |
| Sequence motif from Pfam PF00982.15 | — | 42 |
| Sequence motif from Pfam PF02358.10 | — | 43 |
| Sequence motif from Pfam PF02358.10 | — | 44 |
| Sequence motif from Pfam PF02358.10 | — | 45 |
| Sequence motif from Pfam PF02358.10 | — | 46 |
| Sequence motif from Pfam PF02358.10 | — | 47 |
| Sequence motif from Pfam PF02358.10 | — | 48 |
| Sequence motif from Pfam PF02358.10 | — | 49 |
| AtTPS8.Zm, codon optimized for Z. mays | 50 | 2 |
| AtTPS9.Zm, codon optimized for Z. mays | 51 | 4 |
| Maize ubiquitin intron | 52 | — |
| TPS homolog from A. thaliana | 53 | 54 |
| TPS homolog from S. bicolor | 55 | 56 |
| TPS homolog from S. lycopersicum | 57 | 58 |
| TPS homolog from T. aestivum | 59 | 60 |
| TPS homolog from Z. marina | 61 | 62 |
| TPS homolog from Z. mays | 63 | 64 |
| TPS homolog from Z. mays | 65 | 66 |
| TPS homolog from Z. mays | 67 | 68 |
| MAWS42 promoter (whole-seed specific) | 69 | — |
| Ubiquitin promoter from Z. mays (constitutive) | 70 | — |
| 10 kDa Zein promoter (endosperm specific) | 71 | — |
| Consensus sequence for FIG. 1 | — | 72 |
| Modified transit peptide SpFdx | 73 | 6 |
| Atc17 intron | 74 | — |
| Atss1 intron | 75 | — |
| TOI3357 terminator | 76 | — |
| KG86 promoter (whole-seed specific) | 77 | |

The following examples serve to illustrate certain embodiments and aspects of the present invention and are not to be construed as limiting the scope thereof.

### EXAMPLES

### Example 1

### Construction of Expression Cassettes

General cloning processes such as, for example, restriction digests, agarose gel electrophoresis, purification of DNA fragments, PCR amplification, transformation of E. coli cells, growth of bacteria and sequence analysis of recombinant DNA were carried out as described in Sambrook and Russell. (2001, Molecular Cloning: A Laboratory Manual, Third Edition, Cold Spring Harbor Laboratory Press: ISBN 0-87969-577-3), Kaiser et al. (1994, “Methods in Yeast Genetics,” Cold Spring Harbor Laboratory Press: ISBN 0-87969-451-3), or “Gateway® Technology,” Version E, (Invitrogen, (Carlsbad, Calif.), 2010, see webpage at tools.invitrogen.com/content/sfs/manuals/gatewayman.pdf). Specific cloning methods include ligation of DNA fragments, ligation independent cloning (LIC), and/or Gateway cloning as described in Sambrook and Russell. (2001, Molecular Cloning: A Laboratory Manual, Third Edition, Cold Spring Harbor Laboratory Press: ISBN 0-87969-577-3), or “Gateway® Technology,” Version E, (Invitrogen, (Carlsbad, Calif.), 2010, see webpage at tools.invitrogen.com/content/sfs/manuals/gatewayman.pdf).

AtTPS8 and AtTPS9 are Arabidopsis Class II trehalose-6-phosphate synthases that contain the PF00982.15 and PF02358.10 Pfam domains. AtTPS8 and AtTPS9 also contain the amino acid sequence motifs of SEQ ID NO: 38, 39, 40, 41, 42, 43, 44, 45, 46, 47, 48 and 49. Examples of expression cassettes for overexpression of AtTPS8 or AtTPS9 are shown in Table 4 below. For Constructs 1-4, the nucleic acid sequences encoding AtTPS8 and AtTPS9 were amplified by PCR. For Construct 5, the nucleic acid sequence encoding AtTPS9 was generated through reverse translation of the protein sequence, codon optimization of the resulting nucleotide sequence for expression in maize, and DNA synthesis. DNA synthesis is performed by a range of commercial vendors including Epoch Life Science (Missouri City, Tex.), Invitrogen, (Carlsbad, Calif.), Blue Heron Biotechnology (Bothell, Wash.) and DNA 2.0 (Menlo Park, Calif.). After PCR amplification or DNA synthesis, the nucleic acid sequences encoding AtTPS8 or AtTPS9 are cloned into standard cloning vectors and sequenced.

The expression cassettes were assembled in a cloning vector by cloning the DNA encoding AtTPS8 or AtTPS9 downstream of the ScBV, ScBV254, or ZmGlb1 promoter, and upstream of the NOS terminator region. The expression cassettes also contain the first intron of the rice metallothionein gene (Met 1-1) between the promoter and the coding region. In addition, Construct 3 contains the Fdx transit peptide between the Met1-1 intron and the AtTPS8 coding region. Constructs 23 and 24 contain the DNA encoding AtTPS5 downstream of the ScBV254 or KG86_{—}12a promoter, the first intron of the rice metallothionein gene (Met1-1), and upstream of the NOS terminator region. Maize plants containing Construct 1, 2, 3, 4, 17, 23, 24, or 25 were evaluated in field trials for yield and protein, oil, and amino acid content (see Examples 4 and 5).

TABLE 4
Examples of expression cassettes for overexpression
of AtTPS8 or AtTPS9.

| Construct | Cassette component | SEQ ID NOs |
|---|---|---|
| 1 | p-ScBV::i-Met1-1::AtTPS9::t-NOS | 9, 10, 3, 11 |
| 2 | p-ZmGlb1::i-Met1-1::AtTPS9::t-NOS | 7, 10, 3, 11 |
| 3 | p-ScBV::i-Met1-1::Fdx::AtTPS9::t-NOS | 9, 10, 5, 3, 11 |
| 4 | p-ZmGlb1::i-Met1-1::AtTPS8::t-NOS | 7, 10, 1, 11 |
| 5 | p-ScBV254::i-Met1::AtTPS9.Zm::t-NOS | 8, 10, 51, 11 |
| 6 | p-ScBV254::i-Met1-1::GmTPS::t-NOS | 8, 10, 20, 11 |
| 7 | p-KG86_12a::i-Met1-1::GmTPS::t-NOS | 14, 10, 20, 11 |
| 8 | p-ScBV254::i-Met1-1::OsTPS360::t-NOS | 8, 10, 26, 11 |
| 9 | p-KG86_12a::i-Met1-1::OsTPS360::t-NOS | 14, 10, 26, 11 |
| 10 | p-ScBV254::i-Met1-1::StTPS::t-NOS | 8, 10, 28, 11 |
| 11 | p-KG86_12a::i-Met1-1::StTPS::t-NOS | 14, 10, 28, 11 |
| 12 | p-ScBV254::i-Met1-1::YliTPS::t-NOS | 8, 10, 32, 11 |
| 13 | p-ScBV254::1-Met1-1::CwTPS::t-NOS | 8, 10, 30, 11 |
| 14 | p-ScBV254::i-Met1-1::AtTPS11::t-NOS | 8, 10, 18, 11 |
| 15 | p-ScBV254::i-Met1::AtTPS9::t-NOS | 8, 10, 3, 11 |
| 16 | p-ScBV254::i-Met1::AtTPS9::t-OCS3 | 8, 10, 3, 13 |
| 17 | p-KG86_12a::i-Met1-1::AtTPS9::t-NOS | 14, 10, 3, 11 |
| 18 | p-10kDaZein::AtTPS9::t-OCS3 | 71, 3, 13 |
| 19 | p-UBI::i-Ubi::AtTPS9::t-OCS3 | 70, 52, 3, 13 |
| 20 | p-10kDaZein::i-Met1-1::AtTPS9.Zm::t-NOS | 71, 10, 51, 11 |
| 21 | p-27kDaZein::1-MADS3::AtTPS8.Zm::t-OCS3 | 15, 12, 50, 13 |
| 22 | p-ScBV254::i-Atc17::AtTPS9.Zm::t-TOI3357 | 8, 74, 51, 76 |
| 23 | p-KG86_12a::i-Met1-1::AtTPS5::t-NOS | 14, 10, 16, 11 |
| 24 | p-ScBV254::i-Met1-1::AtTPS5::t-NOS | 8, 10, 16, 11 |
| 25 | p-KG86::i-Met1-1::AtTPS9::t-NOS | 77, 10, 3, 11 |

Examples of additional expression cassettes for overexpression of TPS homologs are assembled by the methods described above. Each of these expression cassettes contains a nucleic acid molecule encoding a TPS homolog and the additional cassette component(s) described in Table 5 below. “-” indicates that the expression cassette does not contain the listed component.

TABLE 5
Examples of additional expression cassettes
for overexpression of TPS homologs.

| Promoter | Intron | Transit peptide | Terminator |
|---|---|---|---|
| SEQ ID NO | SEQ ID NO | SEQ ID NO | SEQ ID NO |
| 7 | 10 | 5 or 73 | 11 |
| 7 | 10 | 5 or 73 | — |
| 7 | 10 | — | 11 |
| 7 | 10 | — | — |
| 7 | — | 5 or 73 | 11 |
| 7 | — | 5 or 73 | — |
| 7 | — | — | 11 |
| 7 | — | — | — |
| 8 | 10 | 5 or 73 | 11 |
| 8 | 10 | 5 or 73 | — |
| 8 | 10 | — | 11 |
| 8 | 10 | — | — |
| 8 | — | 5 or 73 | 11 |
| 8 | — | 5 or 73 | — |
| 8 | — | — | 11 |
| 8 | — | — | — |
| 9 | 10 | 5 or 73 | 11 |
| 9 | 10 | 5 or 73 | — |
| 9 | 10 | — | 11 |
| 9 | 10 | — | — |
| 9 | — | 5 or 73 | 11 |
| 9 | — | 5 or 73 | — |
| 9 | — | — | 11 |
| 9 | — | — | — |

### Example 2

### Construction of Plant Transformation Vectors

Plant transformation binary vectors such as pBi-nAR are used (Höfgen & Willmitzer 1990, Plant Sci. 66:221-230). Construction of the binary vectors was performed by ligation of the expression cassette into the binary vector. Further examples for plant binary vectors are the pSUN300 or pSUN2-GW vectors and the pPZP vectors (Hajdukiewicz et al., Plant Molecular Biology 25: 989-994, 1994). These binary vectors contain an antibiotic resistance gene under the control of the NOS promoter. Expression cassettes are cloned into the multiple cloning site of the pEntry vector using standard cloning procedures. pEntry vectors are combined with a pSUN destination vector to form a binary vector by the use of the GATEWAY technology (Invitrogen, webpage at invitrogen.com) following the manufacturer's instructions. The recombinant vector containing the expression cassette was transformed into Top10 cells (Invitrogen) using standard conditions. Transformed cells were selected on LB agar containing 50 μg/ml kanamycin grown overnight at 37° C. Plasmid DNA was extracted using the QIAprep Spin Miniprep Kit (Qiagen) following manufacturer's instructions. Analysis of subsequent clones and restriction mapping was performed according to standard molecular biology techniques (Sambrook et al., 1989, Molecular Cloning, A Laboratory Manual. 2nd Edition. Cold Spring Harbor Laboratory Press. Cold Spring Harbor, N.Y.).

### Example 3

### Plant Transformation

### Maize

Agrobacterium cells harboring a plasmid containing the gene of interest and the mutated maize AHAS gene were grown in YP medium supplemented with appropriate antibiotics for 1-2 days. One loop of Agrobacterium cells was collected and suspended in 1.8 ml M-LS-002 medium (LS-inf). The cultures were incubated while shaking at 1,200 rpm for 5 min-3 hrs. Corn cobs were harvested at 8-11 days after pollination. The cobs were sterilized in 20% Clorox solution for 5 min, followed by spraying with 70% Ethanol and then thoroughly rinsed with sterile water. Immature embryos 0.8-2.0 mm in size were dissected into the tube containing Agrobacterium cells in LS-inf solution.

The constructs were transformed into immature embryos by a protocol modified from Japan Tobacco Agrobacterium mediated plant transformation method (U.S. Pat. Nos. 5,591,616; 5,731,179; 6,653,529; and U.S. Patent Application Publication No. 2009/0249514). Two types of plasmid vectors were used for transformation. One type had only one T-DNA border on each of left and right side of the border, and selectable marker gene and gene of interest were between the left and right T-DNA borders. The other type was so called “two T-DNA constructs” as described in Japan Tobacco U.S. Pat. No. 5,731,179. In the two DNA constructs, the selectable marker gene was located between one set of T-DNA borders and the gene of interest was included in between the second set of T-DNA borders. Either plasmid vector can be used. The plasmid vector was electroporated into Agrobacterium.

Agrobacterium infection of the embryos was carried out by inverting the tube several times. The mixture was poured onto a filter paper disk on the surface of a plate containing co-cultivation medium (M-LS-011). The liquid agro-solution was removed and the embryos were checked under a microscope and placed scutellum side up. Embryos were cultured in the dark at 22° C. for 2-4 days, and transferred to M-MS-101 medium without selection and incubated for four to seven days. Embryos were then transferred to M-LS-202 medium containing 0.75 μM imazethapyr and grown for three weeks at 27° C. to select for transformed callus cells.

Plant regeneration was initiated by transferring resistant calli to M-LS-504 medium supplemented with 0.75 μM imazethapyr and growing under light at 26° C. for two to three weeks. Regenerated shoots were then transferred to a rooting box with M-MS-618 medium (0.5 μM imazethapyr). Plantlets with roots were transferred to soil-less potting mixture and grown in a growth chamber for a week, then transplanted to larger pots and maintained in a greenhouse until maturity.

Transgenic maize plant production is also described, for example, in U.S. Pat. Nos. 5,591,616 and 6,653,529; U.S. Patent Application Publication No. 2009/0249514; and WO/2006136596, each of which are hereby incorporated by reference in their entirety. Transformation of maize may be made using Agrobacterium transformation, as described in U.S. Pat. Nos. 5,591,616; 5,731,179; U.S. Patent Application Publication No. 2002/0104132 and the like. Transformation of maize (Zea mays L.) can also be performed with a modification of the method described by Ishida et al. (Nature Biotech., 1996, 14:745-750). The inbred line A188 (University of Minnesota) or hybrids with A188 as a parent are good sources of donor material for transformation (Fromm et al., Biotech, 1990, 8:833), but other genotypes can be used successfully as well. Ears are harvested from corn plants at approximately 11 days after pollination (DAP) when the length of immature embryos is about 1 to 1.2 mm Immature embryos are co-cultivated with Agrobacterium tumefaciens that carry “super binary” vectors and transgenic plants are recovered through organogenesis. The super binary vector system is described in WO 94/00977 and WO 95/06722. Vectors are constructed as described. Various selection marker genes are used including the maize gene encoding a mutated acetohydroxy acid synthase (AHAS) enzyme (U.S. Pat. No. 6,025,541). Similarly, various promoters are used to regulate the trait gene to provide constitutive, developmental, inducible, tissue or environmental regulation of gene transcription.

Excised embryos can be used and can be grown on callus induction medium, then maize regeneration medium, containing imidazolinone as a selection agent. The petri dishes are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes. Presence of the transgene and copy number was determined by TaqMan PCR.

### Wheat

A specific example of wheat transformation can be found in PCT Application No. WO 93/07256. Transformation of wheat can also be performed with the method described by Ishida et al. (Nature Biotech., 1996, 14:745-750). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system is described in WO 94/00977 and WO 95/06722, which are hereby incorporated by reference in its entirety. Vectors are constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, inducible, developmental, tissue or environmental regulation of gene transcription.

After incubation with Agrobacterium, the embryos are grown on callus induction medium, then regeneration medium, containing imidazolinone as a selection agent. The petri dishes are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

### Rice

Rice may be transformed using methods disclosed in U.S. Pat. Nos. 4,666,844; 5,350,688; 6,153,813; 6,333,449; 6,288,312; 6,365,807; 6,329,571, and the like.

### Soybean

Transformation of soybean can be performed using, for example, a technique described in European Patent No. EP 0424 047, U.S. Pat. No. 5,322,783, European Patent No. EP 0397 687, U.S. Pat. No. 5,376,543 or U.S. Pat. No. 5,169,770, or by any of a number of other transformation procedures known in the art. Soybean seeds are surface sterilized with 70% ethanol for 4 minutes at room temperature with continuous shaking, followed by 20% (v/v) bleach supplemented with 0.05% (v/v) TWEEN for 20 minutes with continuous shaking. Then the seeds are rinsed 4 times with distilled water and placed on moistened sterile filter paper in a petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile petri dish and air-dried to a moisture content less than 20% (fresh weight) in a sealed petri dish until further use.

Brassica napus

Canola may be transformed, for example, using methods such as those disclosed in U.S. Pat. Nos. 5,188,958; 5,463,174; 5,750,871; EP1566443; WO02/00900; and the like.

For example, seeds of canola are surface sterilized with 70% ethanol for 4 minutes at room temperature with continuous shaking, followed by 20% (v/v) CLOROX supplemented with 0.05% (v/v) TWEEN for 20 minutes, at room temperature with continuous shaking. Then, the seeds are rinsed four times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use.

Agrobacterium tumefaciens culture is prepared from a single colony in LB solid medium plus appropriate antibiotics (e.g. 100 mg/l streptomycin, 50 tang/1 kanamycin) followed by growth of the single colony in liquid LB medium to an optical density at 600 nm of 0.8. Then, the bacteria culture is pelleted at 7000 rpm for 7 minutes at room temperature, and resuspended in MS (Murashige et al., 1962, Physiol. Plant. 15:473-497) medium supplemented with 100 mM acetosyringone. Bacteria cultures are incubated in this pre-induction medium for 2 hours at room temperature before use. The axis of canola zygotic seed embryos at approximately 44% moisture content are imbibed for 2 hours at room temperature with the pre-induced Agrobacterium suspension culture. (The imbibition of dry embryos with a culture of Agrobacterium is also applicable to maize and soybean embryo axes). The embryos are removed from the imbibition culture and are transferred to petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/l carbenicillin or 300 mg/l cefotaxime to kill the Agrobacteria. The liquid medium is used to moisten the sterile filter paper. The embryos are incubated during 4 weeks at 25° C., under 440 mmol m²s¹ and a 12 hour photoperiod. Once the seedlings have produced roots, they are transferred to sterile soil. The medium of the in vitro plants is washed off before transferring the plants to soil. The plants are kept under a plastic cover for 1 week to favor the acclimatization process. Then the plants are transferred to a growth room where they are incubated at 25° C., under 440 mmol m²s¹ light intensity and 12-hour photoperiod for about 80 days.

Samples of the primary transgenic plants (T0) are analyzed by PCR to confirm the presence of T-DNA. These results can be confirmed by Southern hybridization wherein DNA is electrophoresed on a 1% agarose gel and transferred to a positively charged nylon membrane (Roche Diagnostics). The PCR DIG Probe Synthesis Kit (Roche Diagnostics) is used to prepare a digoxigenin labeled probe by PCR as recommended by the manufacturer.

### Example 4

### Yield and Grain Composition of F1 Hybrid Maize Plants

Transgenic events were produced by transformation of a maize inbred line with Construct 1, 2, 3, or 4. Homozygous events were planted in an isolated crossing block, detasseled, and open pollinated with a male tester to produce hybrid seed (F1 generation). The hybrid seed was used in field trials to evaluate grain yield and composition and were planted in three to twelve locations with two to four replications per location. Separate field trials were conducted for yield and analysis of grain composition. Field trials for yield were allowed to open pollinate. Field trials for composition were hand pollinated. However, either pollination method may be used for yield and composition trials. Trials were planted in a randomized complete block design, with all events per construct and corresponding isogenic non-transgenic hybrid controls. Data were collected from the composition trials for grain protein, oil and six amino acids (arginine, cysteine, lysine, methionine, threonine, and valine) on a percent dry weight basis. Data were generated for one to four hybrid combinations over one or two years. Data was subjected to ANOVA by using JMP, where locations were treated as blocks and means were separated at the 0.05 level of significance.

Transgenic events were also produced by transformation of a maize inbred line with Construct 17, 23, 24, or 25. Field trials for constructs 17, 23, 24, and 25 were based on an initial field screen with minimal replications and locations.

### Example 5

### Analysis of Protein, Oil, and Amino Acid Content

Protein content and content of one or more amino acids of transgenic and corresponding wild-type plants and seeds can be evaluated by methods known in the art, for example, as described for corn in U.S. Publication Serial No. 2005/0241020 which is hereby incorporated by reference in its entirety.

Protein and oil content was determined on a dry matter basis. Protein and oil content was measured by near-infrared (NIR) spectroscopy using a Perten DA7200 NIR analyzer and Partial Least Squares (PLS) calibration models developed based on nitrogen combustion and supercritical fluid extraction reference methods for measurement of total protein and total oil, respectively (Williams, P.; Norris, K., Eds. Near-Infrared Technology in the Agricultural and Food Industries, 2nd ed.; American Association of Cereal Chemists, Inc.: St. Paul, Minn., 2001; AACC, Approved Methods, 10th ed., AACC Method 39-00, Near-Infrared Methods—Guidelines for Model Development and Maintenance; American Association of Cereal Chemists, Inc.: St. Paul, Minn., 2000). Samples may also be analyzed for crude protein (2000, Combustion Analysis (LECO) AOAC Official Method 990.03), crude fat (2000, Ether Extraction, AOAC Official Method 920.39 (A)), and moisture (2000, vacuum oven, AOAC Official Method 934.01).

An example of amino acid analysis of transgenic seed can be found for corn in US 2005/0241020. For example, mature seed samples were ground with an IKA A11 basic analytical mill. Samples were analyzed for amino acids using a modified Association of Official Analytical Chemists (AOAC) official method (982.30 E (a, b, c), CHP 45.3.05, 2000), with four repetitions, modified by using the Waters AccuTag system on the Acquity HPLC platform. Samples may also be analyzed for complete amino acid profile (AAP) using the Association of Official Analytical Chemists (AOAC) official method (982.30 E (a, b, c), CHP 45.3.05, 2000).

Protein, oil, and amino acid content will vary widely from one location to another due to environmental effects such as weather conditions, nutrient availability, and soil moisture, as well as variation in agronomic conditions such as planting density. Thus, it is important to consider the relative difference between the transgenic hybrid and the isogenic hybrid control at each location to determine transgene effects.

Results of the field trials indicated that overexpression of AtTPS8 or AtTPS9 significantly increased protein, oil, and/or amino acid content in maize kernels. Constitutive expression of AtTPS9 via the ScBV promoter with no additional targeting significantly increased protein, oil and the amino acids arginine, cysteine, lysine, methionine, threonine and valine in two events with no significant decrease in yield (Construct 1, Tables 6 and 7). Constitutive expression of AtTPS9 combined with additional targeting to the plastid resulted in increased protein and oil content in two events with no significant decrease in yield (Construct 3, Tables 12 and 13). Embryo-specific expression of TPS via the ZmGlb1 promoter resulted mainly in significant increases in oil in several events with no significant decrease in yield (Construct 2, Tables 10 and 11). Embryo-specific expression of AtTPS8 via the ZmGlb1 promoter with no additional targeting significantly increased oil content (Construct 4, Tables 14 and 15). In initial field screens with minimal replications and locations, whole seed expression of AtTPS9 via the KG86_{—}12a promoter significantly increased protein and isoleucine content (Construct 17, data not shown). In initial field screens with minimal replications and locations, whole seed expression of AtTPS9 via the KG86 promoter showed similar trends as with the KG86_{—}12a promoter (Construct 25, data not shown). Constitutive expression via the ScBV254 promoter or whole seed expression via the KG86_{—}12a promoter of AtTPS5 did not show statistically significant increases in protein, oil, or the amino acids arginine, cysteine, lysine, methionine, threonine, and valine in the initial field screens which had minimal replications and locations (Constructs 23-24; data not shown).

TABLE 6
Summary of field data for Construct 1. Numbers shown in bold are significantly different from the control
at the p-value shown. bu/a is bushels per acre. “All” indicates analysis across all events. (T/C)%
is the value for the transgenic hybrid combination (T) expressed as a percent of the control (C).

| | | Yield | | | | | | | | |
|---|---|---|---|---|---|---|---|---|---|---|
| | | (bu/a) | Oil (%) | Protein (%) | Arg (%) | Cys (%) | Lys (%) | Met (%) | Thr (%) | Val (%) |
| Event | Desc. | p < 0.1 | p < 0.05 | p < 0.05 | p < 0.05 | p < 0.05 | p < 0.05 | p < 0.05 | p < 0.05 | p < 0.05 |
| All | (T/C)% | 105 | 108 | 110 | 110 | 114 | 106 | 110 | 108 | 108 |
| 1A | (T/C)% | 101 | 107 | 111 | 109 | 111 | 106 | 110 | 108 | 108 |
| 1B | (T/C)% | 108 | 112 | 108 | 108 | 112 | 104 | 110 | 106 | 105 |
| 1C | (T/C)% | 102 | 109 | 112 | 114 | 121 | 105 | 116 | 111 | 111 |
| 1D | (T/C)% | 103 | 105 | 109 | 108 | 109 | 107 | 105 | 107 | 107 |

TABLE 7
Field data for Construct 1. Numbers shown in bold are significantly different from the control at the p-value
shown. bu/a is bushels per acre. “All” indicates analysis across all events. (T/C)% is the value for
the transgenic hybrid combination (T) expressed as a percent of the control (C). T − C is the transgenic
hybrid combination minus the control. Oil, protein, and amino acid content are shown as percent of seed dry weight.

| | | Yield | | | | | | | | |
|---|---|---|---|---|---|---|---|---|---|---|
| | | (bu/a) | Oil (%) | Protein (%) | Arg (%) | Cys (%) | Lys (%) | Met (%) | Thr (%) | Val (%) |
| Event | Description | p < 0.1 | p < 0.05 | p < 0.05 | p < 0.05 | p < 0.05 | p < 0.05 | p < 0.05 | p < 0.05 | p < 0.05 |
| All | Construct (T) | 173.2 | 5.7 | 10.7 | 0.355 | 0.198 | 0.356 | 0.197 | 0.339 | 0.506 |
| All | Control (C) | 165.7 | 5.3 | 9.7 | 0.323 | 0.174 | 0.337 | 0.178 | 0.312 | 0.469 |
| All | T − C | 7.6 | 0.4 | 1.0 | 0.032 | 0.024 | 0.020 | 0.018 | 0.026 | 0.037 |
| All | (T/C)% | 105 | 108 | 110 | 110 | 114 | 106 | 110 | 108 | 108 |
| All | p-value | 0.03 | 0.00 | 0.00 | 0.00 | 0.00 | 0.01 | 0.00 | 0.00 | 0.00 |
| 1A | Event (T) | 167.9 | 5.7 | 10.8 | 0.356 | 0.195 | 0.358 | 0.197 | 0.342 | 0.512 |
| 1A | Control (C) | 167.0 | 5.3 | 9.8 | 0.326 | 0.175 | 0.337 | 0.179 | 0.316 | 0.473 |
| 1A | T − C | 0.9 | 0.4 | 1.1 | 0.030 | 0.019 | 0.021 | 0.018 | 0.026 | 0.039 |
| 1A | (T/C)% | 101 | 107 | 111 | 109 | 111 | 106 | 110 | 108 | 108 |
| 1A | p-value | 0.91 | 0.00 | 0.00 | 0.01 | 0.02 | 0.05 | 0.01 | 0.00 | 0.00 |
| 1B | Event (T) | 179.7 | 5.9 | 10.5 | 0.349 | 0.197 | 0.349 | 0.197 | 0.334 | 0.495 |
| 1B | Control (C) | 166.6 | 5.3 | 9.7 | 0.324 | 0.175 | 0.336 | 0.179 | 0.314 | 0.471 |
| 1B | T − C | 13.1 | 0.6 | 0.7 | 0.025 | 0.022 | 0.013 | 0.018 | 0.020 | 0.024 |
| 1B | (T/C)% | 108 | 112 | 108 | 108 | 112 | 104 | 110 | 106 | 105 |
| 1B | p-value | 0.01 | 0.00 | 0.00 | 0.03 | 0.01 | 0.22 | 0.01 | 0.01 | 0.04 |
| 1C | Event (T) | 169.6 | 5.7 | 10.9 | 0.369 | 0.211 | 0.354 | 0.207 | 0.346 | 0.518 |
| 1C | Control (C) | 166.8 | 5.3 | 9.7 | 0.322 | 0.174 | 0.336 | 0.178 | 0.312 | 0.468 |
| 1C | T − C | 2.9 | 0.5 | 1.2 | 0.046 | 0.037 | 0.018 | 0.029 | 0.034 | 0.050 |
| 1C | (T/C)% | 102 | 109 | 112 | 114 | 121 | 105 | 116 | 111 | 111 |
| 1C | p-value | 0.70 | 0.00 | 0.00 | 0.00 | 0.00 | 0.09 | 0.00 | 0.00 | 0.00 |
| 1D | Event (T) | 170.5 | 5.5 | 10.6 | 0.348 | 0.190 | 0.362 | 0.186 | 0.334 | 0.500 |
| 1D | Control (C) | 166.3 | 5.3 | 9.7 | 0.323 | 0.174 | 0.337 | 0.178 | 0.314 | 0.470 |
| 1D | T − C | 4.2 | 0.3 | 0.9 | 0.025 | 0.016 | 0.025 | 0.008 | 0.021 | 0.031 |
| 1D | (T/C)% | 103 | 105 | 109 | 108 | 109 | 107 | 105 | 107 | 107 |
| 1D | p-value | 0.41 | 0.01 | 0.00 | 0.01 | 0.03 | 0.01 | 0.16 | 0.00 | 0.00 |

TABLE 8
Summary of field data for Construct 1 by year. Numbers shown in bold are significantly different from the
control at the p-value shown. bu/a is bushels per acre. “All” indicates analysis across both years.
(T/C)% is the value for the transgenic hybrid combination (T) expressed as a percent of the control (C).

| | | Yield | | | | | | | | |
|---|---|---|---|---|---|---|---|---|---|---|
| | | (bu/a) | Oil (%) | Protein (%) | Arg (%) | Cys (%) | Lys (%) | Met (%) | Thr (%) | Val (%) |
| Year | Description | p < 0.1 | p < 0.05 | p < 0.05 | p < 0.05 | p < 0.05 | p < 0.05 | p < 0.05 | p < 0.05 | p < 0.05 |
| All | (T/C)% | 95 | 107 | 112 | 109 | 113 | 104 | 112 | 110 | 111 |
| 1 | (T/C)% | 97 | 104 | 111 | 108 | 110 | 101 | 108 | 109 | 110 |
| 2 | (T/C)% | 99 | 105 | 115 | 110 | 112 | 106 | 111 | 107 | 108 |

TABLE 9
Field data for Construct 1 by year. Numbers shown in bold are significantly different from the control at the p-value
shown. bu/a is bushels per acre. “All” indicates analysis across all events or both years. (T/C)% is the
value for the transgenic hybrid combination (T) expressed as a percent of the control (C). T − C is the transgenic
hybrid combination minus the control. Oil, protein, and amino acid content are shown as percent of seed dry weight.

| | | | Yield | | | | | | | | |
|---|---|---|---|---|---|---|---|---|---|---|---|
| | | | (bu/a) | Oil (%) | Protein (%) | Arg (%) | Cys (%) | Lys (%) | Met (%) | Thr (%) | Val (%) |
| Event | Year | Description | p < 0.1 | p < 0.05 | p < 0.05 | p < 0.05 | p < 0.05 | p < 0.05 | p < 0.05 | p < 0.05 | p < 0.05 |
| All | All | Construct (T) | 184.3 | 4.8 | 10.6 | 0.339 | 0.185 | 0.325 | 0.183 | 0.323 | 0.500 |
| All | All | Control (C) | 193.7 | 4.5 | 9.4 | 0.310 | 0.163 | 0.313 | 0.164 | 0.293 | 0.450 |
| All | All | T − C | −9.4 | 0.3 | 1.2 | 0.029 | 0.022 | 0.012 | 0.019 | 0.030 | 0.050 |
| All | All | (T/C)% | 95 | 107 | 112 | 109 | 113 | 104 | 112 | 110 | 111 |
| All | All | p-value | 0.01 | 0.00 | 0.00 | 0.00 | 0.00 | 0.08 | 0.00 | 0.00 | 0.00 |
| All | 1 | Construct (T) | 192.8 | 4.6 | 10.6 | 0.343 | 0.183 | 0.318 | 0.177 | 0.313 | 0.498 |
| All | 1 | Control (C) | 198.7 | 4.4 | 9.6 | 0.317 | 0.166 | 0.314 | 0.164 | 0.288 | 0.452 |
| All | 1 | T − C | −5.9 | 0.2 | 1.0 | 0.026 | 0.017 | 0.003 | 0.013 | 0.025 | 0.045 |
| All | 1 | (T/C)% | 97 | 104 | 111 | 108 | 110 | 101 | 108 | 109 | 110 |
| All | 1 | p-value | 0.03 | 0.01 | 0.00 | 0.00 | 0.02 | 0.62 | 0.00 | 0.00 | 0.00 |
| All | 2 | Construct (T) | 177.7 | 5.1 | 10.8 | 0.337 | 0.193 | 0.337 | 0.200 | 0.338 | 0.508 |
| All | 2 | Control (C) | 179.6 | 4.9 | 9.4 | 0.306 | 0.173 | 0.318 | 0.181 | 0.315 | 0.470 |
| All | 2 | T − C | −1.9 | 0.2 | 1.4 | 0.031 | 0.021 | 0.019 | 0.020 | 0.023 | 0.038 |
| All | 2 | (T/C)% | 99 | 105 | 115 | 110 | 112 | 106 | 111 | 107 | 108 |
| All | 2 | p-value | 0.69 | 0.00 | 0.00 | 0.04 | 0.05 | 0.11 | 0.05 | 0.03 | 0.05 |
| 1A | 1 | Event (T) | 196.7 | 4.6 | 10.5 | 0.338 | 0.169 | 0.323 | 0.174 | 0.311 | 0.493 |
| 1A | 1 | Control (C) | 198.7 | 4.4 | 9.6 | 0.318 | 0.167 | 0.316 | 0.164 | 0.288 | 0.452 |
| 1A | 1 | T − C | −2.0 | 0.3 | 0.9 | 0.020 | 0.003 | 0.007 | 0.010 | 0.023 | 0.041 |
| 1A | 1 | (T/C)% | 99 | 106 | 110 | 106 | 102 | 102 | 106 | 108 | 109 |
| 1A | 1 | p-value | 0.65 | 0.03 | 0.00 | 0.11 | 0.80 | 0.55 | 0.09 | 0.01 | 0.00 |
| 1B | ALL | Event (T) | 186.9 | 4.8 | 10.5 | 0.339 | 0.183 | 0.320 | 0.183 | 0.327 | 0.505 |
| 1B | ALL | Control (C) | 194.5 | 4.5 | 9.4 | 0.309 | 0.163 | 0.313 | 0.164 | 0.293 | 0.450 |
| 1B | ALL | T − C | −7.6 | 0.3 | 1.1 | 0.030 | 0.020 | 0.007 | 0.019 | 0.034 | 0.055 |
| 1B | ALL | (T/C)% | 96 | 107 | 112 | 110 | 112 | 102 | 112 | 112 | 112 |
| 1B | ALL | p-value | 0.11 | 0.01 | 0.00 | 0.01 | 0.02 | 0.44 | 0.00 | 0.00 | 0.00 |
| 1B | 1 | Event (T) | 189.5 | 4.5 | 10.4 | 0.341 | 0.187 | 0.310 | 0.176 | 0.317 | 0.503 |
| 1B | 1 | Control (C) | 198.7 | 4.4 | 9.5 | 0.317 | 0.165 | 0.315 | 0.164 | 0.288 | 0.452 |
| 1B | 1 | T − C | −9.2 | 0.1 | 0.9 | 0.024 | 0.022 | −0.004 | 0.012 | 0.029 | 0.052 |
| 1B | 1 | (T/C)% | 95 | 103 | 109 | 108 | 113 | 99 | 107 | 110 | 111 |
| 1B | 1 | p-value | 0.04 | 0.32 | 0.00 | 0.05 | 0.04 | 0.70 | 0.04 | 0.00 | 0.00 |
| 1B | 2 | Event (T) | 183.9 | 5.2 | 10.8 | 0.348 | 0.188 | 0.338 | 0.201 | 0.344 | 0.520 |
| 1B | 2 | Control (C) | 180.0 | 4.9 | 9.5 | 0.311 | 0.175 | 0.317 | 0.183 | 0.319 | 0.477 |
| 1B | 2 | T − C | 3.8 | 0.3 | 1.4 | 0.037 | 0.013 | 0.021 | 0.018 | 0.025 | 0.043 |
| 1B | 2 | (T/C)% | 102 | 107 | 115 | 112 | 107 | 107 | 110 | 108 | 109 |
| 1B | 2 | p-value | 0.51 | 0.00 | 0.00 | 0.02 | 0.23 | 0.16 | 0.09 | 0.04 | 0.05 |
| 1C | ALL | Event (T) | 182.8 | 4.8 | 10.7 | 0.344 | 0.192 | 0.326 | 0.190 | 0.325 | 0.503 |
| 1C | ALL | Control (C) | 192.7 | 4.5 | 9.4 | 0.310 | 0.163 | 0.314 | 0.164 | 0.293 | 0.451 |
| 1C | ALL | T − C | −9.9 | 0.3 | 1.3 | 0.034 | 0.029 | 0.012 | 0.026 | 0.032 | 0.052 |
| 1C | ALL | (T/C)% | 95 | 107 | 114 | 111 | 118 | 104 | 116 | 111 | 112 |
| 1C | ALL | p-value | 0.09 | 0.00 | 0.00 | 0.00 | 0.00 | 0.14 | 0.00 | 0.00 | 0.00 |
| 1C | 1 | Event (T) | 194.9 | 4.5 | 10.7 | 0.360 | 0.186 | 0.331 | 0.178 | 0.315 | 0.508 |
| 1C | 1 | Control (C) | 198.7 | 4.4 | 9.5 | 0.318 | 0.166 | 0.316 | 0.164 | 0.288 | 0.452 |
| 1C | 1 | T − C | −3.8 | 0.2 | 1.2 | 0.042 | 0.020 | 0.015 | 0.014 | 0.027 | 0.056 |
| 1C | 1 | (T/C)% | 98 | 104 | 112 | 113 | 112 | 105 | 108 | 109 | 112 |
| 1C | 1 | p-value | 0.37 | 0.16 | 0.00 | 0.00 | 0.06 | 0.13 | 0.01 | 0.00 | 0.00 |
| 1C | 2 | Event (T) | 176.3 | 5.2 | 10.9 | 0.332 | 0.209 | 0.324 | 0.214 | 0.339 | 0.505 |
| 1C | 2 | Control (C) | 180.4 | 4.9 | 9.3 | 0.303 | 0.171 | 0.317 | 0.179 | 0.314 | 0.468 |
| 1C | 2 | T − C | −4.1 | 0.3 | 1.6 | 0.029 | 0.037 | 0.007 | 0.035 | 0.026 | 0.038 |
| 1C | 2 | (T/C)% | 98 | 106 | 117 | 109 | 122 | 102 | 119 | 108 | 108 |
| 1C | 2 | p-value | 0.68 | 0.00 | 0.00 | 0.18 | 0.02 | 0.65 | 0.02 | 0.10 | 0.19 |
| 1D | ALL | Event (T) | 182.1 | 4.7 | 10.6 | 0.332 | 0.179 | 0.327 | 0.176 | 0.318 | 0.491 |
| 1D | ALL | Control (C) | 194.3 | 4.5 | 9.4 | 0.310 | 0.163 | 0.314 | 0.154 | 0.293 | 0.450 |
| 1D | ALL | T − C | −12.2 | 0.2 | 1.2 | 0.022 | 0.016 | 0.013 | 0.012 | 0.025 | 0.041 |
| 1D | ALL | (T/C)% | 94 | 104 | 112 | 107 | 110 | 104 | 107 | 109 | 109 |
| 1D | ALL | p-value | 0.02 | 0.02 | 0.00 | 0.06 | 0.08 | 0.16 | 0.08 | 0.00 | 0.01 |
| 1D | 1 | Event (T) | 190.2 | 4.6 | 10.6 | 0.336 | 0.188 | 0.313 | 0.180 | 0.307 | 0.485 |
| 1D | 1 | Control (C) | 198.7 | 4.4 | 9.5 | 0.318 | 0.166 | 0.316 | 0.154 | 0.288 | 0.452 |
| 1D | 1 | T − C | −8.5 | 0.2 | 1.1 | 0.019 | 0.022 | −0.003 | 0.016 | 0.019 | 0.033 |
| 1D | 1 | (T/C)% | 96 | 105 | 112 | 106 | 113 | 99 | 110 | 107 | 107 |
| 1D | 1 | p-value | 0.06 | 0.09 | 0.00 | 0.12 | 0.04 | 0.79 | 0.01 | 0.02 | 0.01 |
| 1D | 2 | Event (T) | 172.6 | 5.0 | 11.0 | 0.331 | 0.181 | 0.340 | 0.193 | 0.334 | 0.504 |
| 1D | 2 | Control (C) | 180.2 | 4.9 | 9.6 | 0.307 | 0.173 | 0.311 | −0.185 | 0.318 | 0.477 |
| 1D | 2 | T − C | −7.6 | 0.1 | 1.4 | 0.024 | 0.008 | 0.028 | 0.008 | 0.016 | 0.027 |
| 1D | 2 | (T/C)% | 96 | 103 | 115 | 108 | 105 | 109 | 104 | 105 | 106 |
| 1D | 2 | p-value | 0.20 | 0.09 | 0.01 | 0.24 | 0.53 | 0.10 | 0.57 | 0.28 | 0.31 |

TABLE 10
Summary of field data for Construct 2. Numbers shown in bold are significantly different from the control
at the p-value shown. bu/a is bushels per acre. “All” indicates analysis across all events. (T/C)%
is the value for the transgenic hybrid combination (T) expressed as a percent of the control (C).

| | | Yield | | | | | | | | |
|---|---|---|---|---|---|---|---|---|---|---|
| | | (bu/a) | Oil (%) | Prot (%) | Arg (%) | Cys (%) | Lys (%) | Met (%) | Thr (%) | Val (%) |
| Event | Desc. | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | (T/C)% | 95 | 104 | 102 | 100 | 100 | 99 | 101 | 100 | 100 |
| 2E | (T/C)% | 81 | n/a | n/a | n/a | n/a | n/a | n/a | n/a | n/a |
| 2F | (T/C)% | 79 | n/a | n/a | n/a | n/a | n/a | n/a | n/a | n/a |
| 2G | (T/C)% | 101 | 105 | 100 | 94 | 93 | 97 | 96 | 97 | 96 |
| 2H | (T/C)% | 103 | 102 | 99 | 95 | 97 | 96 | 100 | 97 | 98 |
| 2I | (T/C)% | 101 | 106 | 102 | 98 | 97 | 99 | 98 | 98 | 98 |
| 2J | (T/C)% | 103 | 104 | 103 | 103 | 100 | 105 | 101 | 102 | 102 |
| 2K | (T/C)% | 104 | 104 | 103 | 100 | 103 | 98 | 106 | 102 | 102 |

TABLE 11
Field data for Construct 2. Numbers shown in bold are significantly different from the control at the p-value
shown. bu/a is bushels per acre. “All” indicates analysis across all events. (T/C)% is the value for
the transgenic hybrid combination (T) expressed as a percent of the control (C). T − C is the transgenic
hybrid combination minus the control. Oil, protein, and amino acid content are shown as percent of seed dry weight.

| | | Yield | | | | | | | | |
|---|---|---|---|---|---|---|---|---|---|---|
| | Data | (bu/a) | Oil (%) | Prot (%) | Arg (%) | Cys (%) | Lys (%) | Met (%) | Thr (%) | Val (%) |
| Event | Description | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | Construct (T) | 174.9 | 4.8 | 9.5 | 0.310 | 0.162 | 0.326 | 0.168 | 0.298 | 0.458 |
| All | Control (C) | 183.9 | 4.6 | 9.3 | 0.311 | 0.163 | 0.328 | 0.166 | 0.296 | 0.457 |
| All | T − C | −9.0 | 0.2 | 0.2 | −0.001 | 0.000 | −0.003 | 0.002 | 0.001 | 0.001 |
| All | (T/C)% | 95 | 104 | 102 | 100 | 100 | 99 | 101 | 100 | 100 |
| All | p-value | 0.16 | 0.01 | 0.02 | 0.81 | 0.95 | 0.51 | 0.64 | 0.80 | 0.88 |
| 2E | Construct (T) | 148.1 | n/a | n/a | n/a | n/a | n/a | n/a | n/a | n/a |
| 2E | Control (C) | 183.9 | n/a | n/a | n/a | n/a | n/a | n/a | n/a | n/a |
| 2E | T − C | −35.8 | n/a | n/a | n/a | n/a | n/a | n/a | n/a | n/a |
| 2E | (T/C)% | 81 | n/a | n/a | n/a | n/a | n/a | n/a | n/a | n/a |
| 2E | p-value | 0.02 | n/a | n/a | n/a | n/a | n/a | n/a | n/a | n/a |
| 2F | Construct (T) | 145.6 | n/a | n/a | n/a | n/a | n/a | n/a | n/a | n/a |
| 2F | Control (C) | 183.9 | n/a | n/a | n/a | n/a | n/a | n/a | n/a | n/a |
| 2F | T − C | −38.3 | n/a | n/a | n/a | n/a | n/a | n/a | n/a | n/a |
| 2F | (T/C)% | 79 | n/a | n/a | n/a | n/a | n/a | n/a | n/a | n/a |
| 2F | p-value | 0.01 | n/a | n/a | n/a | n/a | n/a | n/a | n/a | n/a |
| 2G | Event (T) | 186.1 | 4.8 | 9.3 | 0.295 | 0.152 | 0.319 | 0.160 | 0.288 | 0.439 |
| 2G | Control (C) | 183.9 | 4.6 | 9.3 | 0.313 | 0.163 | 0.328 | 0.166 | 0.297 | 0.460 |
| 2G | T − C | 2.2 | 0.2 | 0.0 | −0.018 | −0.012 | −0.009 | −0.007 | −0.010 | −0.020 |
| 2G | (T/C)% | 101 | 105 | 100 | 94 | 93 | 97 | 96 | 97 | 96 |
| 2G | p-value | 0.67 | 0.16 | 0.77 | 0.09 | 0.44 | 0.46 | 0.44 | 0.36 | 0.12 |
| 2H | Event (T) | 189.2 | 4.7 | 9.2 | 0.297 | 0.156 | 0.315 | 0.166 | 0.290 | 0.450 |
| 2H | Control (C) | 183.9 | 4.6 | 9.3 | 0.312 | 0.160 | 0.329 | 0.167 | 0.298 | 0.458 |
| 2H | T − C | 5.3 | 0.1 | −0.1 | −0.015 | −0.004 | −0.013 | 0.000 | −0.008 | −0.007 |
| 2H | (T/C)% | 103 | 102 | 99 | 95 | 97 | 96 | 100 | 97 | 98 |
| 2H | p-value | 0.40 | 0.64 | 0.81 | 0.24 | 0.42 | 0.24 | 0.95 | 0.50 | 0.66 |
| 2I | Event (T) | 186.7 | 4.9 | 9.4 | 0.307 | 0.158 | 0.326 | 0.162 | 0.292 | 0.447 |
| 2I | Control (C) | 183.9 | 4.6 | 9.3 | 0.311 | 0.163 | 0.328 | 0.166 | 0.296 | 0.457 |
| 2I | T − C | 2.7 | 0.3 | 0.2 | −0.005 | −0.005 | −0.002 | −0.004 | −0.005 | −0.010 |
| 2I | (T/C)% | 101 | 106 | 102 | 98 | 97 | 99 | 98 | 98 | 98 |
| 2I | p-value | 0.60 | 0.01 | 0.54 | 0.64 | 0.37 | 0.73 | 0.66 | 0.69 | 0.49 |
| 2J | Event (T) | 189.4 | 4.8 | 9.6 | 0.322 | 0.163 | 0.346 | 0.168 | 0.301 | 0.468 |
| 2J | Control (C) | 183.9 | 4.6 | 9.3 | 0.311 | 0.163 | 0.328 | 0.166 | 0.296 | 0.457 |
| 2J | T − C | 5.5 | 0.2 | 0.3 | 0.010 | 0.000 | 0.018 | 0.002 | 0.005 | 0.011 |
| 2J | (T/C)% | 103 | 104 | 103 | 103 | 100 | 105 | 101 | 102 | 102 |
| 2J | p-value | 0.02 | 0.06 | 0.29 | 0.38 | 1.00 | 0.14 | 0.80 | 0.55 | 0.42 |
| 2K | Event (T) | 172.9 | 4.9 | 9.5 | 0.318 | 0.170 | 0.331 | 0.173 | 0.303 | 0.473 |
| 2K | Control (C) | 165.6 | 4.7 | 9.2 | 0.319 | 0.165 | 0.338 | 0.163 | 0.296 | 0.465 |
| 2K | T − C | 7.3 | 0.2 | 0.3 | 0.000 | 0.005 | −0.007 | 0.010 | 0.007 | 0.008 |
| 2K | (T/C)% | 104 | 104 | 103 | 100 | 103 | 98 | 106 | 102 | 102 |
| 2K | p-value | 0.49 | 0.07 | 0.50 | 0.98 | 0.78 | 0.33 | 0.44 | 0.64 | 0.69 |

TABLE 12
Summary of field data for Construct 3. Numbers shown in bold are significantly different from the control
at the p-value shown. bu/a is bushels per acre. “All” indicates analysis across all events. (T/C)%
is the value for the transgenic hybrid combination (T) expressed as a percent of the control (C).

| | | Yield | | | | | | | | |
|---|---|---|---|---|---|---|---|---|---|---|
| | Data | (bu/a) | Oil (%) | Prot (%) | Arg (%) | Cys (%) | Lys (%) | Met (%) | Thr (%) | Val (%) |
| Event | Description | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | (T/C)% | 96 | 101 | 105 | 106 | 108 | 105 | 102 | 104 | 105 |
| 3L | (T/C)% | 96 | 108 | 104 | 109 | 116 | 108 | 107 | 104 | 106 |
| 3M | (T/C)% | 97 | 105 | 103 | 101 | 99 | 105 | 95 | 98 | 102 |
| 3N | (T/C)% | 95 | 97 | 109 | 109 | 120 | 107 | 107 | 108 | 108 |
| 3O | (T/C)% | 97 | 103 | 105 | 102 | 107 | 99 | 102 | 101 | 103 |
| 3P | (T/C)% | 95 | 102 | 104 | 110 | 108 | 108 | 99 | 107 | 107 |
| 3Q | (T/C)% | 97 | 100 | 103 | 105 | 101 | 105 | 101 | 103 | 104 |

TABLE 13
Field data for Construct 3. Numbers shown in bold are significantly different from the control at the p-value
shown. bu/a is bushels per acre. “All” indicates analysis across all events. (T/C)% is the value for
the transgenic hybrid combination (T) expressed as a percent of the control (C). T − C is the transgenic
hybrid combination minus the control. Oil, protein, and amino acid content are shown as percent of seed dry weight.

| | | Yield | | | | | | | | |
|---|---|---|---|---|---|---|---|---|---|---|
| | Data | (bu/a) | Oil (%) | Prot (%) | Arg (%) | Cys (%) | Lys (%) | Met (%) | Thr (%) | Val (%) |
| Event | Description | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | Construct (T) | 185.1 | 4.7 | 9.6 | 0.307 | 0.143 | 0.319 | 0.162 | 0.293 | 0.451 |
| All | Control (C) | 191.9 | 4.6 | 9.2 | 0.290 | 0.132 | 0.305 | 0.159 | 0.283 | 0.432 |
| All | T − C | −6.8 | 0.1 | 0.4 | 0.017 | 0.011 | 0.014 | 0.003 | 0.010 | 0.020 |
| All | (T/C)% | 96 | 101 | 105 | 106 | 108 | 105 | 102 | 104 | 105 |
| All | p-value | 0.00 | 0.30 | 0.02 | 0.09 | 0.29 | 0.14 | 0.40 | 0.12 | 0.06 |
| 3L | Event (T) | 186.1 | 4.8 | 9.5 | 0.317 | 0.151 | 0.329 | 0.169 | 0.298 | 0.459 |
| 3L | Control (C) | 193.1 | 4.5 | 9.1 | 0.291 | 0.130 | 0.305 | 0.158 | 0.288 | 0.432 |
| 3L | T − C | −7.0 | 0.3 | 0.4 | 0.026 | 0.021 | 0.024 | 0.011 | 0.010 | 0.028 |
| 3L | (T/C)% | 96 | 108 | 104 | 109 | 116 | 108 | 107 | 104 | 106 |
| 3L | p-value | 0.12 | 0.15 | 0.13 | 0.25 | 0.23 | 0.06 | 0.19 | 0.48 | 0.15 |
| 3M | Event (T) | 187.3 | 4.8 | 9.4 | 0.296 | 0.131 | 0.319 | 0.152 | 0.282 | 0.441 |
| 3M | Control (C) | 192.2 | 4.5 | 9.2 | 0.292 | 0.133 | 0.304 | 0.159 | 0.287 | 0.434 |
| 3M | T − C | −4.9 | 0.2 | 0.3 | 0.003 | −0.002 | 0.015 | −0.007 | −0.005 | 0.007 |
| 3M | (T/C)% | 97 | 105 | 103 | 101 | 99 | 105 | 95 | 98 | 102 |
| 3M | p-value | 0.16 | 0.30 | 0.08 | 0.85 | 0.88 | 0.47 | 0.31 | 0.74 | 0.67 |
| 3N | Event (T) | 182.8 | 4.6 | 9.9 | 0.311 | 0.153 | 0.325 | 0.165 | 0.302 | 0.460 |
| 3N | Control (C) | 191.9 | 4.7 | 9.1 | 0.286 | 0.127 | 0.305 | 0.155 | 0.279 | 0.427 |
| 3N | T − C | −9.1 | −0.1 | 0.8 | 0.025 | 0.026 | 0.020 | 0.011 | 0.023 | 0.033 |
| 3N | (T/C)% | 95 | 97 | 109 | 109 | 120 | 107 | 107 | 108 | 108 |
| 3N | p-value | 0.02 | 0.02 | 0.11 | 0.20 | 0.10 | 0.30 | 0.05 | 0.08 | 0.10 |
| 3O | Event (T) | 180.3 | 4.8 | 9.7 | 0.296 | 0.141 | 0.303 | 0.162 | 0.286 | 0.443 |
| 3O | Control (C) | 186.3 | 4.6 | 9.2 | 0.290 | 0.132 | 0.305 | 0.159 | 0.283 | 0.432 |
| 3O | T − C | −6.0 | 0.1 | 0.5 | 0.007 | 0.009 | −0.002 | 0.003 | 0.004 | 0.011 |
| 3O | (T/C)% | 97 | 103 | 105 | 102 | 107 | 99 | 102 | 101 | 103 |
| 3O | p-value | 0.16 | 0.02 | 0.01 | 0.53 | 0.53 | 0.88 | 0.58 | 0.61 | 0.24 |
| 3P | Event (T) | 182.8 | 4.6 | 9.5 | 0.320 | 0.143 | 0.328 | 0.158 | 0.308 | 0.463 |
| 3P | Control (C) | 193.4 | 4.5 | 9.2 | 0.292 | 0.133 | 0.304 | 0.159 | 0.287 | 0.434 |
| 3P | T − C | −10.6 | 0.1 | 0.4 | 0.028 | 0.010 | 0.024 | −0.001 | 0.021 | 0.029 |
| 3P | (T/C)% | 95 | 102 | 104 | 110 | 108 | 108 | 99 | 107 | 107 |
| 3P | p-value | 0.02 | 0.41 | 0.13 | 0.02 | 0.56 | 0.03 | 0.81 | 0.00 | 0.08 |
| 3Q | Event (T) | 186.8 | 4.6 | 9.4 | 0.301 | 0.131 | 0.317 | 0.154 | 0.287 | 0.441 |
| 3Q | Control (C) | 191.9 | 4.6 | 9.1 | 0.287 | 0.131 | 0.301 | 0.152 | 0.278 | 0.426 |
| 3Q | T − C | −5.1 | 0.0 | 0.3 | 0.014 | 0.001 | 0.016 | 0.002 | 0.009 | 0.015 |
| 3Q | (T/C)% | 97 | 100 | 103 | 105 | 101 | 105 | 101 | 103 | 104 |
| 3Q | p-value | 0.18 | 1.00 | 0.21 | 0.05 | 0.92 | 0.03 | 0.51 | 0.05 | 0.17 |

TABLE 14
Summary of field data for Construct 4. Numbers shown in bold are significantly different from the control
at the p-value shown. bu/a is bushels per acre. “All” indicates analysis across all events. (T/C)%
is the value for the transgenic hybrid combination (T) expressed as a percent of the control (C).

| | | Yield | | | | | | | | |
|---|---|---|---|---|---|---|---|---|---|---|
| | Data | (bu/a) | Oil (%) | Prot (%) | Arg (%) | Cys (%) | Lys (%) | Met (%) | Thr (%) | Val (%) |
| Event | Description | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | (T/C)% | 96 | 102 | 103 | 100 | 105 | 99 | 102 | 101 | 101 |
| 4R | (T/C)% | 95 | 105 | 104 | 98 | 106 | 97 | 102 | 102 | 103 |
| 4S | (T/C)% | 82 | 102 | 113 | 107 | 126 | 101 | 113 | 106 | 107 |
| 4T | (T/C)% | 99 | 100 | 98 | 97 | 91 | 98 | 99 | 98 | 98 |
| 4U | (T/C)% | 102 | 103 | 101 | 99 | 101 | 100 | 98 | 100 | 100 |
| 4V | (T/C)% | 98 | 104 | 102 | 97 | 105 | 96 | 102 | 100 | 99 |

TABLE 15
Field data for Construct 4. Numbers shown in bold are significantly different from the control at the p-value
shown. bu/a is bushels per acre. “All” indicates analysis across all events. (T/C)% is the value for
the transgenic hybrid combination (T) expressed as a percent of the control (C). T − C is the transgenic
hybrid combination minus the control. Oil, protein, and amino acid content are shown as percent of seed dry weight.

| | | Yield | | | | | | | | |
|---|---|---|---|---|---|---|---|---|---|---|
| | Data | (bu/a) | Oil (%) | Prot (%) | Arg (%) | Cys (%) | Lys (%) | Met (%) | Thr (%) | Val (%) |
| Event | Description | p ≦ 0.10 | p ≦ 0.15 | p ≦ 0.15 | p ≦ 0.15 | p ≦ 0.15 | p ≦ 0.15 | p ≦ 0.15 | p ≦ 0.15 | p ≦ 0.15 |
| All | Construct (T) | 195.1 | 4.7 | 9.4 | 0.315 | 0.160 | 0.316 | 0.166 | 0.305 | 0.445 |
| All | Control (C) | 203.4 | 4.6 | 9.1 | 0.316 | 0.153 | 0.320 | 0.163 | 0.301 | 0.439 |
| All | T − C | −8.4 | 0.1 | 0.3 | −0.001 | 0.007 | −0.003 | 0.003 | 0.004 | 0.006 |
| All | (T/C)% | 96 | 102 | 103 | 100 | 105 | 99 | 102 | 101 | 101 |
| All | p-value | 0.22 | 0.03 | 0.28 | 0.89 | 0.35 | 0.72 | 0.35 | 0.33 | 0.36 |
| 4R | Event (T) | 193.8 | 4.9 | 9.4 | 0.311 | 0.162 | 0.312 | 0.164 | 0.307 | 0.450 |
| 4R | Control (C) | 203.4 | 4.6 | 9.1 | 0.316 | 0.152 | 0.322 | 0.161 | 0.301 | 0.437 |
| 4R | T − C | −9.6 | 0.2 | 0.3 | −0.005 | 0.009 | −0.010 | 0.003 | 0.007 | 0.013 |
| 4R | (T/C)% | 95 | 105 | 104 | 98 | 106 | 97 | 102 | 102 | 103 |
| 4R | p-value | 0.04 | 0.02 | 0.31 | 0.62 | 0.33 | 0.50 | 0.54 | 0.16 | 0.14 |
| 4S | Event (T) | 166.4 | 4.7 | 10.3 | 0.337 | 0.190 | 0.325 | 0.184 | 0.321 | 0.472 |
| 4S | Control (C) | 203.6 | 4.6 | 9.1 | 0.316 | 0.150 | 0.321 | 0.163 | 0.302 | 0.440 |
| 4S | T − C | −37.2 | 0.1 | 1.2 | 0.021 | 0.039 | 0.005 | 0.022 | 0.019 | 0.032 |
| 4S | (T/C)% | 82 | 102 | 113 | 107 | 126 | 101 | 113 | 106 | 107 |
| 4S | p-value | 0.02 | 0.02 | 0.05 | 0.03 | 0.09 | 0.69 | 0.05 | 0.01 | 0.01 |
| 4T | Event (T) | 202.4 | 4.6 | 8.9 | 0.309 | 0.141 | 0.316 | 0.161 | 0.297 | 0.430 |
| 4T | Control (C) | 203.4 | 4.6 | 9.1 | 0.318 | 0.155 | 0.322 | 0.162 | 0.302 | 0.440 |
| 4T | T − C | −1.0 | 0.0 | −0.2 | −0.009 | −0.013 | −0.006 | −0.001 | −0.005 | −0.010 |
| 4T | (T/C)% | 99 | 100 | 98 | 97 | 91 | 98 | 99 | 98 | 98 |
| 4T | p-value | 0.85 | 0.78 | 0.52 | 0.14 | 0.17 | 0.49 | 0.40 | 0.32 | 0.22 |
| 4U | Event (T) | 207.0 | 4.8 | 9.2 | 0.312 | 0.155 | 0.320 | 0.160 | 0.300 | 0.437 |
| 4U | Control (C) | 203.4 | 4.6 | 9.1 | 0.316 | 0.153 | 0.320 | 0.163 | 0.301 | 0.439 |
| 4U | T − C | 3.6 | 0.1 | 0.1 | −0.004 | 0.002 | 0.001 | −0.003 | −0.001 | −0.002 |
| 4U | (T/C)% | 102 | 103 | 101 | 99 | 101 | 100 | 98 | 100 | 100 |
| 4U | p-value | 0.68 | 0.05 | 0.80 | 0.57 | 0.89 | 0.97 | 0.42 | 0.85 | 0.91 |
| 4V | Event (T) | 199.6 | 4.8 | 9.3 | 0.307 | 0.160 | 0.307 | 0.167 | 0.301 | 0.436 |
| 4V | Control (C) | 203.4 | 4.6 | 9.1 | 0.316 | 0.153 | 0.320 | 0.163 | 0.301 | 0.439 |
| 4V | T − C | −3.8 | 0.2 | 0.1 | −0.009 | 0.007 | −0.013 | 0.004 | 0.000 | −0.003 |
| 4V | (T/C)% | 98 | 104 | 102 | 97 | 105 | 96 | 102 | 100 | 99 |
| 4V | p-value | 0.73 | 0.03 | 0.70 | 0.71 | 0.65 | 0.50 | 0.69 | 0.97 | 0.89 |

1. An expression cassette comprising:
(a) a promoter that is functional in a plant;
(b) a nucleic acid molecule; and
(c) the first intron of the rice Metallothionein) gene (Met1-1),
wherein the nucleic acid molecule is operably linked to the promoter, and expression of the nucleic acid molecule in a plant, plant cell, or plant part confers an increase in one or more of protein, oil, or one or more amino acids in a plant, plant cell, or plant part relative to a corresponding wild-type plant, plant cell, or plant part; and
wherein the nucleic acid molecule comprises:
(i) the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23,
SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49.

2. An expression cassette comprising:
(a) an ScBV promoter or a functional fragment thereof;
(b) a nucleic acid molecule; and
(c) an intron,
wherein the nucleic acid molecule is operably linked to the promoter, and expression of the nucleic acid molecule in a plant, plant cell, or plant part confers an increase in one or more of protein, oil, or one or more amino acids in a plant, plant cell, or plant part relative to a corresponding wild-type plant, plant cell, or plant part; and
wherein the nucleic acid molecule comprises:
(i) the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 and SEQ ID NO: 49.

3. An expression cassette comprising:
(a) a promoter that is functional in a plant; and
(b) a nucleic acid molecule,
wherein the nucleic acid molecule is operably linked to the promoter, and expression of the nucleic acid molecule in a plant, plant cell, or plant part confers an increase in protein and one or more amino acids in a plant, plant cell, or plant part relative to a corresponding wild-type plant, plant cell, or plant part; and
wherein the nucleic acid molecule comprises:
(i) the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51;
(ii) a nucleotide sequence encoding the amino acid sequence of SEQ ID NO: 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37;
(iii) a nucleotide sequence having at least 70% sequence identity to the nucleotide sequence of SEQ ID NO: 1, SEQ ID NO: 3, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 50, or SEQ ID NO: 51 and encoding a polypeptide having a Pfam:PF00982.15 glycosyltransferase family 20 domain and a Pfam:PF02358.10 trehalose-phosphatase domain;
(iv) a nucleotide sequence encoding an amino acid sequence having at least 80% sequence identity to the amino acid sequence of SEQ ID NO 2, SEQ ID NO: 4, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, or SEQ ID NO: 37 and having a Pfam:PF00982.15 glycosyltransferase family 20 domain and a Pfam:PF02358.10 trehalose-phosphatase domain;
(v) a nucleotide sequence encoding an amino acid sequence comprising a Pfam:PF00982.15 glycosyltransferase family 20 domain and a Pfam:PF02358.10 trehalose-phosphatase domain, wherein the Pfam:PF00982.15 glycosyltransferase family 20 domain has at least 50% sequence identity to amino acid residues 57 to 541 of SEQ ID NO: 2, amino acid residues 59 to 546 of SEQ ID NO: 4, amino acid residues 60 to 546 of SEQ ID NO: 17, amino acid residues 50 to 538 of SEQ ID NO: 19, amino acid residues 59 to 546 of SEQ ID NO: 21, amino acid residues 23 to 511 of SEQ ID NO: 23, amino acid residues 77 to 562 of SEQ ID NO: 25, amino acid residues 59 to 550 of SEQ ID NO: 27, amino acid residues 61 to 546 of SEQ ID NO: 29, amino acid residues 2 to 462 of SEQ ID NO: 31, amino acid residues 22 to 514 of SEQ ID NO: 33, amino acid residues 59 to 546 of SEQ ID NO: 35, or amino acid residues 58 to 541 of SEQ ID NO: 37, and wherein the Pfam:PF02358.10 trehalose-phosphatase domain has at least 55% sequence identity to amino acid residues 590 to 825 of SEQ ID NO: 2, amino acid residues 595 to 830 of SEQ ID NO: 4, amino acid residues 595 to 830 of SEQ ID NO: 17, amino acid residues 587 to 822 of SEQ ID NO: 19, amino acid residues 595 to 830 of SEQ ID NO: 21, amino acid residues 560 to 794 of SEQ ID NO: 23, amino acid residues 611 to 846 of SEQ ID NO: 25, amino acid residues 599 to 832 of SEQ ID NO: 27, amino acid residues 595 to 830 of SEQ ID NO: 29, amino acid residues 496 to 714 of SEQ ID NO: 31, amino acid residues 546 to 782 of SEQ ID NO: 33, amino acid residues 595 to 830 of SEQ ID NO: 35, or amino acid residues 590 to 825 of SEQ ID NO: 37; or
(vi) a nucleotide sequence encoding an amino acid sequence comprising the amino acid sequence of SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49.

4. The expression cassette of claim 1, wherein the promoter is a constitutive promoter, a seed-preferred promoter, or a seed-specific promoter.

5. The expression cassette of claim 1, wherein the promoter is a constitutive promoter and comprises:
(a) the nucleic acid sequence of SEQ ID NO: 8 or SEQ ID NO: 9;
(b) a nucleic acid sequence having at least 95% sequence identity to the nucleic acid sequence of SEQ ID NO: 8 or SEQ ID NO: 9, wherein said nucleic acid sequence has constitutive expression activity; or
(c) a fragment of the nucleic acid sequence of SEQ ID NO: 8 or SEQ ID NO: 9, wherein the fragment has constitutive expression activity.

6. The expression cassette of claim 1, wherein the promoter is an embryo-specific promoter and comprises:
(a) the nucleic acid sequence of SEQ ID NO: 7;
(b) a nucleic acid sequence having at least 95% sequence identity to the nucleic acid sequence of SEQ ID NO: 7, wherein said nucleic acid sequence has embryo-specific expression activity; or
(c) a fragment of the nucleic acid sequence of SEQ ID NO: 7, wherein the fragment has embryo-specific expression activity.

7. The expression cassette of claim 3, further comprising an intron.

8. The expression cassette of claim 2, wherein the intron is a monocot intron.

9. The expression cassette of claim 1, wherein the intron comprises the nucleic acid sequence of SEQ ID NO: 10 or a nucleic acid sequence having at least 90% sequence identity to SEQ ID NO: 10.

10. The expression cassette of claim 1, further comprising a nucleic acid sequence encoding a transit peptide that targets the polypeptide to a plastid.

11. The expression cassette of claim 10, wherein the transit peptide is a plastid-targeting peptide from a ferredoxin gene.

12. The expression cassette of claim 10, wherein the nucleic acid sequence encoding a transit peptide comprises:
(a) the nucleic acid sequence of SEQ ID NO: 5 or 73;
(b) a nucleic acid sequence encoding SEQ ID NO: 6;
(c) a nucleic acid sequence having at least 95% sequence identity to SEQ ID NO: 5 or 73; or
(d) a nucleic acid sequence encoding a polypeptide having at least 95% sequence identity to SEQ ID NO: 6.

13. The expression cassette of claim 1, further comprising a terminator.

14. The expression cassette of claim 13, wherein the terminator is a NOS terminator or comprises the nucleic acid sequence of SEQ ID NO: 11.

15. The expression cassette of claim 1, wherein the promoter comprises the nucleic acid sequence of SEQ ID NO: 8, the nucleic acid molecule comprises the nucleic acid sequence of SEQ ID NO: 3, and the intron comprises the nucleic acid sequence of SEQ ID NO: 10, and wherein the expression cassette further comprises the nucleic acid sequence of SEQ ID NO: 11.

16. The expression cassette of claim 1, wherein the expression cassette confers an increase in oil in a plant, plant cell, or plant part relative to a corresponding wild-type plant, plant cell, or plant part.

17. A recombinant construct comprising at least one expression cassette of claim 1.

18. A vector comprising at least one expression cassette of claim 1 or a recombinant construct comprising the expression cassette.

19. A microorganism comprising the expression cassette of claim 1, a recombinant construct comprising the expression cassette, or a vector comprising theme expression cassette or the recombinant construct.

20. A plant, plant cell, or plant part comprising the expression cassette of claim 1 or a recombinant construct comprising the expression cassette, wherein the plant, plant cell, or plant part has an increase in one or more of protein, oil, or one or more amino acids relative to a corresponding wild-type plant, plant cell, or plant part.

21. A plant, plant cell, or plant part comprising the expression cassette of claim 1 or a recombinant construct comprising the expression cassette, wherein the plant, plant cell, or plant part has an increase in protein and one or more amino acids relative to a corresponding wild-type plant, plant cell, or plant part.

22. A plant, plant cell, or plant part comprising the expression cassette of claim 1 or a recombinant construct comprising the expression cassette, wherein the plant, plant cell, or plant part has an increase in protein, oil, and one or more amino acids relative to a corresponding wild-type plant, plant cell, or plant part.

23. The plant part of claim 20, wherein the plant part is a seed.

24. A food or feed composition comprising the plant, plant cell, or plant part of claim 20.

25. The food or feed composition of claim 24, wherein the food or feed composition is not supplemented with additional protein, oil, or one or more amino acids or has reduced supplementation with protein, oil, or one or more amino acids relative to a food or feed composition comprising a corresponding wild-type plant, plant cell, or plant part.

26. The feed composition of claim 24, wherein the feed composition is formulated to meet the dietary requirements of swine, poultry, cattle, companion animals, or fish.

27. A method for producing a transgenic plant, plant cell, or plant part having an increase in one or more of protein, oil or one or more amino acids, comprising
(a) transforming a plant, plant cell, or plant part with the expression cassette of claim 1, a recombinant construct comprising the expression cassette, or a vector comprising the expression cassette or the recombinant construct; and
(b) optionally regenerating from the plant, plant cell, or plant part a transgenic plant,
wherein the transgenic plant, plant cell, or plant part has increased content of protein, oil, and/or one or more amino acids relative to a corresponding wild-type plant, plant cell, or plant part.

28. A method for increasing one or more of protein, oil, or one or more amino acids in a plant, plant cell, or plant part relative to a corresponding wild-type plant, plant cell, or plant part, comprising:
(a) obtaining the plant, plant cell, or plant part of claim 20; and
(b) selecting a plant, plant cell, or plant part with an increase in one or more of protein, oil, or one or more amino acids.

29. The method of claim 28, wherein the plant is a monocotyledonous plant or the plant cell or plant part is from a monocotyledonous plant.

30. The method of claim 27, wherein the content of one or more amino acids in said plant, plant cell, or plant part is increased relative to a corresponding wild-type plant, plant cell, or plant part, and the one or more amino acids is selected from the group consisting of arginine, cysteine, lysine, methionine, threonine, and valine.

31. The method of claim 30, wherein the content of two or more amino acids is increased.

32. The method of claim 27, wherein the content of protein in said plant, plant cell, or plant part is increased relative to a corresponding wild-type plant, plant cell, or plant part.

33. The method of claim 27, wherein the content of oil in said plant, plant cell, or plant part is increased relative to a corresponding wild-type plant, plant cell, or plant part.

34. A method of producing a food or feed composition comprising
(a) providing a plant, plant cell, or plant part comprising the expression cassette of claim 1 or a recombinant construct comprising the expression cassette; and
(b) producing a food or feed composition comprising the plant, plant cell or plant part.

35. A method for producing a hybrid maize plant or seed comprising crossing a first inbred parent maize plant with a second inbred parent maize plant and harvesting a resultant hybrid maize seed, wherein said first inbred parent maize plant or said second inbred parent maize plant comprises the expression cassette of claim 1 or a recombinant construct comprising the expression cassette, or wherein said first inbred parent maize plant or said second inbred parent maize plant is derived from a plant that comprises the expression cassette of claim 1 or a recombinant construct comprising the expression cassette.

36. A method for developing a maize plant or seed in a maize plant breeding program using plant breeding techniques comprising employing a maize plant or part thereof as a source of plant breeding material, wherein the maize plant or part thereof comprises the expression cassette of claim 1 or a recombinant construct comprising the expression cassette.

37. The method of claim 36, wherein the plant breeding techniques are selected from the group consisting of recurrent selection, backcrossing, pedigree breeding, restriction length polymorphism enhanced selection, genetic marker enhanced selection, and transformation techniques.

38. A hybrid maize plant or seed produced by the method of claim 35.

39. A maize plant or part thereof produced by growing the seed of claim 38.

40. A method of plant breeding comprising:
(a) obtaining the hybrid maize seed of claim 38;
(b) crossing a plant grown from the hybrid maize seed with a different maize plant; and
(c) selecting a resultant progeny having an increase in one or more of protein, oil, or one or more amino acids.

41. A method for producing grain with an increase in one or more of protein, oil, or one or more amino acids, comprising:
(a) obtaining a seed of a first plant and a seed of a second plant, the first plant comprising the expression cassette of claim 1 or a recombinant construct comprising the expression cassette;
(b) growing the seed under conditions that result in cross pollination between the plant produced from the seed of the first plant and the plant produced from the seed of the second plant; and
(c) harvesting grain from the progeny.

42. Grain produced by the method of claim 41, wherein the grain has an increase in one or more of protein, oil or one or more amino acids relative to a corresponding wild-type grain.

43. The grain of claim 42, wherein the one or more amino acids is selected from the group consisting of arginine, cysteine, lysine, methionine, threonine, and valine.

44. The grain of claim 42, wherein the grain is corn.

45. A method of producing a maize plant with an increase in one or more of protein, oil, or one or more amino acids, comprising:
(a) growing a progeny plant produced by crossing a maize plant comprising the expression cassette of claim 1 or a recombinant construct comprising the expression cassette with a second maize plant;
(b) crossing the progeny plant with itself or a different plant to produce a seed of a progeny plant of a subsequent generation;
(c) growing a progeny plant of a subsequent generation from said seed and crossing the progeny plant of a subsequent generation with itself or a different plant; and
(d) repeating steps (b) and (c) for an additional 0-5 generations to produce a maize plant.

46. The method of claim 45, wherein the produced maize plant is an inbred maize plant.

47. The method of claim 46, further comprising crossing the inbred maize plant with a second, distinct inbred maize plant to produce an F1 hybrid maize plant.


SEQUENCE LISTING (77 sequences)

SEQ ID NO: 1 - DNA, 2571 bp, Arabidopsis thaliana
atggtgtcaagatcttgtgctaattttctagacttatcatcttgggaccttttagattttcctcaaactccacgaactcttccacgcgtcatgactgttccgggaatcatcaccgacgtagacggtgatacaacctccgaagtaacttctacctccggtggttcacgtgagaggaagatcattgtagctaacatgttaccactccaatctaaaagagatgcagaaactggtaaatggtgttttaactgggacgaagactctctccagttacaacttagagatgggttctcttcagaaacagagtttctctacgttggatcacttaacgtagacatcgaaactaacgaacaagaagaagtttcacagaagcttttagaggaatttaactgcgttgcaacgtttttgtctcaagagttgcaagaaatgttctatcttggtttctgtaaacatcagttatggccactctttcattacatgcttccaatgtttcctgatcatggtgatcgttttgatcgacgtctatggcaagcttatgtatcagccaacaagatattttcagatagagttatggaagttatcaaccctgaggatgattacgtttggattcaagattatcatcttatggttcttcctactttcttgaggaaacgttttaataggatcaaactcggtttcttccttcatagtccgtttccttcttcagagatttaccgcacattgcctgttcgtgacgagattttgagaggtttgttgaattgtgatctcattggtttccatacgtttgattacgcgaggcatttcttgtcttgttgtagtagaatgcttggtcttgattacgagtctaagcgcggtcacataggtcttgattactttggtaggactgtgtatatcaaaatacttcctgttggtgttcatatgggtagattggagtctgttttgagtcttgattctactgcggcgaagacgaaagagattcaagaacagtttaaagggaagaagcttgttcttggtatcgatgatatggatatatttaaagggataagcttaaagcttatagcaatggaacatctctttgagacttattggcatttgaaagggaaagttgttcttgttcagatagtgaaccctgcaagatcttctggtaaagatgttgaagaagcgaagagggagacgtatgagactgcgaggaggatcaatgagcgttacggtacttctgactataagccgatagttttgatcgatcgtcttgttccacgttctgagaaaaccgcgtattatgctgcagcagattgttgcttagtgaatgcagtgagagatggtatgaacttagttccttataagtatatcgtctgcaggcaggggactcgaagtaataaggccgttgtggattcatcgcctcgcacaagcactcttgtcgtgtctgagtttattggatgctcaccttctttgagtggtgccattagggtgaatccatgggatgtggatgctgttgctgaagcggtaaactcggctcttaaaatgagtgagactgagaagcaactacggcatgagaaacattatcattatattagcactcatgatgttggttattgggcaaagagctttatgcaggatcttgagagagcgtgccgagatcattatagtaaacgttgttgggggattggttttggtttggggttcagagttttgtcactctctccaagttttaggaagctatctgtggaacacattgttccagtttatagaaaaacacagagaagagctatatttcttgattatgatggtactcttgttcctgaaagctccattgttcaagatccaagcaacgaggttgtctctgttctgaaagctctctgtgaagatccgaataacacggtgtttattgttagtggaagaggtagagagtctctgagcaattggctatctccttgtgaaaatcttggaatagcagctgaacatggatacttcattagatggaagagcaaagatgagtgggagacttgttattcgcctacggatacagagtggaggtcaatggtggaaccggttatgagatcgtatatggaggcaacagatgggacgagtatagagtttaaagaaagtgctttggtgtggcaccatcaagacgcagatcctgactttggatcatgtcaagctaaggagatgcttgatcatctagagagtgttctcgccaatgagcctgtggttgtcaagagaggtcaacacatcgttgaagtcaaaccacaaggtgtaagcaaaggtctagctgcggagaaagtaatccgagaaatggtagaacgcggggagccaccggaaatggtgatgtgcataggagacgatagatcagacgaagacatgtttgagagcatattaagcacagtgacaaatccggaacttcttgtgcagccagaggtttttgcatgcacggttggaagaaaaccaagcaaagctaaatacttcttggacgatgaagccgacgtgcttaagctcctaagaggtcttggagactcatcatcgagcttaaaacctagttcttctcacacacaagttgcatttgaaagcatcgtttaa

SEQ ID NO: 2 - protein, 856 aa, Arabidopsis thaliana
MVSRSCANFLDLSSWDLLDFPQTPRTLPRVMTVPGIITDVDGDTTSEVTSTSGGSRERKIIVANMLPLQSKRDAETGKWCFNWDEDSLQLQLRDGFSSETEFLYVGSLNVDIETNEQEEVSQKLLEEFNCVATFLSQELQEMFYLGFCKHQLWPLFHYMLPMFPDHGDRFDRRLWQAYVSANKIFSDRVMEVINPEDDYVWIQDYHLMVLPTFLRKRFNRIKLGFFLHSPFPSSEIYRTLPVRDEILRGLLNCDLIGFHTFDYARHFLSCCSRMLGLDYESKRGHIGLDYFGRTVYIKILPVGVHMGRLESVLSLDSTAAKTKEIQEQFKGKKLVLGIDDMDIFKGISLKLIAMEHLFETYWHLKGKVVLVQIVNPARSSGKDVEEAKRETYETARRINERYGTSDYKPIVLIDRLVPRSEKTAYYAAADCCLVNAVRDGMNLVPYKYIVCRQGTRSNKAVVDSSPRTSTLVVSEFIGCSPSLSGAIRVNPWDVDAVAEAVNSALKMSETEKQLRHEKHYHYISTHDVGYWAKSFMQDLERACRDHYSKRCWGIGFGLGFRVLSLSPSFRKLSVEHIVPVYRKTQRRAIFLDYDGTLVPESSIVQDPSNEVVSVLKALCEDPNNTVFIVSGRGRESLSNWLSPCENLGIAAEHGYFIRWKSKDEWETCYSPTDTEWRSMVEPVMRSYMEATDGTSIEFKESALVWHHQDADPDFGSCQAKEMLDHLESVLANEPVVVKRGQHIVEVKPQGVSKGLAAEKVIREMVERGEPPEMVMCIGDDRSDEDMFESILSTVTNPELLVQPEVFACTVGRKPSKAKYFLDDEADVLKLLRGLGDSSSSLKPSSSHTQVAFESIV

SEQ ID NO: 3 - DNA, 2604 bp, Arabidopsis thaliana
atggtgtcaagatcttgtgcaaattttctagatttagcatcttgggacttattggactttcctcaaactcaaagagctcttcctcgtgtcatgactgttcctggtatcatctctgagttggatggaggctacagtgatggatcctctgatgttaattcctcaaacagctcccgtgagcggaagattatagtggctaatatgttaccattacaagctaagagagatacagaaactggacaatggtgttttagttgggatgaagattctcttctcttgcaactcagagatgggttttcttcggatacagagtttgtttatataggatcacttaatgctgatattggtattagtgaacaagaagaagtttctcataagcttttgttggatttcaattgtgttcctacgtttttacccaaggagatgcaagagaagttctatcttggtttctgtaaacatcatttgtggccgctatttcactatatgcttcctatgttccctgaccatggtgatcgttttgaccggcgtctttggcaagcgtatgtctctgcaaacaagatattttcagatagggtgatggaagttatcaaccctgaggaagattatgtttggattcatgattatcatctgatggttcttcccacattcttgaggaaacggtttaacaggatcaagcttggatttttccttcacagtccatttccatcatcagaaatttaccgtactttgccagtccgggatgatcttttgagaggattgttgaactgtgatctcattggcttccacacatttgattatgcacgccattttttgtcatgctgcagtagaatgcttgggcttgattatgaatctaagcgtgggcacatcgggcttgattactttggtcgaacggtgtttattaagatccttcctgttggcatccatatggggaggctggaatcggttttgaatcttccgtcgactgcagcgaaaatgaaagagatacaagaacagtttaaggggaaaaagttgattcttggtgttgacgacatggacatctttaaaggcataagcctcaaacttatagccatggaacgtctctttgagacatattggcatatgcgaggaaaacttgtcctgattcagatagtgaacccagcacgggccacgggtaaggatgtggaagaagcaaagaaggagacatattcaactgcaaaaaggatcaatgagcgctatggttctgctggttatcagccagtgatcttgattgatcgtcttgttcctcgttacgagaagactgcttattatgctatggcagactgctgcctggtgaatgcagtaagagacggcatgaacttagttccatataaatatatcatttgcaggcaagggaccccaggaatggataaggccatggggattagccatgactcagcccggacgagcatgcttgttgtctctgagtttatcggctgctcgccttctttgagtggtgcgatcagggtgaacccatgggatgtagatgctgttgcagaagcggtaaacttagccctcactatgggtgagactgaaaagcgattaaggcacgagaaacactatcactatgtgagtactcatgatgtgggttactgggcaaagagctttatgcaggatcttgagagggcatgccgggagcattataataaacgttgttggggtattggttttggcttgagtttcagagttctgtcactgtctccgagttttaggaagctatctatcgatcacattgtctccacgtatagaaatacacagagaagggcaatatttttggactatgatggcactctcgttcctgaaagctccatcatcaaaacccctaatgccgaagtcctgtctgttctgaaatctctgtgtggagatcctaaaaacactgtgtttgttgtcagtggaagaggatgggagtctctgagcgactggctatctccatgtgaaaatcttggaatcgcagctgaacacggatacttcataaggtggagtagcaagaaagaatgggagacttgttattcgtcggctgaggcggaatggaagacgatggtagaaccggtaatgagatcatacatggacgcaaccgatggttctactatagagtacaaagagagtgctttggtttggcatcatcaagacgcagatccagactttggagcctgtcaagcaaaagagcttctagatcatctagagagtgtactcgcaaatgagcctgtagtcgtcaagagaggccaacacattgtagaggtcaaaccacagggagtaagcaaaggtctagcagtggaaaaggtgatacaccaaatggtagaggatggaaacccaccggacatggtgatgtgtataggagatgacagatcagacgaagacatgtttgagagcatattgagcacagtgacaaacccggacctcccaatgccacctgagatctttgcctgcacggtgggaagaaaaccaagcaaagccaaatacttcttagacgatgtctctgatgtattaaagctcctaggaggattagctgctgcaacgagcagctcgaagccagagtatcaacaacaatcctcctcattgcacacgcaagtggcgtttgagagcatcatatga

SEQ ID NO: 4 - protein, 867 aa, Arabidopsis thaliana
MVSRSCANFLDLASWDLLDFPQTQRALPRVMTVPGIISELDGGYSDGSSDVNSSNSSRERKIIVANMLPLQAKRDTETGQWCFSWDEDSLLLQLRDGFSSDTEFVYIGSLNADIGISEQEEVSHKLLLDFNCVPTFLPKEMQEKFYLGFCKHHLWPLFHYMLPMFPDHGDRFDRRLWQAYVSANKIFSDRVMEVINPEEDYVWIHDYHLMVLPTFLRKRFNRIKLGFFLHSPFPSSEIYRTLPVRDDLLRGLLNCDLIGFHTFDYARHFLSCCSRMLGLDYESKRGHIGLDYFGRTVFIKILPVGIHMGRLESVLNLPSTAAKMKEIQEQFKGKKLILGVDDMDIFKGISLKLIAMERLFETYWHMRGKLVLIQIVNPARATGKDVEEAKKETYSTAKRINERYGSAGYQPVILIDRLVPRYEKTAYYAMADCCLVNAVRDGMNLVPYKYIICRQGTPGMDKAMGISHDSARTSMLVVSEFIGCSPSLSGAIRVNPWDVDAVAEAVNLALTMGETEKRLRHEKHYHYVSTHDVGYWAKSFMQDLERACREHYNKRCWGIGFGLSFRVLSLSPSFRKLSIDHIVSTYRNTQRRAIFLDYDGTLVPESSIIKTPNAEVLSVLKSLCGDPKNTVFVVSGRGWESLSDWLSPCENLGIAAEHGYFIRWSSKKEWETCYSSAEAEWKTMVEPVMRSYMDATDGSTIEYKESALVWHHQDADPDFGACQAKELLDHLESVLANEPVVVKRGQHIVEVKPQGVSKGLAVEKVIHQMVEDGNPPDMVMCIGDDRSDEDMFESILSTVTNPDLPMPPEIFACTVGRKPSKAKYFLDDVSDVLKLLGGLAAATSSSKPEYQQQSSSLHTQVAFESII

SEQ ID NO: 5 - DNA, 153 bp, Silene pratensis
atggcttctacactctctaccctctcggtgagcgcatcgttgttgccaaagcaacaaccgatggtcgcctcatcgctaccaaccaacatgggccaagccttgtttggactgaaagccggttctcgtggcagagtgactgcaatggccacatac

SEQ ID NO: 6 - protein, 51 aa, Silene pratensis
MASTLSTLSVSASLLPKQQPMVASSLPTNMGQALFGLKAGSRGRVTAMATY

SEQ ID NO: 7 - DNA, 1401 bp, Zea mays
ccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaatcttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgttttttctataaaaaaagggaaactgcccctcatttacagcaaattgtccgctgcctgtcgtccagatacaatgaacgtacctagtaggaactcttttacacgctcggtcgctcgccgcggatcggagtcccaggaacacgacaccactgtgtaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgctacctccgctttagttctgcatacagccaacccaa

SEQ ID NO: 8 - DNA, 300 bp, Sugarcane bacilliform virus
agatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 9 - DNA, 1421 bp, Sugarcane bacilliform virus
gaagttgaagacaaagaaggtcttaaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

SEQ ID NO: 10 - DNA, 583 bp, Oryza sativa
gtaagatccgatcaccatcttctgaatttctgttcttgatctgtcatgtataataactgtctagtcttggtgttggtgagatggaaattcggtggatctcggaagggatattgttcgtttgctggggttttttttgtgtgttgtgatccgtagagaatttgtgtttatccatgttgttgatcttggtatgtattcatgacatattgacatgcatgtgttgtatgtgtcatatgtgtgcctctccttgggatttgttttggataatagaacatgttatggactcaatagtctgtgaacaaatctttttttagatggtggccaaatctgatgatgatctttcttgagaggaaaaagttcatgatagaaaaatcttttttgagatggtggcttaatgtgatgatgatctttcttgagaggaaaaaaaagattcattataggagattttgatttagctcctttccaccgatattaaatgaggagcatgcatgctgattgctgataaggatctgatttttttatcccctcttctttgaacagacaagaaataggctctgaatttctgattgattatttgtacatgcag

SEQ ID NO: 11 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 12 - DNA, 331 bp, Oryza sativa
gtaagttctggctttcttgcttttggataaattttgcttcctttcttaacttgagcacaagcttgtgttatatgtggtgtggaatcttggttgccatgttgtgaggatttagctagagagtcaagaaagaggaatatatgctttatgtagataggagtaggatctctgggtctttaaacatcaccatgacaagcaaagataagaacaggagagcagttcttgattattatttttcttctcatcaagaaattaagccggagatagacatggcagctgcacgcagtgattcacttcttgatttcttgatttgggttgttgcgtttgtgtccag

SEQ ID NO: 13 - DNA, 709 bp, Agrobacterium tumefaciens
tcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagca

SEQ ID NO: 14 - DNA, 1198 bp, Zea mays
tcccgtgtccgtcaatgtgatactactagcatagtactagtaccatgcatacacacagcaggtcggccgcctggatggatcgatgatgatactacatcatcctgtcatccatccaggcgatctagaaggggcgtggctagctagcaaactgtgaccggtttttctacgccgataataatactttgtcatggtacagacgtacagtactggttatatatatctgtagatttcaactgaaaagctaggatagctagattaattcctgagaaacacagataaaattcgagcttggctatagatgacaaaacggaagacgcatgcattggacgacgtatgcaatgcgagcgcgtctcgtgtcgtcccgtccaagtctggcgatctcacgccacgtgctcaacagctcaaggactgttcgtcaccagcgttaaattcattgaagggatgacgcatttcggcatttgtcattgcttgtagctatatatatatatccaacagatttctctcaagcttttgtatgcgtgaatgtaaagtctagcttatacgacagcacgtgcagatatattaacgtcattattaggtggagagcaagatgcatgatctggtagaaattgtcgaaaacacaagagagagtgaagtgcacacttctggtataggagtgtatacgccgctggttggtgggcaatgcgcgccgcaatattggccaatgaaacctagcaacgcccactcgccacgccccatgaatggcccccgcacgacagcgagccagccagtgcccgcgcgcggcccagccggagtcggcggaacgcgccacgggggacaaggcgcccgagggccgaggcagcgcggcatggcaagcaagccgaagcgggcaagcgacctgcatgcagcccctgcacctcgccctcgtcagtcgtcccagcctcccactggaatccacccaacccgcccttcctctacaaagcacgcgccccgcgactcgcctccgcctacgtgtcggcagcgtccccgccggtcgcccacgtaccccgccccgttctcccacgtgcccctccctctgcgcgcgtccgattggctgacccgcccttcttaagccgcgccagcctcctgtccgggccccaacgccgtgctccgtcgtcgtctccgcccccagagtgatcgagcccactgacctggcccccgagcctcagctcgtgagtcc

SEQ ID NO: 15 - DNA, 856 bp, Zea mays
cgtagcaatgcacgggcatataactagtgcaacttaatacatgtgtgtattaagatgaataagagggtatccaaataaataacttgttcgcttacgtctggatcgaaaggggttggaaacgattaaatctcttcctagtcaaaattaaatagaaggagatttaatcgatttctcccaatccccttcgatccaggtgcaaccgaataagtccttaaatgttgaggaacacgaaacaaccatgcattggcatgtaaagctccaagaattcgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgaca

SEQ ID NO: 16 - DNA, 2589 bp, Arabidopsis thaliana
atggtatcaagatcttattcaaacctcttggatcttgcttctggtaacttccattcgttttctcgagaaaagaagaggtttccaagagtagcaactgtcactggtgtcttatctgagctagatgatgataacaacagcaacagtgtctgctctgatgctccttcttccgtcacacaagatcgaatcatcatcgttggaaaccagcttcctattaaatcacatcggaactctgctggtaaattgagttttagttgggacaatgactcgcttctcttgcagcttaaagatggtatgcgtgaagatatggaagttgtctacattggttgccttaaggaacagattgatacagttgagcaagatgatgtttctcaaaggttgcttgagaatttcaaatgtgttcctgcttatatcccacctgagctattcactaagtactatcatggattctgtaagcaacatctatggcctttgtttcactacatgcttcccttaactcctgatcttggtggtagatttgatcgttctttatggcaagcttatctttcggttaacaagatctttgctgataaagtgatggaagtgattagtcctgatgatgattttgtttgggttcatgactatcacttaatggttttgcctacatttctaaggaagaggtttaatagagtaaagcttggttttttccttcatagcccgttcccttcctctgagatttaccgtactcttccagtgagaaatgagctcttacgtgcactcctcaacgctgatttgattggctttcatacctttgactatgcaagacacttcctttcttgttgtagcaggatgcttggtttatcctatcagtccaaacgtggaaccatagggcttgagtattatggtcgaacggttagtatcaagattcttcctgtggggattcatatcagccagcttcagtcaattttaaacctcccagagactcagaccaaagttgctgagctaagagatcagttcttggatcagaaagttcttctcggtgttgatgacatggacatcttcaaaggaatcagcctcaaactcttggcaatggaacaacttctcacacagcatcccgagaagagaggtcgagttgtacttgtccagattgcaaaccctgcaagaggtcgtgggaaagacgttcaggaggttcagtctgaaactgaagccacggttaaaaggatcaatgaaatgtttggaaggccgggctaccaacccgtggttctgattgatacaccgcttcaattctttgagaggattgcttactatgtcattgcagagtgttgtcttgttacagcggtaagagatggtatgaatcttataccttatgagtacattatctgcaggcaaggtaatccgaaactcaacgagactataggccttgacccttctgctgcaaagaagagtatgcttgttgtctctgagtttattggttgttctccttctttaagcggcgccattagagtaaatccgtggaacattgatgctgtgactgaagcaatggactatgcattgatagtttcagaagcagagaagcaaatgcgtcacgagaagcatcacaaatatgttagcacacatgatgttgcttattgggcgcgtagctttatacaagatcttgaaagggcttgcggggatcatgtgaggaagaggtgttgggggattggattcgggttaggctttagagttgtggcgcttgatccgagttttaaaaagctttcgattgagcacattgtctcagcttataagagaaccaagaaccgagccattttgttggattatgatggcacaatggtgcagccaggttccattaggacaacaccaacccgcgaaacaatcgaaatcttgaacaacctgtctagtgatcccaagaatatcgtgtacctcgtcagtgggaaagacagaaggacactaactgaatggttttcttcatgtgacgatctcggtttgggtgcagagcacgggtattttataaggccaaatgatggaacagactgggaaacgtcgagtttggtatcaggttttgagtggaaacaaatagcagagccagtgatgagactttacactgagactacagatggatcaacaatagagactaaagagactgctctcgtttggaattaccaattcgcagatcctgattttggatcttgtcaagccaaagagcttatggaacacctcgaaagcgtgcttaccaatgatccagtctctgtcaagactggacaacaactcgttgaagttaaaccacagggtgtgaacaaaggtcttgtggcagagaggcttctaacaacgatgcaagaaaaagggaaacttttggatttcattctctgcgtcggtgatgatcggtctgatgaggatatgtttgaggtgataatgagtgctaaagatggtccagctttgtctcctgtggctgagatattcgcttgcaccgttggacaaaagccaagcaaagcaaaatactatttagacgatactgcggagataatcagaatgctggacggtctagccgctaccaacacaactatctctgatcaaactgattcaaccgccactgttccaactaaagatctgttttaa

SEQ ID NO: 17 - protein, 862 aa, Arabidopsis thaliana
MVSRSYSNLLDLASGNFHSFSREKKRFPRVATVTGVLSELDDDNNSNSVCSDAPSSVTQDRIIIVGNQLPIKSHRNSAGKLSFSWDNDSLLLQLKDGMREDMEVVYIGCLKEQIDTVEQDDVSQRLLENFKCVPAYIPPELFTKYYHGFCKQHLWPLFHYMLPLTPDLGGRFDRSLWQAYLSVNKIFADKVMEVISPDDDFVWVHDYHLMVLPTFLRKRFNRVKLGFFLHSPFPSSEIYRTLPVRNELLRALLNADLIGFHTFDYARHFLSCCSRMLGLSYQSKRGTIGLEYYGRTVSIKILPVGIHISQLQSILNLPETQTKVAELRDQFLDQKVLLGVDDMDIFKGISLKLLAMEQLLTQHPEKRGRVVLVQIANPARGRGKDVQEVQSETEATVKRINEMFGRPGYQPVVLIDTPLQFFERIAYYVIAECCLVTAVRDGMNLIPYEYIICRQGNPKLNETIGLDPSAAKKSMLVVSEFIGCSPSLSGAIRVNPWNIDAVTEAMDYALIVSEAEKQMRHEKHHKYVSTHDVAYWARSFIQDLERACGDHVRKRCWGIGFGLGFRVVALDPSFKKLSIEHIVSAYKRTKNRAILLDYDGTMVQPGSIRTTPTRETIEILNNLSSDPKNIVYLVSGKDRRTLTEWFSSCDDLGLGAEHGYFIRPNDGTDWETSSLVSGFEWKQIAEPVMRLYTETTDGSTIETKETALVWNYQFADPDFGSCQAKELMEHLESVLTNDPVSVKTGQQLVEVKPQGVNKGLVAERLLTTMQEKGKLLDFILCVGDDRSDEDMFEVIMSAKDGPALSPVAEIFACTVGQKPSKAKYYLDDTAEIIRMLDGLAATNTTISDQTDSTATVPTKDLF

SEQ ID NO: 18 - DNA, 2589 bp, Arabidopsis thaliana
atgtcgccggaatcttggaaagaccagcttagtctggtttcggctgatgattatcggatcatgggtcgaaatcggatccccaatgccgtcacgaagctttccggtctcgaaaccgacgatcctaacggcggcgcgtgggttacgaaaccgaaacgaatcgtggtttcgaatcagcttcctcttcgtgctcacagagacatttcgtcgaacaagtggtgctttgaattcgacaatgacagtctttacttacaactcaaagatgggtttcctccggagacggaagtcgtctacgtcggatctttaaacgccgacgtcttaccttcagagcaagaggacgtctctcagttcttgcttgagaagtttcagtgtgttcctactttcttacctagtgacttgctcaacaagtattaccatggtttctgtaaacactatctctggcccatttttcactatcttcttcctatgacgcaagctcaaggctctctctttgatcgttcgaattggagagcgtacacgactgttaacaagatcttcgctgataagatcttcgaagtgctaaacccggatgatgattacgtctggattcatgattatcacctcatgattttgcccacttttttgaggaacaggtttcatcggataaagcttgggattttcctccatagtccctttccttcgtcggagatttaccgtactcttcctgtgagagacgagattctcaaagggtttctgaattgcgatttggttggtttccacacgtttgattacgctaggcatttcttgtcttgttgtagtaggatgcttggtcttgattacgaatctaaaagaggctacattggtcttgaatattttggaagaacggtgagcatcaagatattgcccgttgggattcatatggggcagattgaatcgataaaggcttcggagaaaactgcagagaaagtgaagagattgagagaaaggttcaaggggaacattgtgatgttaggtgtggatgatttggatatgttcaaaggtattagcttgaagttttgggcgatgggtcagcttcttgaacagaacgaagagcttcgtgggaaagtggttctcgtgcagattactaatcctgctcgtagttcaggtaaggatgttcaagatgtagagaaacagataaatttgattgctgatgagatcaattctaaatttgggagacctggtggttataagcctattgtgtttatcaatggacctgttagtactttggataaagttgcttattacgcgatctcggagtgtgttgtcgtgaatgctgtgagagatgggatgaatttggtgccttataagtacacagtgactcggcaagggagccctgctttggatgcagctttaggttttggggaggatgatgttaggaagagtgtgattattgtttctgagttcatcggttgttctccatctctgagtggtgcgatccgtgttaatccgtggaacatcgatgcagtcactaacgccatgagctctgcaatgacgatgtccgacaaagagaaaaatctgcgccaccagaagcatcataagtacataagctctcacaatgttgcctattgggcgcggagttatgaccaagatcttcaaagggcgtgcaaagatcattacaacaagagattctggggagtcggattcggtctttttttcaaggttgttgcgttagatccgaatttcagaaggctctgtggtgaaaccatagtccccgcgtataggagatcaagcagtaggttgatcctattggactatgatgggacaatgatggatcaggatactctggataaaaggccaagtgatgatcttatctcgcttctcaatcgcttatgtgacgaccccagcaatctagtctttattgttagtggtcgaggtaaggaccctctcagcaaatggtttgactcttgcccaaatcttggtatctcagctgaacatggttatttcactagatggaactcaaattccccttgggaaacaagtgaattgcctgcggatttaagctggaagaaaatagctaaaccagtgatgaatcactatatggaagcgactgatggatcattcatagaggagaaagagagtgctatggtgtggcaccaccaagaagctgaccattcatttggttcttggcaagctaaggagcttcttgatcatctagagagtgttctcaccaatgagcctgttgttgtcaagagaggccagcacatagtagaagttaaacctcagggagtaagcaaaggaaaggtggtggagcatttgatagcaacgatgaggaacaccaaagggaagagaccggactttttgttgtgcataggtgatgaccggtctgatgaagacatgtttgatagcatagtgaagcaccaagatgtttcctctattggtctcgaagaggtctttgcgtgcacagttggtcagaaaccgagcaaggccaagtactatctcgatgataccccaagtgttatcaagatgcttgaatggttggcctcagcttcagatggatcaaagcatgagcaacagaagaaacagagcaagttcacttttcaacagcctatgggacaatgtcgaaagaaagcatag

SEQ ID NO: 19 - protein, 862 aa, Arabidopsis thaliana
MSPESWKDQLSLVSADDYRIMGRNRIPNAVTKLSGLETDDPNGGAWVTKPKRIVVSNQLPLRAHRDISSNKWCFEFDNDSLYLQLKDGFPPETEVVYVGSLNADVLPSEQEDVSQFLLEKFQCVPTFLPSDLLNKYYHGFCKHYLWPIFHYLLPMTQAQGSLFDRSNWRAYTTVNKIFADKIFEVLNPDDDYVWIHDYHLMILPTFLRNRFHRIKLGIFLHSPFPSSEIYRTLPVRDEILKGFLNCDLVGFHTFDYARHFLSCCSRMLGLDYESKRGYIGLEYFGRTVSIKILPVGIHMGQIESIKASEKTAEKVKRLRERFKGNIVMLGVDDLDMFKGISLKFWAMGQLLEQNEELRGKVVLVQITNPARSSGKDVQDVEKQINLIADEINSKFGRPGGYKPIVFINGPVSTLDKVAYYAISECVVVNAVRDGMNLVPYKYTVTRQGSPALDAALGFGEDDVRKSVIIVSEFIGCSPSLSGAIRVNPWNIDAVTNAMSSAMTMSDKEKNLRHQKHHKYISSHNVAYWARSYDQDLQRACKDHYNKRFWGVGFGLFFKVVALDPNFRRLCGETIVPAYRRSSSRLILLDYDGTMMDQDTLDKRPSDDLISLLNRLCDDPSNLVFIVSGRGKDPLSKWFDSCPNLGISAEHGYFTRWNSNSPWETSELPADLSWKKIAKPVMNHYMEATDGSFIEEKESAMVWHHQEADHSFGSWQAKELLDHLESVLTNEPVVVKRGQHIVEVKPQGVSKGKVVEHLIATMRNTKGKRPDFLLCIGDDRSDEDMFDSIVKHQDVSSIGLEEVFACTVGQKPSKAKYYLDDTPSVIKMLEWLASASDGSKHEQQKKQSKFTFQQPMGQCRKKA

SEQ ID NO: 20 - DNA, 2586 bp, Glycine max
atggcttcaagatcatatgctaatctctttgacttagctagtggagactttcttgattttccttgcaccccaagagctcttccaagggttatgactgttcctggaattatttcggacctggatggttatggttgtaatgatggggattcagatgttagttcttctggatgtagggagcggaaaatcattgtggcaaacatgttgccagtgcaggctaaaagagatatagaaactgctaaatgggttttcagttgggatgaggattcaattttgttacaattaaaagatggtttttctgctgatagtgaggtaatctatgtgggttctctcaaggttgaaatagatgcctgtgagcaggatgcagttgctcagagattgctagatgaatttaattgtgtacctacctttcttccccatgatctccaaaaaaggttctaccttggattttgtaagcagcaactttggcctctatttcattatatgctacctatatgcccagatcacggtgatcgctttgaccgtatactttggcaggcttatgtttctgcaaacaaaatatttgcagacaaggtcatggaagtaattaatcctgatgatgattttgtttgggttcatgattatcacttaatggttttgcctactttcttgaggaagcgatataatcgggttaaacttgggttctttctgcatagtcctttcccttcatctgaaatctaccgaactttaccagtaagggatgaaattttgaggggattgttgaactctgatttaattggctttcatacatttgattatgctcgccactttctttcttgctgcagtagaatgctaggtctggactatgaatctaagcgaggacatatagggcttgattactttggccgcactatatttattaaaattttgcctgtaggcattcacatgggtaggcttgaatctgtgttaaatctttcttctacatctgctaaactaaaagaggttcaggaagagtttaaggataagaaagtaattcttggtattgatgacatggatatttttaagggcattagtctgaaacttctagctgtggagcatctgctgcagcagaatccagatttgcagggcaaagttgtcctagttcaaattgtaaatcctgcaaggggctcggggaaggatgttcaggaagcaaagaacgaaacatatttaattgcccagagaatcaacgatacatatagctcaaataattatcagccagtcattctcattgaccgccctgttcctcgctttgagaagagtgcctattatgctgtagctgaatgttgcattgttaatgctgtaagggatggtatgaacttagtcccatacaaatatatcgtctgcagacagggaactgcacaactagatgaagcattggatagaaaaagtgattctcctcgtacaagcatgcttgtggtgtctgagttcattggttgttcaccttctcttagtggggcaataagggtcaatccctgggacatagatgccgtagccgatgctatgtatgcagcccttacaatgagtgtttcagagaagcagttgcgccatgagaaacactatcggtatgtgagttctcatgatgttgcatattgggcgcacagctttatgctggatttggagagagcctgcaaagatcattacaccaaaagatgctggggatttggtttgggcttggggttcagagttgtttctctttctcatggtttcaggaagctgtcaattgaccatattgtttcagcatacaagagaaccaatagaagggccatctttcttgattatgatggtactgttgtacctcaatcttccataagtaaaacccccagccctgaagtcatctctgtcttaaatgctctgtgtaacaatcccaagaatattgtgttcattgttagtgggagggggagggattcactgagtgaatggtttacttcatgccaaatgcttggacttgcagcagaacatgggtactttttaaggtggaacaaagattcagaatgggaagcaagtcacttatctgcggaccttgattggaaaaagatggtggaacctgtgatgcagttgtatacagaagcaactgatggttctaatattgaagttaaggagagtgctttggtgtggcatcatcaagatgcagaccctgattttggttcttgccaagccaaagaattgttggatcacttggaaagtgtgcttgctaatgaaccagcagctgttacgagaggtcagcatattgttgaagttaagccacagggaataagcaaggggttggtagctgaacaggttcttatgaccatggttaatggcggcaatccaccagattttgtgctgtgcattggagatgataggtccgatgaggacatgtttgagagcattttgaggacagtttcgtgcccatcattaccatcagctccagagatctttgcctgcactgtgggtaggaagcctagcaaggccaagtattttcttgatgatgcttctgatgttgtgaagttgcttcagggccttgctgcttcatccaatccaaaacccaggcatcttgctcattctcaagtctcttttgagagcacagtttga

SEQ ID NO: 21 - protein, 861 aa, Glycine max
MASRSYANLFDLASGDFLDFPCTPRALPRVMTVPGIISDLDGYGCNDGDSDVSSSGCRERKIIVANMLPVQAKRDIETAKWVFSWDEDSILLQLKDGFSADSEVIYVGSLKVEIDACEQDAVAQRLLDEFNCVPTFLPHDLQKRFYLGFCKQQLWPLFHYMLPICPDHGDRFDRILWQAYVSANKIFADKVMEVINPDDDFVWVHDYHLMVLPTFLRKRYNRVKLGFFLHSPFPSSEIYRTLPVRDEILRGLLNSDLIGFHTFDYARHFLSCCSRMLGLDYESKRGHIGLDYFGRTIFIKILPVGIHMGRLESVLNLSSTSAKLKEVQEEFKDKKVILGIDDMDIFKGISLKLLAVEHLLQQNPDLQGKVVLVQIVNPARGSGKDVQEAKNETYLIAQRINDTYSSNNYQPVILIDRPVPRFEKSAYYAVAECCIVNAVRDGMNLVPYKYIVCRQGTAQLDEALDRKSDSPRTSMLVVSEFIGCSPSLSGAIRVNPWDIDAVADAMYAALTMSVSEKQLRHEKHYRYVSSHDVAYWAHSFMLDLERACKDHYTKRCWGFGLGLGFRVVSLSHGFRKLSIDHIVSAYKRTNRRAIFLDYDGTVVPQSSISKTPSPEVISVLNALCNNPKNIVFIVSGRGRDSLSEWFTSCQMLGLAAEHGYFLRWNKDSEWEASHLSADLDWKKMVEPVMQLYTEATDGSNIEVKESALVWHHQDADPDFGSCQAKELLDHLESVLANEPAAVTRGQHIVEVKPQGISKGLVAEQVLMTMVNGGNPPDFVLCIGDDRSDEDMFESILRTVSCPSLPSAPEIFACTVGRKPSKAKYFLDDASDVVKLLQGLAASSNPKPRHLAHSQVSFESTV

SEQ ID NO: 22 - DNA, 2475 bp, Oryza sativa
atgccctccctccccaactccggcgacgagggcggcgccccgcctccgactccgccgccgccgggggcgcgccgcgtggtggtcgcccaccgcctccccctccgcgcggatcccaatccgggcgcgccgcacgggttcgacttctccctcgacccgcacgcgctcccgctccagctctcccatggcgtcccccgccccgtcgtcttcgtcggcgtgctcccctccgccgtcgccgaggccgtccaggcgtccgacgagctcgcggccgatctcctcgcgcggttctcatgctacctggtgttcctccccgccaagctccacgccgacttctacgacggcttctgcaagcactacatgtggccgcatctccactatctcctcccgctcgcgccctcctacggcaggggcggcggcctccccttcaacggcgacctctaccgcgccttcctcaccgtcaacacccacttcgccgagcgcgtgttcgagctcctcaaccccgacgaggacctggtgttcgtccacgactaccacctctgggcgttccccaccttcctccgccacaaatccccgcgcgcccgcataggtttcttcctccactctcccttcccctcctccgagctcttccgcgccatccccgtccgcgaggacctcctccgcgccctcctcaacgccgatctcgtgggcttccacaccttcgattacgcgcgccacttcctctccgcgtgctccagggtcctcggcctctccaaccgctcgcgccgcggctacatcgggatcgagtacttcggccgcacggtggtcgtcaagatcctctccgtcggcatcgacatgggccagctccgcgcggttctgccgttgccggagacggtcgccaaggccaacgagattgctgacaagtacaggggacgacagctgatgctcggcgtggacgacatggatttgttcaaggggattgggctcaagctcttggccatggagaggctgctggagtcgcgggcggacttgcgtggccaggtggtcctcgtgcagatcaacaacccggcgcggagccttggccgcgacgtcgacgaggtccgcgcggaggtgctggcgatccgtgaccggatcaatgcccggttcggctgggcggggtacgagccggtggttgtgatcgacggcgccatgccgatgcacgacaaggtggcgttctacacgtccgcggacatctgcatcgtgaatgccgtgcgcgacgggctgaacaggataccgtacttctacaccgtgtgccggcaggagggcccggttcccaccgctcctgccgggaagccgaggcagagcgccatcatcgtgtcagagtttgtcgggtgctcgccgtcgctgagcggcgcgatccgcgtcaacccctggaacgtggacgacgtcgcggacgccatgaacacggcgctgaggatgagcgacggcgagaagcagctgcgccaggagaagcactacaggtacgtgagcacgcacgacgtcgtctactgggcgcagtcgttcgaccaggacctgcagaaggcctgcaaggacaactcgtccatggtgatcctcaacttcggcctcggcatgggcttccgcgtcgtcgcgctcggccccagcttcaagaaactctcacccgagctcattgaccaagcataccgccagactggcaacaggctcatcttactggactacgatggcacagtgatgccacaggggctgatcaacaaggcgcccagtgaggaagtgatccgtactctgaatgaactgtgctctgatccgatgaacaccgttttcgtcgtcagcgggcggggcaaggatgaactggctgaatggtttgcaccatgcgacgagaagctggggatctctgcagagcacggctacttcacaaggtggagcagggattctccctgggagtcatgcaagttggtgacacattttaattggaagaacatcgcagggcctgtaatgaagcactacagtgatgcaaccgatgggtcatacatcgaggttaaagaaacatcactagtgtggcactatgaggaagccgatccggattttggatcatgccaggccaaagagctccaggaccacctgcagaatgtgcttgcgaacgagccagtctttgtgaagagcggccatcagattgtggaagttaatcctcagggtgtgggcaaaggagtcgccgtgcgtaatctcatttcaaccatgggaaatcgtggcagcttgccagatttcatcctctgcgtcggcgatgaccggtcggatgaagacatgttcgaggctatgatcagcccttcgcctgcgttcccggagactgcacagatcttcccctgcactgttggcaacaagccgagcttggccaagtactacctggatgacccggctgatgttgtgaagatgctccagggcctgacggactcgccgacccagcagcaaccgcggccccccgtctcgttcgaaaactcgctagatgattga

SEQ ID NO: 23 - protein, 824 aa, Oryza sativa
MPSLPNSGDEGGAPPPTPPPPGARRVVVAHRLPLRADPNPGAPHGFDFSLDPHALPLQLSHGVPRPVVFVGVLPSAVAEAVQASDELAADLLARFSCYLVFLPAKLHADFYDGFCKHYMWPHLHYLLPLAPSYGRGGGLPFNGDLYRAFLTVNTHFAERVFELLNPDEDLVFVHDYHLWAFPTFLRHKSPRARIGFFLHSPFPSSELFRAIPVREDLLRALLNADLVGFHTFDYARHFLSACSRVLGLSNRSRRGYIGIEYFGRTVVVKILSVGIDMGQLRAVLPLPETVAKANEIADKYRGRQLMLGVDDMDLFKGIGLKLLAMERLLESRADLRGQVVLVQINNPARSLGRDVDEVRAEVLAIRDRINARFGWAGYEPVVVIDGAMPMHDKVAFYTSADICIVNAVRDGLNRIPYFYTVCRQEGPVPTAPAGKPRQSAIIVSEFVGCSPSLSGAIRVNPWNVDDVADAMNTALRMSDGEKQLRQEKHYRYVSTHDVVYWAQSFDQDLQKACKDNSSMVILNFGLGMGFRVVALGPSFKKLSPELIDQAYRQTGNRLILLDYDGTVMPQGLINKAPSEEVIRTLNELCSDPMNTVFVVSGRGKDELAEWFAPCDEKLGISAEHGYFTRWSRDSPWESCKLVTHFNWKNIAGPVMKHYSDATDGSYIEVKETSLVWHYEEADPDFGSCQAKELQDHLQNVLANEPVFVKSGHQIVEVNPQGVGKGVAVRNLISTMGNRGSLPDFILCVGDDRSDEDMFEAMISPSPAFPETAQIFPCTVGNKPSLAKYYLDDPADVVKMLQGLTDSPTQQQPRPPVSFENSLDD

SEQ ID NO: 24 - DNA, 2637 bp, Oryza sativa
atgttctcgcgatcctacaccaacctggtcgatctcgccaacggcaacctctccgccctggactatggtggcggagggggagggggcggcggcggcaacggggccgggggccggccgccgcgggcgaggcggatgcagcggacgatgacgacgcccgggacgctggcggagctcgacgaggagcgggccgggagcgtcacctccgacgtgccctcgtcgctcgccagcgaccgcctcatcgtcgtcgccaacaccctccccgtgcgctgcgagcgccgccccgacgggcgcgggtggagcttctgctgggacgaggactccctcctcctccacctccgcgacggcctccccgatgacatggaggtcctctacgtcggctccctccgcgccgacgtgccgtccgccgagcaggacgacgtcgcgcaggcgctcctcgaccggttccgctgcgtcccggctttcctccccaaggacgtcttggacagattctaccatggcttctgcaagcagacgctgtggccgctcttccactacatgctccccttcacctccgaccatggcggccgcttcgatcgctcccagtgggaggcatacgtcctcgccaacaagctcttctcccagcgcgtcatcgaggtcctcaaccccgaggatgactacatctggatccacgattaccacctcctcgccctcccgtccttccttcgccgtcggttcaacaggctccgcatcggtttcttcctgcacagcccgttcccttcgtcggaactctaccgttccctccctgttcgcgacgagatcctcaaatcactgctaaactgcgatctgattgggttccacacctttgattacgcgcggcatttcctgtcctgctgcagccggatgctggggatcgagtaccagtcgaagaggggatatatcggtctcgattactttggccgcactgttgggataaagatcatgcctgttgggattaacatgacgcagctgcagacgcagatccggctgcctgatcttgagtggcgtgtcgccgaactccggaagcagtttgatgggaagactgtcatgctcggtgtggatgatatggacatatttaaggggattaatctgaaagttcttgcgtttgagcagatgctgaggacacacccaaaatggcagcgcaaggcagtgttggtgcagattgcaaacccaaggggtggtggtgggaaggaccttgaagagatacaggctgagattgatgagagttgcaggaggataaatgcacaattttcacggccaggatatgttcctgtggtgattatcaatagagccctttcaagtgtggagaggatggcttattataccgtggcagagtgtgtcgttgtaactgcagtgagggatgggatgaacctcacaccatatgagtatattgtctgtagacagggatttccagatttggatggttctggggatgatgggccaaggagaaagagtatgttagttgtgtccgaattcattggttgctcaccatcacttagtggagcaattcgggtaaacccttggaacattgatacaacagcagaggcaatgaacgagtcgattgctttatcagagaacgagaagcaactgcggcatgagaagcattacagatatgtcagctcacatgatgttgcctattggtccaagagctatattcatgatttggagagaagctgcagggaccattttaggaggaggtgctggggtattggactaggatttggatttagagtagttgctcttgaccgcaacttcaaaaagcttactgtggattctatcgttactgattacaagaattctaagagcagggttatactgctagactacgatggaacgctagtaccacaaactacaatcaacaggactccaaatgaaagtgttgttaaaatcatgaatgctctttgtgacgataagaagaatgttgtttttattgttagtggacgaggaagggatagccttgagaaatggttttccccttgccaggatcttggcattgctgccgaacatggctactttatgaggtggaccagagatgagcaatggcaattgaataaccagtgctcagaatttggatggatgcagatggccaagccagttatgaacctgtatacagaagcaaccgatggatcatatattgaaaccaaagagagtgctttggtctggcaccaccaagatgctgaccctggttttggatcttcacaagctaaagagatgctagatcatttggaaagtgttcttgctaatgagcctgtctgtgtaaagagtggccaacagattgtggaagtgaaaccgcagggtgtcagcaaaggatttgttgctgagaagatcctatcaacgctgacagagaacaagagacaggcagattttgttctctgcataggcgatgatagatcagacgaggatatgtttgaaggaattgctgatatcatgagaaggagcatagttgatccccaaacatcattatatgcgtgcacagtcggccagaagccaagcaaggctaagtactatttggacgatactaatgatgttttgaacatgcttgaggcgcttgcagatgcatcagaggagactgattcacaggaagatgcagaagagataacatctatcccggacccggaatag

SEQ ID NO: 25 - protein, 878 aa, Oryza sativa
MFSRSYTNLVDLANGNLSALDYGGGGGGGGGGNGAGGRPPRARRMQRTMTTPGTLAELDEERAGSVTSDVPSSLASDRLIVVANTLPVRCERRPDGRGWSFCWDEDSLLLHLRDGLPDDMEVLYVGSLRADVPSAEQDDVAQALLDRFRCVPAFLPKDVLDRFYHGFCKQTLWPLFHYMLPFTSDHGGRFDRSQWEAYVLANKLFSQRVIEVLNPEDDYIWIHDYHLLALPSFLRRRFNRLRIGFFLHSPFPSSELYRSLPVRDEILKSLLNCDLIGFHTFDYARHFLSCCSRMLGIEYQSKRGYIGLDYFGRTVGIKIMPVGINMTQLQTQIRLPDLEWRVAELRKQFDGKTVMLGVDDMDIFKGINLKVLAFEQMLRTHPKWQRKAVLVQIANPRGGGGKDLEEIQAEIDESCRRINAQFSRPGYVPVVIINRALSSVERMAYYTVAECVVVTAVRDGMNLTPYEYIVCRQGFPDLDGSGDDGPRRKSMLVVSEFIGCSPSLSGAIRVNPWNIDTTAEAMNESIALSENEKQLRHEKHYRYVSSHDVAYWSKSYIHDLERSCRDHFRRRCWGIGLGFGFRVVALDRNFKKLTVDSIVTDYKNSKSRVILLDYDGTLVPQTTINRTPNESVVKIMNALCDDKKNVVFIVSGRGRDSLEKWFSPCQDLGIAAEHGYFMRWTRDEQWQLNNQCSEFGWMQMAKPVMNLYTEATDGSYIETKESALVWHHQDADPGFGSSQAKEMLDHLESVLANEPVCVKSGQQIVEVKPQGVSKGFVAEKILSTLTENKRQADFVLCIGDDRSDEDMFEGIADIMRRSIVDPQTSLYACTVGQKPSKAKYYLDDTNDVLNMLEALADASEETDSQEDAEEITSIPDPE

SEQ ID NO: 26 - DNA, 2583 bp, Oryza sativa
atggtttctcggtcctactccaacctgctggacctggccaccggcgcggcggaccaggcgccggcgccggcggcgctcggcgcgctccggcggcggctgccgcgggtggtgaccaccgcggggctcatcgacgactccccgctgtccccctcgacgccgtccccgtcgccgcggccgcgcaccatcgtggtcgccaaccacctccctatccgggctcaccgcccggcgtcgccgtcggagccgtggaccttctcctgggacgaggactccctcctccgccacctccagcactcgtcctcctcccccgccatggagttcatctacatcggctgcctccgcgacgacatcccgctggccgaccaggacgccgtcgcgcaggcgctcctcgagtcgtacaactgcgtgccggcgttcctgccccccgacatcgccgagcgctactaccatggcttctgcaagcagcatctgtggccgctgttccactacatgctgccgctctcccccgacctcggcggccgcttcgaccgcgcgctgtggcagtcgtacgtgtcggcgaacaagatcttcgcggacaaggtgctcgaggtgatcaacccggacgacgacttcgtgtgggtgcacgactaccacctcatggtgctcccaaccttcctccgcaagcgcttcaaccgcatcaagctcggcttcttcctccactcgccgttcccctcgtcggagatctacaagacgctccccgtccgggaggagctcctgcgcgcgctgctcaactccgacctcatcggcttccacaccttcgactacgcgcgccacttcctctcctgctgcggccggatgctggggctctcctacgagtccaagcgtggccacatctgcttggagtactacggccggacggtgagcatcaagatcctcccggtgggggtgaacatggggcagctcaagacggtgctcgcgttgccggagacggaggcgaaggtggcagagctgatggcgacttactccgggaaggggagggtcgtcatgctgggcgtcgacgacatggacatcttcaaggggatcagcctcaagctgctcgccatggaggagctgctgcggcagcaccccgagtggcgcggcaagctggtgctcgtccaggtcgcgaacccggcgcgcggccgcggcaaggacgtcgacgaggtgaagggggagacgtacgccatggtgcggcggatcaacgaggcgtacggcgcgcccgggtacgagccggtggtgctcatcgacgagccgctccagttctacgagcgcgtggcgtactacgtcgtcgccgaggtgtgcctggtgaccgcggtccgcgacggcatgaacctgatcccctacgagtacatcgtgtccaggcagggcaacgaggcgctcgacaggatgctgcagccgagcaagccggaggagaagaagagcatgctggtggtgtccgagttcatcgggtgctcgccgtcgctgagcggcgcggtgagggtgaacccgtggaacatcgaggccgtggcggacgccatggagagcgcgctcgtgctgccggagaaggagaagcggatgcgccacgacaagcactaccgctacgtggacacccacgacgtgggctactgggcgaccagcttcctgcaggacctcgagaggacgtgcaaggatcacgcgcagcggcggtgctggggcatcggcttcgggctgcggttcagggtggtgtcgcttgacctcagcttcaggaagctcgccatggagcacattgtcatggcgtaccggagggcgaagacgcgcgccatcctgctcgactacgacggcacgctcatgccgcaggcgatcaacaagagcccgagcgccaattccgtcgaaacgctgaccagcttgtgcagggacaagagcaacaaggttttcctctgcagcgggttcgagaagggaacactccatgactggttcccctgcgagaaccttggcttggcggctgagcacggttacttcctgaggtcatcgagggatgcagagtgggagatttccattccccccgcggactgcagctggaagcagatcgcggagccggtgatgtgcctgtacagggagaccacggacggctcgatcatcgagaacagggagacggtgctcgtctggaactacgaggacgcagaccctgacttcggttcatgccaagccaaggagctcgtcgaccacctcgagagcgtgctcgccaacgagcccgtctcggtgaagagcaccggccattccgttgaggtcaagccacagggcgtgagcaagggcctggtggcgcggcggctgctggcgagcatgcaggagaggggcatgtgcaccgacttcgtgctgtgcatcggggacgaccgctccgacgaggaaatgttccagatgatcacaagctccacctgcggcgagtcgctggcggccacggcggaggtcttcgcctgcaccgtcggccgcaagccgagcaaggccaagtactacctcgacgacacggcggaggtcgtcaggctgatgcagggcttggcctccgtctccaacgagctggctcgggcggcgagccccccggaggacgacgacgaatga

SEQ ID NO: 27 - protein, 860 aa, Oryza sativa
MVSRSYSNLLDLATGAADQAPAPAALGALRRRLPRVVTTAGLIDDSPLSPSTPSPSPRPRTIVVANHLPIRAHRPASPSEPWTFSWDEDSLLRHLQHSSSSPAMEFIYIGCLRDDIPLADQDAVAQALLESYNCVPAFLPPDIAERYYHGFCKQHLWPLFHYMLPLSPDLGGRFDRALWQSYVSANKIFADKVLEVINPDDDFVWVHDYHLMVLPTFLRKRFNRIKLGFFLHSPFPSSEIYKTLPVREELLRALLNSDLIGFHTFDYARHFLSCCGRMLGLSYESKRGHICLEYYGRTVSIKILPVGVNMGQLKTVLALPETEAKVAELMATYSGKGRVVMLGVDDMDIFKGISLKLLAMEELLRQHPEWRGKLVLVQVANPARGRGKDVDEVKGETYAMVRRINEAYGAPGYEPVVLIDEPLQFYERVAYYVVAEVCLVTAVRDGMNLIPYEYIVSRQGNEALDRMLQPSKPEEKKSMLVVSEFIGCSPSLSGAVRVNPWNIEAVADAMESALVLPEKEKRMRHDKHYRYVDTHDVGYWATSFLQDLERTCKDHAQRRCWGIGFGLRFRVVSLDLSFRKLAMEHIVMAYRRAKTRAILLDYDGTLMPQAINKSPSANSVETLTSLCRDKSNKVFLCSGFEKGTLHDWFPCENLGLAAEHGYFLRSSRDAEWEISIPPADCSWKQIAEPVMCLYRETTDGSIIENRETVLVWNYEDADPDFGSCQAKELVDHLESVLANEPVSVKSTGHSVEVKPQGVSKGLVARRLLASMQERGMCTDFVLCIGDDRSDEEMFQMITSSTCGESLAATAEVFACTVGRKPSKAKYYLDDTAEVVRLMQGLASVSNELARAASPPEDDDE

SEQ ID NO: 28 - DNA, 2574 bp, Solanum tuberosum
atgatgtctagatcgtataccaatcttttggatttggcatctgggaattttccagtaatgggaagagagagggataggcgacggatgtcgcgggtaatgacagttcctgggagtatatgtgaactggatgatgaccaggctgttagtgtttcttctgataatcaatcttcacttgctggtgatcggatgattgttgtggcaaatcagttgccattgaaagcgaaaaggagaccggataataagggctggagttttagttggaatgaggattctttgcttttgagacttaaggatggtttacctgaagatatggaagtattgtttgttgggtctttatctgttgatgttgatccaattgaacaggatgatgtttctagctatcttttggataaattcagatgtgtgccaacgtttcttccacctaatatcgtggaaaaatactatgagggattctgcaagaggcatttgtggccactttttcactacatgttaccattttcacctgaccatggaggccgctttgatcgttctatgtgggaagcatatgtttctgccaacaagatgttttcacagaaagtggttgaggtgcttaatcctgaggatgattttgtttggattcatgattatcatttgatggtgttgcccacgttcttgagaaggcggttcaatcgattgaggattgggtttttccttcacagtccatttccttcatctgagatttacaggacacttcctgttagagaggaaatacttaaggctcttctatgttctgaccttgttggattccacactttcgactatgctcgacatttcctttcgtgttgcagtcgaatgttgggtttagagtaccagtctaaaagaggttatataggattggaatattatggaaggacagtaggtatcaagattatgccagtagggatacatatgggtcatattgagtctatgaagaaaattgcagataaagagctgaagtttaaggagctcaaacaacaatttgaaggaaaaactgttctgcttggagttgatgacttggatattttcaaaggtatcaacttaaagcttctagcaatggagcacatgctcaaacagcaccccagttggcaagggcaggctgtgcttgttcagattgccaatcctatgaggggtaaaggaatagatttagaggaaatacaggctgagatacaggaaagctgcaagaggattaataagcaatttggcaagcctggatatgagcctatagtttatattgataggtccgtgtccagtagcgagcgtatggcttattatagtgttgctgaatgtgttgttgtcacagctgttagggatgggatgaacctgactccatatgaatacatcgtttgtcgacagggtgtatcgggtgcagaaacagattcaggtgtaggtgaacctgacaagagcatgctagttgtgtcagaattcattgggtgttctccttccttaagtggggcaatccgtattaatccatggaatgttgaggcaactgctgaggcaatgaatgaggctgtgtcaatggctgaacaagagaaacagctacgacatgagaagcattaccgttatgtcagcacccacgatgttgcttattggtcgagaagtttcttgcaagatatggagagaacttgtgctgatcactttaggaaaagatgctatggcattggtttaggctttgggtttagagttgtttccctagatcccaacttcaggaagctgtcaattgatgatattgtgaatgcttatatcaagtctaagagcagggccatattcctggactatgacggaactgtgatgccgcagaattctatcattaagtctcctagtgctaatgttatctccatcctgaataaactttctggtgatccaaacaacaccgtcttcattgttagtggaagagggagggaaagcctaaccaagtggttttctccttgtagaaaactaggacttgcagcagaacatggctactttttgagatgggaacgagaacagaaatgggaagtatgcagtcagacctctgattttgggtggatgcaacttgctgaacccgtgatgcaatcctatacagacgctacagatggttcttgcatagaaagaaaggaaagtgctatagtgtggcagtatcgtgatgcggattctggatttgggttttctcaggcaaaggagatgcttgatcatctggagagtgttttagcgaatgaaccggttgccgtgaaaagcggtcagcacattgtggaagtgaagcctcagggggtcaccaaaggtttagttgcagaaaaagtctttacatctttagcagtgaaaggaaaactggcggattttgtgctttgcattggtgatgatagatcagatgaagatatgtttgaaatcattggcgatgctttgtccagaaatattatttcatatgatgccaaggtatttgcttgcacagttggacaaaaacctagcaaagcaaagtattacttggatgacacatctgaggtggtgcttatgctagactcccttgctgatgccactgatactccagtgacttccgatgatgaacctgtggactccgactga

SEQ ID NO: 29 - protein, 857 aa, Solanum tuberosum
MMSRSYTNLLDLASGNFPVMGRERDRRRMSRVMTVPGSICELDDDQAVSVSSDNQSSLAGDRMIVVANQLPLKAKRRPDNKGWSFSWNEDSLLLRLKDGLPEDMEVLFVGSLSVDVDPIEQDDVSSYLLDKFRCVPTFLPPNIVEKYYEGFCKRHLWPLFHYMLPFSPDHGGRFDRSMWEAYVSANKMFSQKVVEVLNPEDDFVWIHDYHLMVLPTFLRRRFNRLRIGFFLHSPFPSSEIYRTLPVREEILKALLCSDLVGFHTFDYARHFLSCCSRMLGLEYQSKRGYIGLEYYGRTVGIKIMPVGIHMGHIESMKKIADKELKFKELKQQFEGKTVLLGVDDLDIFKGINLKLLAMEHMLKQHPSWQGQAVLVQIANPMRGKGIDLEEIQAEIQESCKRINKQFGKPGYEPIVYIDRSVSSSERMAYYSVAECVVVTAVRDGMNLTPYEYIVCRQGVSGAETDSGVGEPDKSMLVVSEFIGCSPSLSGAIRINPWNVEATAEAMNEAVSMAEQEKQLRHEKHYRYVSTHDVAYWSRSFLQDMERTCADHFRKRCYGIGLGFGFRVVSLDPNFRKLSIDDIVNAYIKSKSRAIFLDYDGTVMPQNSIIKSPSANVISILNKLSGDPNNTVFIVSGRGRESLTKWFSPCRKLGLAAEHGYFLRWEREQKWEVCSQTSDFGWMQLAEPVMQSYTDATDGSCIERKESAIVWQYRDADSGFGFSQAKEMLDHLESVLANEPVAVKSGQHIVEVKPQGVTKGLVAEKVFTSLAVKGKLADFVLCIGDDRSDEDMFEIIGDALSRNIISYDAKVFACTVGQKPSKAKYYLDDTSEVVLMLDSLADATDTPVTSDDEPVDSD

SEQ ID NO: 30 - DNA, 2181 bp, Crocosphaera watsonii
atgtcaaaaactatcattgtttccaatagacttccggtaaagatcgaaagaaaccaagccggagaatttgagtataaaaccagtgagggaggtctagctacagggcttgggtcggtttataaagaaggtgataatatatgggttgggtggccaggattagctgtcaacaaaactgaagacaaagaggaaatatgctctagattgaaagagtcaaatatgagtcctgtattcctcactaaaaatgaaatagaagaatactatgaaggctttagtaatgagaccctatggccaaacttccattattttaatcagtatgctgtatacagcgacgtattttggaatacctacaaaaaagtaaacaagaagtttgccaagaaacttgaggagataatcgaagacggggataagatttggattcatgactatcagttgttagttcttccggcaatgatcagagaaactcatcctaacagtagcattggattttttctccatatcccatttccttcctacgaatcatttagattattaccgtggagaacggatctcttgacaggtatgctgggagcagattttattggcttccacacctacgattatgtgcgtcactttctctcttctgtcaatagattggttggcataacggataatgatggtcacatgaatgtggggaataggttggctatggcagatgcaattcctatgggtattgattacaatcgatacgcacaggccgcagctgatcccgaaacactagcaagcgaggtaaagtatcgaatttctctgggtgatgtgaagttaatattatccatcgatagattggattattccaagggtataccccaaagattgcgagcttttgaacagtttatcgaagaaaatgaagaatttagggaagaggtttctttacttatactggtagttccatctcgagatcaggtgccgatgtatgccaatctcaagaaggaaatagaattgctagttggcagcatcaacggcaagtttggaactatcaactggaggccgatccattacttctacagaagctatcctttacatagcctaagtgccttttaccgaatgtcccacgtagcattggttactcctctaagggatgggatgaatctagtttgtaaagagtttgttgctagtaagttggacaaaaaaggtgtattaatattgagtgaaacggctggttctgccaaagagttgtcagacgcaattttaatcaatcccaatgatactaatcaaatggtcgaagccatgaaagaggctctgaagatgccagaggaggagcaaattgcacgaatggagaccatgcagaagtcattgaaaagatatgatatcaacgcttgggtaaaactcttcatgaagggattagaacaggtcaaagaggagcaagaaaatcttcggacaaaacccatttcttcagtggtcaaaaataaacttttgcaagaataccgtagctcaaaaaaacggatcattttccttgattatgacggcactttggtgggtttctatgctaatcctaatgactccgtaccagacgcagaattagaggagttgatgacgaagttaacggcagataccaacaatcaagtagttgtcatcagtggtagaggccgtgatttcttggagaaatggctctcaaaattcaatgttgatttcatcgccgagcatggggtttggcacaaagaaaatggaaaggagtgggagtgttttgtggaactagatacttcctggcaagaagaatttgatcgagtgctagagatgtatgtagatcgtacccctggctcttttatagagcgtaaggatttttccatggtatggcattacaggaaagtggagccaggtttgggtgaactaagatccagagaattggccaatcttttaaaatatttatccgccgacaaagatttacaagtccaagaaggtgatatggtcatcgaaattaaaaacgccagggtgaacaagggggttgctgccgcttcctggttgaagaaaaacgattacgatttctcttttgcctgtggtgatgactggaccgatgaggatacctttaaagccatgccagaggacgcatttaccgttaaagtcgggtcttcttcgtcggctgcaaaatatcgggttgagaactttaaggatatccgtaaactgttattgagcctagctaatcagtag

SEQ ID NO: 31 - protein, 726 aa, Crocosphaera watsonii
MSKTIIVSNRLPVKIERNQAGEFEYKTSEGGLATGLGSVYKEGDNIWVGWPGLAVNKTEDKEEICSRLKESNMSPVFLTKNEIEEYYEGFSNETLWPNFHYFNQYAVYSDVFWNTYKKVNKKFAKKLEEIIEDGDKIWIHDYQLLVLPAMIRETHPNSSIGFFLHIPFPSYESFRLLPWRTDLLTGMLGADFIGFHTYDYVRHFLSSVNRLVGITDNDGHMNVGNRLAMADAIPMGIDYNRYAQAAADPETLASEVKYRISLGDVKLILSIDRLDYSKGIPQRLRAFEQFIEENEEFREEVSLLILVVPSRDQVPMYANLKKEIELLVGSINGKFGTINWRPIHYFYRSYPLHSLSAFYRMSHVALVTPLRDGMNLVCKEFVASKLDKKGVLILSETAGSAKELSDAILINPNDTNQMVEAMKEALKMPEEEQIARMETMQKSLKRYDINAWVKLFMKGLEQVKEEQENLRTKPISSVVKNKLLQEYRSSKKRIIFLDYDGTLVGFYANPNDSVPDAELEELMTKLTADTNNQVVVISGRGRDFLEKWLSKFNVDFIAEHGVWHKENGKEWECFVELDTSWQEEFDRVLEMYVDRTPGSFIERKDFSMVWHYRKVEPGLGELRSRELANLLKYLSADKDLQVQEGDMVIEIKNARVNKGVAAASWLKKNDYDFSFACGDDWTDEDTFKAMPEDAFTVKVGSSSSAAKYRVENFKDIRKLLLSLANQ

SEQ ID NO: 32 - DNA, 2403 bp, Yarrowia lipolytica
atgctaccggaaatcatcaccccaaccgcggcgagagcgctcaatgtgcccatatctggacgggtcatcaactgtgtcacgaccctgccgtacgaaatctaccgcgagggagcgacgtacaaaattcgaccgcgacgtggcaactcggccctctactcggcgctggattatatgcagtctggcgacggagacaccacatggacatcgtcgctggtggcgtggacgggcgaaatcgcgctgccggccgccacgtcgctgccagacctggagctgtatcagaagctcacggagcaggacaaacacatgctcgaacgggagctgaccgaggcgcagggcggcacgccgactcacccgatctggacagactcgggcgacaccgtgtccacgggctacaacgaacagctgtcgcccacccgccgctacgcagaaaacattctgtggcccattctgcactacatccagggcgaacccaccgacgggcgcgacgagaaaaaatggtggagcgactacgaagacctcaaccgcaagtactgcgacaaggttctggacatctacaacgagggcgacgtcatctggatccacgactactacctgttcctgttgcccaaaatgatccgcgaaaagctgcccgacgcccggatcggcttcttcatgcatgcgccgttcccgtcgtcagagtactttcggtgtctggcaaagcgccaggagctgctgcagggcgtgctggcgtcgaatctcatttccacccagagcgaggcccacaaacggcactttatgagcgcatgttcccgcattgtgggcgcagaaacagccacgccaacgtccgtctatgcctacggccagtccgtgtccgtggtcgctctgccaatcggcatcgacacggcaaaggtggaggcggacgccttcactgatgaaataacggaaaaagtgcgggcgattcggcagctgtaccccgacaagaagatcattgtgggccgagaccggctggattcggtccggggcgtggtgcagaagttgtatgcgttcgacgtgtttctcaaacggtaccccgagtggcgggatcgcgtggtactggtgcaggtgacgtcacataccgccacaggcacgcgcaaagtcgaaaagaaggtggccgagctggtgtcatccataaacggcagatatggtgccatccacttttcgccagtgcatcattacaccaagcacattgctcgcgaggagtatctggctctgttgcgagtggcggacctttgtctcatcacttcggttcgtgatggcatgaacaccactgccttggagtttattgtgtgccaaaacggcaacaactctccgttgattctgtccgagttcacgggatcggccggaaacctgcctggcgcgattctggtgaacccctgggacgctgttggggttgcagagcagatcaaccggacattccgaatgggccaggacgagaagctggcgatcgagcaaccgctgtaccagcgggtgaccgccaacacggtgcagcactgggtgaaccggtttgtatcgcaggtgatcagcaacaccttccgaaccgaccagtctcatctgacgccgattctcgacaatcacaaacttgtggagcggttcaagatggccaaaaagcgagtgtttttgtttgattatgacggcactctcacgcccattgtgacggaccctgccgctgccactccttcagacggtctgaagcgggaccttcgagcgctggccaaggacccccggaacgccatatggataatttccggccgtgactccacgttcctggacaagtggctcggcgatattgctgaacttggcatgtctgccgagcatggctgtttcatgaagaatccaggcaccaccgactgggagaacttggcagccaactttgacatgagctggcagaaggacgtgaacgacattttccaatactacacggagcggacacaggggtcgcacattgaacgcaagcgtgtggctctgacatggcactaccgacgagcagaccctgaatttgggctgtttcaggcccgggagtgccgggcacatctggagcaggcggtggtgcccaagtgggacgtggaggtgatgagcggcaaggccaatcttgaagtacggcccaagtcggtcaacaagggtgagattgtcaaacggctcatttctgagtactcatcagagggccggcccccgcagtttgtcctgtgtatgggtgacgaccagacggacgaggacatgttcaaggctctcaaggatgtacctgatttggacagcgagagcattttccccgtaatgattgggcctccggagaagaagaccaccgccagctggcacctgctggagcccaagggcgtcctggagacgttgaatgagctggccaagttggagggcgagagtaagatgtag

SEQ ID NO: 33 - protein, 800 aa, Yarrowia lipolytica
MLPEIITPTAARALNVPISGRVINCVTTLPYEIYREGATYKIRPRRGNSALYSALDYMQSGDGDTTWTSSLVAWTGEIALPAATSLPDLELYQKLTEQDKHMLERELTEAQGGTPTHPIWTDSGDTVSTGYNEQLSPTRRYAENILWPILHYIQGEPTDGRDEKKWWSDYEDLNRKYCDKVLDIYNEGDVIWIHDYYLFLLPKMIREKLPDARIGFFMHAPFPSSEYFRCLAKRQELLQGVLASNLISTQSEAHKRHFMSACSRIVGAETATPTSVYAYGQSVSVVALPIGIDTAKVEADAFTDEITEKVRAIRQLYPDKKIIVGRDRLDSVRGVVQKLYAFDVFLKRYPEWRDRVVLVQVTSHTATGTRKVEKKVAELVSSINGRYGAIHFSPVHHYTKHIAREEYLALLRVADLCLITSVRDGMNTTALEFIVCQNGNNSPLILSEFTGSAGNLPGAILVNPWDAVGVAEQINRTFRMGQDEKLAIEQPLYQRVTANTVQHWVNRFVSQVISNTFRTDQSHLTPILDNHKLVERFKMAKKRVFLFDYDGTLTPIVTDPAAATPSDGLKRDLRALAKDPRNAIWIISGRDSTFLDKWLGDIAELGMSAEHGCFMKNPGTTDWENLAANFDMSWQKDVNDIFQYYTERTQGSHIERKRVALTWHYRRADPEFGLFQARECRAHLEQAVVPKWDVEVMSGKANLEVRPKSVNKGEIVKRLISEYSSEGRPPQFVLCMGDDQTDEDMFKALKDVPDLDSESIFPVMIGPPEKKTTASWHLLEPKGVLETLNELAKLEGESKM

SEQ ID NO: 34 - DNA, 2604 bp, Arabidopsis lyrata
  Arabidopsis lyrata subsp. lyrata
atggtgtcaagatcttgtgcaaattttatagatttagcatcttgggacttattggactttcctcaaactcaaagagctcttcctcgtgtcatgactgttcctggtatcatctctgagttggatggaggctacagtgatggatcctctgatgttaattcctcaagcagctcccgtgagcggaagattatagtggctaatatgttaccattacaagctaagagagatacagagagtggtcaatggtgttttagttgggatgaagattctcttctcttgcaactcagagatgggttttcttcggatacggagtttgtttatataggatcacttaatgctgatattggtacgagtgaacaagaagaagtttctcacaagcttttgttggatttcaattgtgttcctacgtttttacccaaggagatgcaagaaaagttctatcttggtttctgtaaacaccatttgtggccgctctttcactatatgcttcctatgttccctgaccacggtgatcgttttgaccggcgtctttggcaagcgtatgtctctgcaaacaagatattttcagatagggtgatggaagtcatcaaccctgaggaagattatgtttggattcatgattatcatctgatggttcttcccacattcttgaggaaacggtttaacaggatcaagcttggatttttccttcacagtccatttccatcgtcagaaatctaccgcactttgccagtgagggatgatcttctgagaggattgttgaactgtgatctcattggtttccacacatttgattatgcacgtcattttttgtcatgctgcagtagaatgcttggccttgattatgaatctaagcgtgggcacattgggcttgattactttggtcgaacggtgtttattaagatccttcctgttggcatccatatggggaggctggaatcggttttgaatcttccgtcgactgcagcgaaaatgaaagagatacaagaacagttcaaggggaaaaagttgattctcggtgtcgacgacatggacatctttaaaggcataagcctcaaacttatagccatggaacgtctctttgagacatattggcatatgcgaggaaaacttgtcctgattcagatagtgaacccagctcgggccacaggtaaggatgtggaagaagcaaagagggagacatattcaactgtaaaaaggattaacgagcgctatggttctgctggttatcagccagtgatcttgattgatcgtcttgttccacgttatgagaagactgcctattatgcaatggcagactgctgcctggtgaatgcagtaagagatggcatgaacttagttccatataaatatatcatttgcaggcaagggaccccaggaatggataaggccatgggcattagccatgactcaccccggacgagcatgcttgtcgtctctgagtttatcggctgctcgccttcattgagtggtgcgatcagggtgaacccatgggatgtagatgctgtttcagaagcggtaaacttagccctcaccatgggtgaaactgaaaagcgattaaggcacgagaaacactatcactatgtcagtactcatgatgtgggttactgggcaaagagctttatgcaggatctggagagggcatgccgggaacattataataaacgttgttggggtattggttttggcttgagtttcagagttttgtcactatctccgagttttaggaagctatctatcgatcacattgtctcgacgtacagaactacacagagaagggcaatatttttggattatgacggcactctcgttcctgagagctccattatcaaaacccctaatgctgaagtcctgtctgttctgaaatctctgtgtggagatcctaaaaacactgtgtttgtcgtcagtggaagaggatgggagtctctgagcgactggctatctccatgtgaaaatcttggaatcgcagctgaacacggatacttcataaggtggagtagcaagagagagtgggagacttgttactcgtcggctgaggcggaatggaagacgatggtagaaccggtaatgagatcatacatggacgcaacggatggttctactatagagttcaaagagagtgctttggtttggcatcatcaagaagcggatccggactttggagcctgtcaagcaaaggagcttctggatcatctagagagtgtacttgcaaatgagcctgttgtcgtcaagagaggccaacacattgtagaggtcaaaccacagggagtgagcaaaggtctagccgtggaaaaggtgatacaccgaatggtagaggatggaaacccaccggacatggtaatgtgtataggagatgacagatcagacgaggacatgtttgagagcatattgagcacagtgacaaacccggacctcccaatgccaccagagatctttgcttgcacggtgggaagaaaaccaagcaaagccaagtacttcttagatgatgtctcagatgtattgaagctcctaggaggattagctgctgcctcgagcagcaggaagccagaggatcaacaacaatcctcctcattgcacacgcaagtggcgtttgagagcatcatctga

SEQ ID NO: 35 - protein, 867 aa, Arabidopsis lyrata
  Arabidopsis lyrata subsp. lyrata
MVSRSCANFIDLASWDLLDFPQTQRALPRVMTVPGIISELDGGYSDGSSDVNSSSSSRERKIIVANMLPLQAKRDTESGQWCFSWDEDSLLLQLRDGFSSDTEFVYIGSLNADIGTSEQEEVSHKLLLDFNCVPTFLPKEMQEKFYLGFCKHHLWPLFHYMLPMFPDHGDRFDRRLWQAYVSANKIFSDRVMEVINPEEDYVWIHDYHLMVLPTFLRKRFNRIKLGFFLHSPFPSSEIYRTLPVRDDLLRGLLNCDLIGFHTFDYARHFLSCCSRMLGLDYESKRGHIGLDYFGRTVFIKILPVGIHMGRLESVLNLPSTAAKMKEIQEQFKGKKLILGVDDMDIFKGISLKLIAMERLFETYWHMRGKLVLIQIVNPARATGKDVEEAKRETYSTVKRINERYGSAGYQPVILIDRLVPRYEKTAYYAMADCCLVNAVRDGMNLVPYKYIICRQGTPGMDKAMGISHDSPRTSMLVVSEFIGCSPSLSGAIRVNPWDVDAVSEAVNLALTMGETEKRLRHEKHYHYVSTHDVGYWAKSFMQDLERACREHYNKRCWGIGFGLSFRVLSLSPSFRKLSIDHIVSTYRTTQRRAIFLDYDGTLVPESSIIKTPNAEVLSVLKSLCGDPKNTVFVVSGRGWESLSDWLSPCENLGIAAEHGYFIRWSSKREWETCYSSAEAEWKTMVEPVMRSYMDATDGSTIEFKESALVWHHQEADPDFGACQAKELLDHLESVLANEPVVVKRGQHIVEVKPQGVSKGLAVEKVIHRMVEDGNPPDMVMCIGDDRSDEDMFESILSTVTNPDLPMPPEIFACTVGRKPSKAKYFLDDVSDVLKLLGGLAAASSSRKPEDQQQSSSLHTQVAFESII

SEQ ID NO: 36 - DNA, 2571 bp, Arabidopsis lyrata
  Arabidopsis lyrata subsp. lyrata
atggtgtcaagatcttgtgctaattttctagacatatcatcttgggaccttttagattttcctcaaactccaagaactcttccacgcttcatgactgtccccggaatcatcaccgacgtagacggaggagatataacctccgaagtaacttcatcctccggtggctcacgtgagaggaagatcattgttgctaatatgttaccacttcaatccaaaagagatacagaaactggtaaatggtgttttcattgggacgaagactctctccagttacaacttagagatgggttttcttcagaaacagagtttctctacgttggatcacttaacgttgatatcgaaacgagtgagcaagaagaagtttcacaaaggcttttagaggaatttaactgcgttgcaacgtttttgtctcaagagttgcaagaaatgttctatcttggtttttgtaaacatcagttatggccactctttcattacatgcttccaatgtttcctgatcatggagatcgtttcgaccgacgtttatggcaagcttatgtgtctgctaacaagatattttcagacagagttatggaagttatcaaccctgaggatgattatgtttggattcaagattatcatctcatggttcttcctactttcttgaggaaacgttttaataggattaaactcgggtttttccttcatagtccgtttccttcttcagagatttaccgcacattgcctgttcgtgacgagattctgagaggtttgttgaattgtgatcttattggattccatacgtttgattacgcgcggcatttcttgtcatgttgtagtagaatgcttggtcttgattatgagtctaagcgcggtcatatagggcttgattactttggtaggactgtgtatatcaaaattcttcctgttggtgttcatatgggtagattggaatctgttttgaatcttgattctactgcggcgaaaactaaagagattcaagaacagtttaaagggaaaaaactggttcttggtatcgatgatatggatatatttaaaggtataagcttaaagcttatagcaatggaacatctcttcgagacttattggcatttgagagggaaagttgttcttgttcagatagtgaatcctgcaagatcctctggtaaagatgtggaagaagcgaaaagagagacgtatgtgactgcgaaaaggatcaatgagcgttacggtacttctgattataagccgatagtcttgatcgatcgtcttgttccacgttctgagaaaaccgcgtattatgctgcagcagattgttgcttggtgaatgcagtgagagatggtatgaacttagttccttataagtatatagtctgcagagaagggactcgaaacaaggcccttgatgattcatcaccccgcacaagcacgcttgttgtgtctgagtttattggatgctcgccttctttgagtggtgccattagggtgaatccatgggatgtggatgctgtggctgaggcggtaaactcggctctgaaaatgagtgagacagagaagcaactacggcatgagaaacattaccactatattagcactcatgatgtgggttactgggcaaagagctttatgcaggatcttgagagagcttgccgggatcattatagtaaacgttgttgggggattggatttggattggggttcagagttttgtcgctctctccaagtttcaggaagctatccgtggaaaacattgtcccggtttatagaaaaacacagagaagggcgatatttcttgattatgatggcactcttgttcctgaaagctccattgttcaagatccaagcgccgaggttgtctctgttctgaaagctctctgtgaagatcccaataacacagtgtttattgttagtggaagaggaaaagagtctctgagcaattggctatctccttgtgaaaatcttggaatagcggctgaacatggatacttcataaggtggaatagcaaagatgagtgggagacttgttactcgccttcggatacagagtggaggtcattggtggaaccggttatgagatcgtatatggaggcaacggatggaacgagtatagagtttaaagaaagtgctttggtgtggcaccatcaagacgcagatccggactttggatcatgtcaagctaaggagatgcttgatcatctagagagtgttctcgccaatgagcctgtcgttgtaaagagaggtcaacacatcgttgaagtcaaaccacagggtgtaagcaaaggtttagctgcggaaaaggtaatccgaggaatggtagaacgcggggagccaccggaaatggtgatgtgcataggagacgatagatcagacgaagacatgtttgagagcatattaagcacagtgacaaatccagagcttcttgttcagccagaggtttttgcatgcacggttggaagaaaaccaagcaaagctaaatacttcttagacgatgaagccgacgtgcttaagctcctaagaggccttggagactcatcatcaagcttaaaacccacatcttctcacacacaagtttcatttgaaagcatcgtttaa

SEQ ID NO: 37 - protein, 856 aa, Arabidopsis lyrata
  Arabidopsis lyrata subsp. lyrata
MVSRSCANFLDISSWDLLDFPQTPRTLPRFMTVPGIITDVDGGDITSEVTSSSGGSRERKIIVANMLPLQSKRDTETGKWCFHWDEDSLQLQLRDGFSSETEFLYVGSLNVDIETSEQEEVSQRLLEEFNCVATFLSQELQEMFYLGFCKHQLWPLFHYMLPMFPDHGDRFDRRLWQAYVSANKIFSDRVMEVINPEDDYVWIQDYHLMVLPTFLRKRFNRIKLGFFLHSPFPSSEIYRTLPVRDEILRGLLNCDLIGFHTFDYARHFLSCCSRMLGLDYESKRGHIGLDYFGRTVYIKILPVGVHMGRLESVLNLDSTAAKTKEIQEQFKGKKLVLGIDDMDIFKGISLKLIAMEHLFETYWHLRGKVVLVQIVNPARSSGKDVEEAKRETYVTAKRINERYGTSDYKPIVLIDRLVPRSEKTAYYAAADCCLVNAVRDGMNLVPYKYIVCREGTRNKALDDSSPRTSTLVVSEFIGCSPSLSGAIRVNPWDVDAVAEAVNSALKMSETEKQLRHEKHYHYISTHDVGYWAKSFMQDLERACRDHYSKRCWGIGFGLGFRVLSLSPSFRKLSVENIVPVYRKTQRRAIFLDYDGTLVPESSIVQDPSAEVVSVLKALCEDPNNTVFIVSGRGKESLSNWLSPCENLGIAAEHGYFIRWNSKDEWETCYSPSDTEWRSLVEPVMRSYMEATDGTSIEFKESALVWHHQDADPDFGSCQAKEMLDHLESVLANEPVVVKRGQHIVEVKPQGVSKGLAAEKVIRGMVERGEPPEMVMCIGDDRSDEDMFESILSTVTNPELLVQPEVFACTVGRKPSKAKYFLDDEADVLKLLRGLGDSSSSLKPTSSHTQVSFESIV

SEQ ID NO: 38 - protein, 12 aa, Artificial sequence
  TPS homolog protein motif
GXFXHXPFPSXE

SEQ ID NO: 39 - protein, 8 aa, Artificial sequence
  TPS homolog protein motif
XXXXXXXX

SEQ ID NO: 40 - protein, 15 aa, Artificial Sequence
  TPS homolog protein motif
XXXRHFXSXXXRXXG

SEQ ID NO: 41 - protein, 7 aa, Artificial Sequence
  TPS homolog protein motif
XXXXXXG

SEQ ID NO: 42 - protein, 20 aa, Artificial Sequence
  TPS homolog protein motif
XSEXXGXXXXLXXAXXXNPX

SEQ ID NO: 43 - protein, 6 aa, Artificial Sequence
  TPS homolog protein motif
XDYDGT

SEQ ID NO: 44 - protein, 6 aa, Artificial Sequence
  TPS homolog protein motif
AEHGXX

SEQ ID NO: 45 - protein, 9 aa, Artificial Sequence
  TPS homolog protein motif
XXLXXXXXX

SEQ ID NO: 46 - protein, 11 aa, Artificial Sequence
  TPS homolog protein motif
XEXXXXXXXKG

SEQ ID NO: 47 - protein, 10 aa, Artificial sequence
  TPS homolog protein motif
GDDXXDEXXF

SEQ ID NO: 48 - protein, 6 aa, Artificial sequence
  TPS homolog protein motif
XXXXXG

SEQ ID NO: 49 - protein, 11 aa, Artificial sequence
  TPS homolog protein motif
XXXXAXXXXXX

SEQ ID NO: 50 - DNA, 2571 bp, Artificial sequence
  codon optimized AtTPS8 for expression in Zea mays
atggtcagccgatcctgtgccaacttcctcgatctctcgagttgggatctgcttgatttccctcaaacaccgaggactctcccacgcgttatgaccgtacccggtatcattacggacgttgatggcgatactacaagcgaggtcacgtcaacctcaggaggttcacgtgagcggaagattatagtcgccaatatgcttccacttcaatccaaacgcgacgcggaaaccgggaagtggtgcttcaattgggatgaggattcactccagttgcaacttcgcgacggtttctcttcggagacggagttcctttacgtcggatctctgaacgttgatatcgaaacaaacgagcaagaggaagtcagccaaaagctgctcgaagagttcaactgcgtggctacattcctctcccaagaactccaagagatgttctaccttggtttctgcaaacaccaactgtggccactcttccactacatgttaccgatgtttcccgaccatggcgataggttcgaccgacgcctctggcaagcgtacgtgagtgcaaacaagatcttctccgatagggtgatggaggtgataaacccagaggacgattacgtctggattcaagactaccaccttatggtactccctacgttccttaggaagaggttcaaccgtatcaagcttggtttctttcttcactcaccctttccctctagcgaaatctataggacccttccggtcagagacgagatccttagagggctgctcaactgtgacctaatcggatttcataccttcgactacgctcgccatttcctatcgtgctgttcgagaatgctcggtctggattacgagtctaagcgtgggcacatcggacttgactactttggacggacggtctacatcaagattcttccagtgggtgttcacatgggacgcctcgaatctgttctttcgctcgactccaccgcagccaagaccaaggagattcaagaacagttcaaggggaagaaactggtccttgggattgatgacatggatatcttcaagggaatatcccttaagttgatcgcaatggagcacctgtttgaaacctattggcacctcaaaggcaaggtggtactcgtccaaatcgtgaaccctgctcgaagttccggaaaagacgttgaggaagcgaaacgcgagacctatgaaacagcacgacggatcaatgagcgctacggcacttcggactataaacccatcgtgctgattgatcgcttggtccctagatccgaaaagaccgcttactatgctgccgctgattgctgcctcgtcaatgcggtgcgtgacggtatgaacctagtcccttataagtacatcgtctgtcgtcaaggaacacgcagcaacaaggcagtagtcgattcgtcccctcggacgagcactctggtggtttctgaattcatcggctgctcaccctcgctgtccggagcgattcgcgtcaatccttgggatgtggatgctgttgcggaagccgttaacagcgctttgaagatgagtgagacggagaaacagctaagacatgagaaacactaccactacatttctacacacgacgtagggtattgggctaagtcgttcatgcaagacctggagagggcctgtagggatcactacagtaaacggtgctggggtataggattcggacttggcttccgcgtactttccctatccccttcctttcgcaaactcagcgtggaacatatcgttccggtctaccggaagacgcaacggagagcgatcttcttggactatgatggcaccctggttcccgaatcaagtatagttcaagacccgagtaatgaggtggtttcggttctgaaggctctctgcgaagaccccaataacaccgtcttcatagttagcggacgtgggagggagtccttaagtaactggctgagtccctgcgagaaccttgggattgctgcggagcacggttacttcattaggtggaagtcgaaagatgaatgggagacgtgctactcccctaccgacaccgagtggcgtagtatggttgaaccagttatgaggagctatatggaagcgactgacggcacctccattgagttcaaggagtctgcgttggtctggcaccaccaagatgccgaccctgactttggaagctgccaagcaaaggagatgttagaccatttggaaagcgtccttgcaaatgagccagtcgtggtcaagaggggtcagcatatcgtggaagtgaaaccccaaggcgtctcgaagggactagctgcggagaaagtgatacgagaaatggtcgaacgtggggaacctccggagatggtaatgtgtatcggagatgaccgttccgacgaggatatgttcgagagtatccttagcactgtgacgaatccggagctgctcgtacagcctgaggtgtttgcctgcactgtgggtcggaagccaagtaaggctaagtacttcctggacgatgaagctgatgtgttgaaacttctcagagggcttggcgactcaagctcgagcttgaagccttcttcctcacacacccaagtagcgtttgaatctatcgtctga

SEQ ID NO: 51 - DNA, 2604 bp, Artificial sequence
  codon optimized AtTPS9 for expression in Zea mays
atggtaagccgtagctgtgccaactttttggatctagccagctgggatcttctcgatttcccccaaactcagcgtgctctccctcgcgttatgacggttcccggcatcatttcagaactggacggcggttacagtgacgggtcttcagatgtcaattcatctaactcttcacgggagcggaagattatagttgctaacatgctgcctctccaagctaagcgcgataccgagacgggccagtggtgcttctcctgggatgaggatagccttctgctgcaactgcgggacggcttttcctcggataccgagttcgtctatatcggctccctaaacgctgatattgggatctccgagcaagaggaagtgtcgcacaagctcttgcttgacttcaactgcgtcccaactttcctacccaaggagatgcaagagaagttctacttgggcttctgcaaacaccatctctggccactcttccactacatgctcccaatgttcccggaccacggagacagattcgacagacgcctttggcaagcgtacgtgtcagcaaacaagatctttagcgaccgcgtaatggaggtgattaacccggaagaggactacgtgtggattcacgactaccacttgatggtgcttccgacatttctgaggaagcgctttaatcggatcaagttggggtttttccttcactcgcccttccccagctccgaaatctataggacactccccgtccgtgatgacctgcttcgtggcctgttgaattgcgatctcatcggctttcacacattcgactacgctaggcacttcctgtcctgttgctcgagaatgctgggcctagactacgagtctaaacgggggcatatcgggctcgactacttcgggagaacggtctttatcaagatacttccggtgggaattcacatgggacgcctcgagtcagttctaaacttgcccagcacagccgctaagatgaaagaaatccaagagcagttcaaaggcaaaaagcttatcctcggggtcgacgatatggatatcttcaaggggatttccctcaagctgatcgcgatggaaaggttattcgagacttactggcatatgaggggaaagctcgttctgatccaaatagttaacccggcaagggccaccggaaaggacgtcgaggaagcaaagaaagaaacctacagcaccgcaaagaggatcaacgaacgttacggctctgctggctaccaacccgttattctgatagatcgtttggtcccacgatacgagaagactgcgtactatgcgatggcggactgttgcttagtcaacgcggtgcgagacggcatgaatctggtgccctacaagtatatcatatgccgacaaggcacgccagggatggacaaggcgatgggtatatctcacgatagcgcacggacctctatgctggtggtcagtgagttcatcggttgctccccgtctttgagcggggctatacgggtgaacccttgggacgttgacgccgtagctgaagctgtcaacttggcgttaacaatgggcgagaccgagaaacgccttaggcacgagaagcattaccactacgtctccacgcatgacgttggttactgggctaagagctttatgcaagacctcgaacgggcatgccgcgagcactacaataagcggtgttggggcatagggttcggtttgagctttcgtgtgcttagtctaagcccaagcttccgaaagctctccatagatcacatcgtcagcacctacagaaacacgcagcgtagggccatattcttggactacgacggcactctcgttccggagagtagcatcataaagacgcctaatgccgaggtactctccgtgctgaagagtctctgcggagacccgaagaataccgtcttcgtcgtatcgggaagaggttgggaatctctctctgactggctatcaccgtgcgaaaatctggggatcgcagctgagcacggttacttcattaggtggtcgagtaagaaagaatgggagacctgctattcgtctgctgaagccgagtggaagacaatggttgagccagttatgagaagctatatggatgcgacggacggctcgactatcgagtacaaggagtcagcgttagtgtggcatcaccaagacgctgacccagacttcggagcgtgtcaagctaaggagttgctcgaccatctcgaatccgttctagcgaacgagcctgtggtcgttaagcgtggtcagcatatcgttgaagtcaagccacaaggggtctccaagggtctcgccgtggagaaggtgatccaccaaatggtggaggacggcaaccctccggatatggtgatgtgtatcggggatgaccgctctgacgaggatatgttcgaatcaatccttagtacggtaacaaacccggatcttcctatgccacctgaaatcttcgcctgcaccgtgggacgcaaaccgagtaaagcgaagtattttctggatgacgtctcagatgtgctcaagcttctcggcggtctagctgcggccacttcgtccagtaaacctgagtaccagcaacagtcgtctagcctccacactcaagtcgccttcgagtctataatctga

SEQ ID NO: 52 - DNA, 1010 bp, Zea mays
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 53 - DNA, 2562 bp, Arabidopsis thaliana
tataataggcaacgactacttgtagtggctaacaggctcccagtttctgccgtgagaagaggtgaagattcatggtctcttgagatcagtgctggtggtctagtcagtgctctcttaggtgtaaaggaatttgaggccagatggataggatgggctggagttaatgtgcctgatgaggttggacagaaggcacttagcaaagctttggctgagaagaggtgtattcccgtgttccttgatgaagagattgttcatcagtactataatggttactgcaacaatattctgtggcctctgtttcactaccttggacttccgcaagaagatcggcttgccacaaccagaagctttcagtcccaatttgctgcatacaagaaggcaaaccaaatgttcgctgatgttgtaaatgagcactatgaagagggagatgtcgtctggtgccatgactatcatcttatgttccttcctaaatgccttaaggagtacaacagtaagatgaaagttggatggtttctccatacaccattcccttcgtctgagatacacaggacacttccatcacgatcagagctccttcgctcagttcttgctgctgatttagttggcttccatacatatgactatgcaaggcactttgtgagtgcgtgcactcgtattcttggacttgaaggaacacctgagggagttgaggatcaaggcaggctcactcgtgtagctgcttttccaattggcatagattctgatcggtttatacgagcacttgaggtccccgaagtcatacaacacatgaaggaattgaaagaaagatttgctggcagaaaggtgatgttaggtgttgatcgtcttgacatgatcaaagggattccacaaaagattctggcattcgaaaaatttctcgaggaaaatgcaaactggcgtgataaagtggtcttattgcaaattgcggtgccaacaagaactgacgttcctgagtatcaaaaactcacaagccaagttcatgaaattgttggacgcattaatggtcgttttgggacactgactgcagttccaatacatcatctggatcggtctctggactttcatgctttatgtgcactttatgccgtcacagatgttgcgcttgtaacatctttgagagatgggatgaatcttgtcagttatgagtttgttgcttgccaagaggccaaaaagggcgtcctcattctcagtggatttgcaggtgctgcacagtctctgggtgctggagctattcttgtgaatccttggaacatcacagaagttgctgcctccattggacaagccctaaacatgacagctgaagaaagagagaaaagacatcgccataattttcatcatgtcaaaactcacactgctcaagaatgggctgaaacttttgtcagtgaactaaatgacactgtaattgaggcgcaactacgaattagtaaagtcccaccagagcttccacagcatgatgcaattcaacggtattcaaagtccaacaacaggcttctaatcctgggtttcaatgcaacattgactgaaccagtggataatcaagggagaagaggtgatcaaataaaggagatggatcttaatctacaccctgagcttaaagggcccttaaaggcattatgcagtgatccaagtacaaccatagttgttctgagcggaagcagcagaagtgttttggacaaaaactttggagagtatgacatgtggctggcagcagaaaatgggatgttcctaaggcttacgaatggagagtggatgactacaatgccagaacacttgaacatggaatgggttgatagcgtaaagcatgttttcaagtacttcactgagagaactcccaggtcacactttgaaactcgcgatacttcgcttatttggaactacaaatatgcagatatcgaattcgggagacttcaagcaagagatttgttacaacacttatggacaggtccaatctctaatgcatcagttgatgttgtccaaggaagccgctctgtggaagtccgtgcagttggtgtcacaaagggagctgcaattgatcgtattctaggagagatagtgcatagcaagtcgatgactacaccaatcgattacgtcttgtgcattggtcatttcttggggaaggacgaagatgtttacactttcttcgaaccagaacttccatccgacatgccagccattgcacgatccagaccatcatctgacagtggagccaagtcatcatcaggagaccgaagaccaccttcaaagtcgacacataacaacaacaaaagtggatcaaaatcctcatcatcctctaactctaacaacaacaacaagtcctcacagagatctcttcagtcagagagaaaaagtggatccaaccatagcttaggaaactcaagacgtccttcaccagagaagatctcatggaatgtgcttgacctcaaaggagagaactacttctcttgcgctgtgggtcgtactcgcaccaatgctagatatctccttggctcacctgacgacgtcgtttgcttccttgagaagctcgctgacaccacttcctcacct

SEQ ID NO: 54 - protein, 854 aa, Arabidopsis thaliana
YNRQRLLVVANRLPVSAVRRGEDSWSLEISAGGLVSALLGVKEFEARWIGWAGVNVPDEVGQKALSKALAEKRCIPVFLDEEIVHQYYNGYCNNILWPLFHYLGLPQEDRLATTRSFQSQFAAYKKANQMFADVVNEHYEEGDVVWCHDYHLMFLPKCLKEYNSKMKVGWFLHTPFPSSEIHRTLPSRSELLRSVLAADLVGFHTYDYARHFVSACTRILGLEGTPEGVEDQGRLTRVAAFPIGIDSDRFIRALEVPEVIQHMKELKERFAGRKVMLGVDRLDMIKGIPQKILAFEKFLEENANWRDKVVLLQIAVPTRTDVPEYQKLTSQVHEIVGRINGRFGTLTAVPIHHLDRSLDFHALCALYAVTDVALVTSLRDGMNLVSYEFVACQEAKKGVLILSGFAGAAQSLGAGAILVNPWNITEVAASIGQALNMTAEEREKRHRHNFHHVKTHTAQEWAETFVSELNDTVIEAQLRISKVPPELPQHDAIQRYSKSNNRLLILGFNATLTEPVDNQGRRGDQIKEMDLNLHPELKGPLKALCSDPSTTIVVLSGSSRSVLDKNFGEYDMWLAAENGMFLRLTNGEWMTTMPEHLNMEWVDSVKHVFKYFTERTPRSHFETRDTSLIWNYKYADIEFGRLQARDLLQHLWTGPISNASVDVVQGSRSVEVRAVGVTKGAAIDRILGEIVHSKSMTTPIDYVLCIGHFLGKDEDVYTFFEPELPSDMPAIARSRPSSDSGAKSSSGDRRPPSKSTHNNNKSGSKSSSSSNSNNNNKSSQRSLQSERKSGSNHSLGNSRRPSPEKISWNVLDLKGENYFSCAVGRTRTNARYLLGSPDDVVCFLEKLADTTSSP

SEQ ID NO: 55 - DNA, 2934 bp, Sorghum bicolor
atgagctctgacgccgcggggggacagcgcagcatcagcaactccacgaggggcgacgcggcggcggcgatgccaacctcatcgccctttgtcgtcggcgacagcagcggcggcgcgggctccccgatccgcgtcgaccgaatggtccgggagcacggccgccgctacgacatcttcgcgtcggacgcgatggataccgacggcgccgagccggcgtcggcttccgcggggcccttcgccgtggatggggtccagtcgcctggccgtgtgtcacccgccaacatggaggatgccggcggcgcggccgctgggcacgccgcgcgaccgccgctcgccggctcccgcagcggtttccgccgcctcggcctccgtggcatgaagcagcgcctcctcgtcgtggccaaccgcctccctgtttccgccaaccgccgcggcgaggaccactggtcgcttgagatcagcgccggcggcctcgtgagcgccctgcttggggtgaaggacgtcgacgcgaaatggattggctgggcgggcgtcaacgttccagacgaggttggccagcgagccctcaccaaagctcttgccgagaagagatgcataccagtgttcctggatgaggagattgtgcaccagtactacaatgggtattgcaacaacatcctgtggccgctgttccactacctaggactaccacaggaggacaggctggcaacaacgaggaactttgagtcacagttcgacgcgtacaagcgtgctaaccagatgtttgctgatgtcgtgtaccagcactaccaggagggggatgtaatctggtgccatgactaccacctcatgttcctgcccaagtgcctcaaggaccatgacatcaatatgaaagtcggttggttcctgcacacgccattcccatcatcagagatttaccgaacactgccatcccgcttggagctgcttcgctcggtgctgtgtgctgatttagtcggatttcatacttacgactatgcgaggcattttgtgagtgcttgcactagaatacttggacttgagggtacccctgagggtgtggaagatcaaggaagactaaccagggttgcagcgtttcctattgggatagactctgatcgtttcaagcgagcattggagcttccagcagtaaaaaggcacatcagtgaattgacacaacgttttgctggtcgaaaggtaatgcttggtgttgatcgacttgacatgattaagggaattccacaaaagattttggcctttgaaaagtttcttgaggaaaacccagactggaacgacaaagttgttctactgcagattgctgtgccaacaagaactgacgtccctgagtatcaaaagctaacaagccaagtgcatgaaattgttgggcgcataaacggtcgattcggaacgttgactgctgtccctattcatcatctggaccgatctcttgatttccatgctttgtgtgctctttatgcagtcactgatgttgctcttgtaacatcactgagagatgggatgaaccttgtgagctatgagtatgttgcatgccaagggtctaagaaaggagttttgatacttagtgagtttgctggggcagcacaatcacttggagctggcgccattctagtaaacccttggaatattacagaagttgcagactcaatacggcacgctttgacgatgccatccgatgagagagagaaacggcacaggcacaactatgctcatgtcacaactcacacggctcaagattgggctgaaacttttgtatttgagctaaatgacacggttgctgaagcactactgaggacaagacaagttcctcctggacttcctagtcaaacggcaatccagcaatatttgcgctctaaaaatcgtctgctcatattgggtttcaattcaacattgactgaaccagtcgaatcctctgggagaaggggtggtgaccaaatcaaggaaatggaactcaagttgcatcctgacttaaagggtcctctgagagccctctgtgaagatgagcgcactacagttattgttcttagtggcagtgacaggagtgttcttgatgaaaatttcggagaatttaaaatgtggttggcagcagagcatgggatgtttttacgcccgacttatggagaatggatgacaacaatgcctgagcatctgaacatggattgggttgacagcgtaaagcatgtttttgaatactttacagaaagaaccccaagatcccatttcgaacatcgtgaaacatcatttgtgtggaactacaagtatgctgatgttgaatttggaaggctacaagcaagagatatgctgcagcacttgtggacaggtccgatctcaaatgcagctgttgatgttgttcaagggagtcggtcagttgaagttcggtctgttggagttacaaagggtgctgcaattgatcgcattttaggggagatagttcacagcgaaaacatggttactccaattgactatgtcctgtgtatagggcatttccttgggaaggatgaggatatctatgtcttttttgatcccgagtacccttctgaatccaaaataaaaccagagggtggctcagcttcacttgaccggaggcccaacggaaggccaccatcgaacggcaggagcaactccaggaacccacagtccaggacacagaaggcgcagcaggctcaggctgcatccgagaggtcatcctcttcaagccacagcagcgcaagcagcaaccatgactggcgcgaagggtcctcggtccttgatctcaagggcgagaactacttctcctgcgccgttggaaggaaacggtccaacgcccgctacctgctgagttcgtcagaggaggttgtctccttcctcaaggagctggcaacagcaacagctggcttccaatccagctgtgctgattacatgttcctggataggcagtaa

SEQ ID NO: 56 - protein, 977 aa, Sorghum bicolor
MSSDAAGGQRSISNSTRGDAAAAMPTSSPFVVGDSSGGAGSPIRVDRMVREHGRRYDIFASDAMDTDGAEPASASAGPFAVDGVQSPGRVSPANMEDAGGAAAGHAARPPLAGSRSGFRRLGLRGMKQRLLVVANRLPVSANRRGEDHWSLEISAGGLVSALLGVKDVDAKWIGWAGVNVPDEVGQRALTKALAEKRCIPVFLDEEIVHQYYNGYCNNILWPLFHYLGLPQEDRLATTRNFESQFDAYKRANQMFADVVYQHYQEGDVIWCHDYHLMFLPKCLKDHDINMKVGWFLHTPFPSSEIYRTLPSRLELLRSVLCADLVGFHTYDYARHFVSACTRILGLEGTPEGVEDQGRLTRVAAFPIGIDSDRFKRALELPAVKRHISELTQRFAGRKVMLGVDRLDMIKGIPQKILAFEKFLEENPDWNDKVVLLQIAVPTRTDVPEYQKLTSQVHEIVGRINGRFGTLTAVPIHHLDRSLDFHALCALYAVTDVALVTSLRDGMNLVSYEYVACQGSKKGVLILSEFAGAAQSLGAGAILVNPWNITEVADSIRHALTMPSDEREKRHRHNYAHVTTHTAQDWAETFVFELNDTVAEALLRTRQVPPGLPSQTAIQQYLRSKNRLLILGFNSTLTEPVESSGRRGGDQIKEMELKLHPDLKGPLRALCEDERTTVIVLSGSDRSVLDENFGEFKMWLAAEHGMFLRPTYGEWMTTMPEHLNMDWVDSVKHVFEYFTERTPRSHFEHRETSFVWNYKYADVEFGRLQARDMLQHLWTGPISNAAVDVVQGSRSVEVRSVGVTKGAAIDRILGEIVHSENMVTPIDYVLCIGHFLGKDEDIYVFFDPEYPSESKIKPEGGSASLDRRPNGRPPSNGRSNSRNPQSRTQKAQQAQAASERSSSSSHSSASSNHDWREGSSVLDLKGENYFSCAVGRKRSNARYLLSSSEEVVSFLKELATATAGFQSSCADYMFLDRQ

SEQ ID NO: 57 - DNA, 2781 bp, Solanum lycopersicum
atgccagggaacaagtataccggcaaccaagcggttgctagcactcgattggagaggctattgagagaaagagagcttaggaaaagtagcaaagtttctcactttccaaatgaatctactgataacaataggggaaacgagctctctgaccatgattttcgccaaggagaagctgataatggaggagtttcatatgtcgaacagtacctcgaaggagctgcactagcatataatgaaggatgggagcggcctgatggaaagcccaccagacaacgactcttggttgtggcaaacaggttacctgtctctgcagtaaggagaggcgaggaatcctggtctctagagataagtggtggaggtcttgttagtgctcttcttggtgtgaaggagtttgaggctagatggattggttgggcaggtgtgaatgtgccagatgaggctgggcagagggcacttactaaggcactggcagaaaagaggtgtatccctgtattcctggatgaagaaattgttcatcagtattacaacggttactgcaacaatatattgtggcctcttttccattatcttggacttccgcaagaagaccgccttgcgactaccagaagtttccagtctcagtttgctgcttataagaaagcaaatcaaatgtttgctgatgttgtgaatgaacattacgaagaaggtgatgtggtatggtgtcatgactaccatctcatgttcctgccaaaatgtctcaaggattacaacagccaaatgaaagtcggttggtttctacacacaccctttccatcctcggaaatacacaggacactgccgtctagatcagagctgctccgagcagttcttgctgctgacttggttggttttcatacctatgactatgcaaggcattttgttagtgcatgtactcgtatcctgggacttgaaggaacacctgaaggagtagaagatcaaggtagactgacccgcgttgctgcgtttcctattggtatagattcagaacgatttattcgagcacttgaagttactcaagttcaggaacacataaaagaactaaaagagagatttgctgggagaaaggttatgctaggagttgatcgccttgatatgattaaaggaattccccaaaagatcctggcatttgagaagttccttgaagaaaatccgtactggcgtgataaagtggttttgcttcaaattgctgtgccaacaagaacagatgttcctgaataccaaaaacttaccagtcaagttcatgagattgttggacgcatcaatggtcggtttggaactttgactgcagtgcctattcatcatctggaccgttctcttgactttcatgcattatgtgcactgtatgctgtaactgatgtagcgttggttacctctttaagagatggcatgaacctcgtcagctatgaatttgtagcctgccaagagttgaaaaaaggggtccttattctcagcgaatttgctggtgctgcacaatcgttaggtgctggagcaattctggtgaatccatggaatataacagaggttgctgcttcgattgggcaagctttaaatatgtcagctgaagaaagagaaaaacgccacaggcataactttctgcatgtgactacgcatactgctcaagaatgggctgagacttttgtgagtgaactaaatgatactgttattgaagctcaacagaggataagaaaagttccgccccggcttaacatcagtgatgcaattgagcgctattcgttttccaataatcgactactaatattgggtttcaattctacactgacagaatcggtggatacccctggaagaagaggtggagatcaaatcaaagaaatggaactgaaattgcatcctgagttgaaagaatcattgctcgcgatttgtaacgacccaaagacaacagtcgttgtcctcagtggaagtgatagaaacgtcttagatgataacttcagcgagtacaacatgtggttagcagcagaaaatggaatgtttttacgatctacaaacggtgtatggatgacaactatgccagaacacctaaacatggactgggttgatagtgttaagcacgttttcgagtacttcactgaaaggacaccgagatctcactttgaacaacgtgaaacttcacttgtttggaattacaagtatgcagatgttgaatttggaagattgcaagctagagacatgcttcagcatctctggacaggtccaatatcaaatgcatctgttgatgttgtacaaggactccgctccgttgaggttcgagcagttggtgttacaaagggagcagcaatagatcgtatactgggggagatcgtacacagtaaagccatcgcaacaccaattgattacgttttatgcatagggcattttctggggaaggatgaggatgtatatacattttttgagccagagcttccttctgactgcatcggtatgccaagaagtaaggtcagtgatgcaccaaaggtgcccggggaaaggcgatcagttccaaaactcccttctagtcgaactagctcaaagtcatctcagaataggaacagaccagtttcaaactcggataagaagacttccaatgggcgacggccctcacctgaaaatgtgtcatggaatgtgctggatctgaagaaggagaattacttctcttgtgcagttggaaggactcgtacaaacgctcggtatctgctcagtacgccagacgacgttgttgcttttctaagggaactagctgaagcacctatttcaaatgggacatcatga

SEQ ID NO: 58 - protein, 926 aa, Solanum lycopersicum
MPGNKYTGNQAVASTRLERLLRERELRKSSKVSHFPNESTDNNRGNELSDHDFRQGEADNGGVSYVEQYLEGAALAYNEGWERPDGKPTRQRLLVVANRLPVSAVRRGEESWSLEISGGGLVSALLGVKEFEARWIGWAGVNVPDEAGQRALTKALAEKRCIPVFLDEEIVHQYYNGYCNNILWPLFHYLGLPQEDRLATTRSFQSQFAAYKKANQMFADVVNEHYEEGDVVWCHDYHLMFLPKCLKDYNSQMKVGWFLHTPFPSSEIHRTLPSRSELLRAVLAADLVGFHTYDYARHFVSACTRILGLEGTPEGVEDQGRLTRVAAFPIGIDSERFIRALEVTQVQEHIKELKERFAGRKVMLGVDRLDMIKGIPQKILAFEKFLEENPYWRDKVVLLQIAVPTRTDVPEYQKLTSQVHEIVGRINGRFGTLTAVPIHHLDRSLDFHALCALYAVTDVALVTSLRDGMNLVSYEFVACQELKKGVLILSEFAGAAQSLGAGAILVNPWNITEVAASIGQALNMSAEEREKRHRHNFLHVTTHTAQEWAETFVSELNDTVIEAQQRIRKVPPRLNISDAIERYSFSNNRLLILGFNSTLTESVDTPGRRGGDQIKEMELKLHPELKESLLAICNDPKTTVVVLSGSDRNVLDDNFSEYNMWLAAENGMFLRSTNGVWMTTMPEHLNMDWVDSVKHVFEYFTERTPRSHFEQRETSLVWNYKYADVEFGRLQARDMLQHLWTGPISNASVDVVQGLRSVEVRAVGVTKGAAIDRILGEIVHSKAIATPIDYVLCIGHFLGKDEDVYTFFEPELPSDCIGMPRSKVSDAPKVPGERRSVPKLPSSRTSSKSSQNRNRPVSNSDKKTSNGRRPSPENVSWNVLDLKKENYFSCAVGRTRTNARYLLSTPDDVVAFLRELAEAPISNGTS

SEQ ID NO: 59 - DNA, 2580 bp, Triticum aestivum
atgaagcagcgcctcctcgtcgtggccaaccgcctccccgtctccgccaatcgccgcggcgaggatcagtggtccctggagatcagcgccggtggcctcgtcagcgcgctcctcggtgtgaaagatgtcgacgcgaagtggatcggctgggccggtgtgaatgtccccgacgaggtcggccagcaggctctcaccaatgcactcgccgagaagagatgcataccagtcttcctggacgaggagatcgtgcaccagtactacaacggctactgcaacaacatactgtggccgctcttccactacctcgggctgccgcaggaggacaggctggcaaccacccggaacttcgagtcgcagttcgacgcgtacaagcgggccaaccagatgtttgctgatgtcgtctaccagcactaccaggaaggggatgtgatctggtgccatgactaccacctcatgttcctgcccaggtgcctcaaggagcatgacatcaacatgaaggtcgggtggttcctgcacacgcccttcccttcctcggagatttaccgcactctgccatcacgctcggagctgcttcgctccgtgctctgcgctgatttagtcggatttcatacatacgactatgcaaggcatttcgtgagcgcatgtaccagaatactcggactcgagggtacccctgaaggtgtggaggaccagggaaagttaacgcgggttgcagcgtttcctattgggatagactctgatcgtttcaaaagggcgttggacattgacgcagcaaaaagacatgtcaatgaactgaaacagcgatttgcgggacggaaggtaatgcttggtgttgatcgacttgacatgatcaaaggaattccccaaaagattttggcctttgaaaagtttcttgaggaaaaccctgaatggattgataaagtggttctacttcaaattgctgtgccaactagaactgacgtccctgagtatcagaagcttacaagccaagtgcatgaaattgttgggcgcataaatggacgatttggaacattgtctgctgttcctattcatcatctggatcgatctcttgatttccatgccttgtgtgctctttatgcagtcactgatgtggctcttgtaacatcactgagggatggcatgaatcttgtaagctacgaatatgttgcatgccagggatcaaaaaaaggagttctgatattgagtgagtttgccggtgcagcacaatcgcttggtgctggtgccattcttgtaaatccctggaatattacagaagttgcagactcaataaaacatgctttgacaatgacatctgatgagagagagaagcggcacaggcataactacgcgcatgtaacaactcataccgcccaagattgggctgaaacttttgtatgtgagctaaacgatacagttgctgaagctctgatgagaacaagacaagttccccctgaccttcctagtcgaacggccatccagcaatatctgcagtcaaaaaaccgtttgctcatattgggtttcaattcaacattgaccgagccagttgaatcctctgggagacggggcggtgatcaagtcaaggagatggaactcaagttgcatcctgacttaaagggtcctttgagagccctctgcgaggacgagagcactacggttatcgttctcagcggaagcgacaggagtgttcttgatgaaaatttcggagaatttaacttgtggctggcagcagagcatgggatgttcttacgcccaactgatggagaatggatgacaacaatgcctgagcatctgaacatggattgggtcgacagtgcaaagcatgtttttgagtacttcacagaaagaaccccaagatctcattttgaacatcgtgaaacatcatttgtgtggaattacaagtatgccgatgttgagtttgggaggctccaagcaagagatatgctgcagcacttgtggaccggtccaatctcaaatgcagctgtggatgttgttcaagggagccgttcagttgaagttcgctctgttggagttacaaagggtgctgcaattgatcgtattctaggagagatagttcacagcaaaagcatggttactccgattgactatgtgctatgcataggccacttcctaggaaaggacgaagacatctatgtgttttttgaccctgaatacccttctgagccaaaagtgaaaccggacggtgcgtcggtatccgtcgacaggaggcagaacgggcggccatcaaacggccggagcaactcgaggaactcgcaggcgaggacacaaaagcctcaggtcgcgccgccgcctccggagaggtcatcgtcgtcatccgaccacagcaccgcaaacaacaacagccaccacgactggcgcgaagggtcgtcggtcctcgacctcaacggcgacaactacttctcctgcgcggtcgggaggaagcgctccaacgcccgttacctgctcaactcgtcggaggacgtcgtctcattccttaaggagatggcggagtcgacgacgccccgcgccggtggcctcccgcccggcgctgccgcggactacatgttcttggataggcagtag

SEQ ID NO: 60 - protein, 859 aa, Triticum aestivum
MKQRLLVVANRLPVSANRRGEDQWSLEISAGGLVSALLGVKDVDAKWIGWAGVNVPDEVGQQALTNALAEKRCIPVFLDEEIVHQYYNGYCNNILWPLFHYLGLPQEDRLATTRNFESQFDAYKRANQMFADVVYQHYQEGDVIWCHDYHLMFLPRCLKEHDINMKVGWFLHTPFPSSEIYRTLPSRSELLRSVLCADLVGFHTYDYARHFVSACTRILGLEGTPEGVEDQGKLTRVAAFPIGIDSDRFKRALDIDAAKRHVNELKQRFAGRKVMLGVDRLDMIKGIPQKILAFEKFLEENPEWIDKVVLLQIAVPTRTDVPEYQKLTSQVHEIVGRINGRFGTLSAVPIHHLDRSLDFHALCALYAVTDVALVTSLRDGMNLVSYEYVACQGSKKGVLILSEFAGAAQSLGAGAILVNPWNITEVADSIKHALTMTSDEREKRHRHNYAHVTTHTAQDWAETFVCELNDTVAEALMRTRQVPPDLPSRTAIQQYLQSKNRLLILGFNSTLTEPVESSGRRGGDQVKEMELKLHPDLKGPLRALCEDESTTVIVLSGSDRSVLDENFGEFNLWLAAEHGMFLRPTDGEWMTTMPEHLNMDWVDSAKHVFEYFTERTPRSHFEHRETSFVWNYKYADVEFGRLQARDMLQHLWTGPISNAAVDVVQGSRSVEVRSVGVTKGAAIDRILGEIVHSKSMVTPIDYVLCIGHFLGKDEDIYVFFDPEYPSEPKVKPDGASVSVDRRQNGRPSNGRSNSRNSQARTQKPQVAPPPPERSSSSSDHSTANNNSHHDWREGSSVLDLNGDNYFSCAVGRKRSNARYLLNSSEDVVSFLKEMAESTTPRAGGLPPGAAADYMFLDRQ

SEQ ID NO: 61 - DNA, 2613 bp, Zostera marina
atgatgtcaagatcgtacacaaatcttctggatctagcgtcggggaacttcccggtgattagtggcggtggacgggatggtcgtagcggtgggatgcgacgaatgccgcgagtgatgactgttccgtcaaacatagcggagcttgaggatgaacaagcgagtagtgttgcttcggatgtgcagtcttctattattcaagatcggttaataatagtcggtaaccaacttcctgttgtcgccaaacgccgatcagataacgccggttgggatttctcttgggatgatgagtctctccttcttcaactcaaagacggcttaccggatgatatggaagtcttatacgtcggttgcctccgtgtcatcgtcgatcctgaagaacaagacgacgtctcccaaacactacttgagaagttcaaatgcgtgccggcatttctaactgaggaaatccttgaaaagtactatcacggcttctgcaagaagctactgtggccgttgtttcattacatgttgccattgacgaaagatcatggtggaaggtttgataggtctctttgggaggcttatgttgcggtgaacaagatattttcacagaaggttgttgaaattattagtccagaagatgactatgtttggattcatgattaccatctcatggttcttccaactttgcttagacgaaggtttattcggcttcgaatgggtttttttcttcacagcccgttcccatcatcagagatttatagaacacttcccgtacgtgaagagattctaaattcgctactatgttccgatttgattggattccacacatttgattatgcacggcatttcttgtcatgttgtagtagaatgatggggttggaataccaatcaaaacgagggtatatcagtttagattactttggccgaacggttggaatcaagatcatgcccgccagtattcatttgggccagttggagtctatgttgaagactgtgtataaggagtcgaagattgaggaacttgagaggcagtttcaggggaagactgtcattttaggagttgatgatatggatatctttaaggggattaatttgaaattattggccttcgaacagatgctgaagcttcgtcctaattggcagggaagggctgtgctggttcagatcgccaatcctgcaagagggagaggaaagggacttgaaagtgtggaggttgagattcgagatatttgtgaaaggatcaatcaacagtttggacgtgttggttacaaacctgtagtgtacatcaatcgatctgtttcattgaaagaaaggattgcctattacacaatcgcagaatgtgttgttgtttccgcagtgagggatgggatgaatctaataccatatgagtacactgtctgtaagcaaggaatcgccgagcctgaatcagattcattatttgctgatccaaaaaaaagtatgttggtcgtgtcagaattcattggttgttctccttctttgagtggtgcaatcaagattaatccttggaacagcgaagcgactgcagaggctatgagtgatgcaatctcgatgcccgatggagagaagcaattgcgtcatgggaagcattatagatatgttcggactcatggtgtttcatattggtcaaaaagtttcatgcaggatatggagaggacatgcaaggatcattttaagaggagatgttggggtattggattcgggtttggttttagagttgttgccctcgatcctaatttcaaaaaactcaatgtggactccattgtgttttcatatgaaagggccaaaagtagggctatattattggattatgatggaacgatgattaatccattatctattaacaaaacaccgagcactgaagtgatctctattttgaacgctcttagtaaagacaaaaagaatgttgtttttatggtgagtggcaggggaagggagagtttagggagttggttttcttcatgcgagaagcttggaattgcagcagagcatggttttttcatgaggtgggggcgagatgatgaatggacgacttgggacaaaaataaagattttgggtggatgttgatggcggatcctgtaatgaaattatacacagaggctacagatggatcatatattgaagccaaagaaagtgccttggtttggcaccaccgagatgccgatcaaacttttggaacctctcaagcaaaagagatgttagaccatctcgaaaacgttttggctaacgaacctgttatcgcaaagcgtggccaattcattgttgaagttaagccacagggagttagcaaaggtttagtagcagataacattttatcaacaatggctaagagaaactgcccagcagattttgtattgtgtattggtgatgatagatcagatgaagacatgtttgaaaactttggtagcaagaatttggtatcctttaatgcacatatatattcctgcacggttgggcaaaaacctagcaaagccacttattatttagatgacactaatgatgttttggaaatgcttcgtgcccttgctgatgcctccgaagaagatgatgacgatgaagaggaaatcgaagatgattatgttgatgatgaatcggaagaaggttcaagttaa

SEQ ID NO: 62 - protein, 870 aa, Zostera marina
MMSRSYTNLLDLASGNFPVISGGGRDGRSGGMRRMPRVMTVPSNIAELEDEQASSVASDVQSSIIQDRLIIVGNQLPVVAKRRSDNAGWDFSWDDESLLLQLKDGLPDDMEVLYVGCLRVIVDPEEQDDVSQTLLEKFKCVPAFLTEEILEKYYHGFCKKLLWPLFHYMLPLTKDHGGRFDRSLWEAYVAVNKIFSQKVVEIISPEDDYVWIHDYHLMVLPTLLRRRFIRLRMGFFLHSPFPSSEIYRTLPVREEILNSLLCSDLIGFHTFDYARHFLSCCSRMMGLEYQSKRGYISLDYFGRTVGIKIMPASIHLGQLESMLKTVYKESKIEELERQFQGKTVILGVDDMDIFKGINLKLLAFEQMLKLRPNWQGRAVLVQIANPARGRGKGLESVEVEIRDICERINQQFGRVGYKPVVYINRSVSLKERIAYYTIAECVVVSAVRDGMNLIPYEYTVCKQGIAEPESDSLFADPKKSMLVVSEFIGCSPSLSGAIKINPWNSEATAEAMSDAISMPDGEKQLRHGKHYRYVRTHGVSYWSKSFMQDMERTCKDHFKRRCWGIGFGFGFRVVALDPNFKKLNVDSIVFSYERAKSRAILLDYDGTMINPLSINKTPSTEVISILNALSKDKKNVVFMVSGRGRESLGSWFSSCEKLGIAAEHGFFMRWGRDDEWTTWDKNKDFGWMLMADPVMKLYTEATDGSYIEAKESALVWHHRDADQTFGTSQAKEMLDHLENVLANEPVIAKRGQFIVEVKPQGVSKGLVADNILSTMAKRNCPADFVLCIGDDRSDEDMFENFGSKNLVSFNAHIYSCTVGQKPSKATYYLDDTNDVLEMLRALADASEEDDDDEEEIEDDYVDDESEEGSS

SEQ ID NO: 63 - DNA, 2598 bp, Zea mays
atggtttcaaaatcatactcaaatctgctagacctgacctctggagatggatttgactttcgacaaccttttaagtctcttcctcgtgtcgtaacttctcctggtattatatctgacactgattgggatacaataagtgatggtgattcagttggttcagcatcttctactgagaggaaaataatcgttgccaacttccttccgttgaattgtacaagagatgaaactggggtgctttccttctcattggatcatgatgcgcttctcatgcaacttaaagatagtttttcaaatgagactgatgttgtgtatgtgggcagtttgaaggttcaggtagatcctggtgagcaggaccaagttgcacagaagcttcttagagaatatcgatgcatacctacttttctcccatctgacctacagcagcagttctatcatggcttctgtaaacaacaattatggccactttttcattatatgcttccaatttgccttgacaagggtgagctatttgatcgcagcctgtttcaagcttatgtccgagccaacaaactttttgcagataaagttatggaagcaatcaatgcagatgatgacttcgtttgggttcatgattatcatctcatgttgctccctacattcttgaggaagaggttacaccgaataaagattggtttcttccttcacagtccttttccctcctcagaaatctataggactctgcctgtaagggatgaaatcctgaagtcactgcttaatgctgatctcattggtttccaaacatttgactatgcccgccacttcctatcttgctgtagcagattgctaggcctgcattatgagtcaaaacgtggttacattggaatagagtattttggccgaacagtgagcctgaagatcctttccgtgggtgtccatattggtcggcttgaatctgtcttaaaattgcctgctacagttagtaaggttcaagaaattgaacaaaggtataagggcaagatactgatgttaggtgtagatgacatggacatcttcaaagggataagtctgaaatttcttggactggagcttcttctggacagaaacccaaagcttagagagaaggttgtccttgtacaaatcatcaatccagcaagaagcacagggaaggacgtgcaagaagctattacagaagctgtctctgtggctgaaaggattaatacaaattatggttcttcaagttacaagcctgttgtcctaattgatcaccacataccattttatgaaaagattgcattctatgctgcgtctgattgctgtattgtaaatgctgtgagggatggcatgaacttagtaccatatgagtatactgtttgccgacagggaaatgaggagattgataaactcagaggtcttggcaaagacacccatcacacaagcacacttattgtttcggagtttgtgggttgctccccatctcttagtggtgctttcagggtaaatccttggagtgtcgatgatgtggcggatgccttgtgccgtgcaactgatttgactgaatccgagaaacggctgcggcatgaaaagcattatcgctatgtcagtactcatgatgttgcttactgggcacgcagctttgctcaagatctggaaagagcatgcaaagatcattatagcagaaggtgttgggcgattggattcggtctgaattttagagttattgctctttctcctggcttcagaaagctgtcgtcagagcactttgtttcttcttataacaaggcttctagaagagcaatatttcttgattacgatggcacacttgtgccccagtcatcaatcaacaaagctccaagtgaagaagtcatttccgttcttaacaccttatgtaatgatccaaagaacattgtgtttatagtaagtggacgaggacgtgattcccttgatgagtggttttctccgtgtgagaagcttggtctagcggcagaacatggctattttatcagatggagcaaggaagccgcatgggagtcaagctattcaaggccgcagcaagaatggaagcacattgccgaacctgtgatgcaggtatacacagagacaacagatggatcttcaatcgagtcaaaggaaagcgccctagtatggcactatttggacgcggaccatgatttcggttccttccaagcaaaggagctacaaggtcatcttgagagggtgctatcgaatgagcctgttgttgtgaagtgtggtcattatattgtagaggtgaaaccacagggagttagcaaggggcttgctgtcaacaagctgattcacacactggtcaagaacgggaaggcaccggatttcctgatgtgcgtcggcaacgacagatctgatgaggacatgtttgaaagcatcaacggtatgacctccaacgctgtcttatcacccacaatgccggagctgtttgcctgttcagtcggtcagaagcccagcaaagcaaaatattatgtggacgacaccagcgaagtaatcagattgctcaagaatgtaacccgcatcccctcgcagcggcaggatgtcagtgccagccatgggcgtgtgaccttcagaggcgtgctcgattacgtggactag

SEQ ID NO: 64 - protein, 865 aa, Zea mays
MVSKSYSNLLDLTSGDGFDFRQPFKSLPRVVTSPGIISDTDWDTISDGDSVGSASSTERKIIVANFLPLNCTRDETGVLSFSLDHDALLMQLKDSFSNETDVVYVGSLKVQVDPGEQDQVAQKLLREYRCIPTFLPSDLQQQFYHGFCKQQLWPLFHYMLPICLDKGELFDRSLFQAYVRANKLFADKVMEAINADDDFVWVHDYHLMLLPTFLRKRLHRIKIGFFLHSPFPSSEIYRTLPVRDEILKSLLNADLIGFQTFDYARHFLSCCSRLLGLHYESKRGYIGIEYFGRTVSLKILSVGVHIGRLESVLKLPATVSKVQEIEQRYKGKILMLGVDDMDIFKGISLKFLGLELLLDRNPKLREKVVLVQIINPARSTGKDVQEAITEAVSVAERINTNYGSSSYKPVVLIDHHIPFYEKIAFYAASDCCIVNAVRDGMNLVPYEYTVCRQGNEEIDKLRGLGKDTHHTSTLIVSEFVGCSPSLSGAFRVNPWSVDDVADALCRATDLTESEKRLRHEKHYRYVSTHDVAYWARSFAQDLERACKDHYSRRCWAIGFGLNFRVIALSPGFRKLSSEHFVSSYNKASRRAIFLDYDGTLVPQSSINKAPSEEVISVLNTLCNDPKNIVFIVSGRGRDSLDEWFSPCEKLGLAAEHGYFIRWSKEAAWESSYSRPQQEWKHIAEPVMQVYTETTDGSSIESKESALVWHYLDADHDFGSFQAKELQGHLERVLSNEPVVVKCGHYIVEVKPQGVSKGLAVNKLIHTLVKNGKAPDFLMCVGNDRSDEDMFESINGMTSNAVLSPTMPELFACSVGQKPSKAKYYVDDTSEVIRLLKNVTRIPSQRQDVSASHGRVTFRGVLDYVD

SEQ ID NO: 65 - DNA, 2742 bp, Zea mays
atgatgtcgcggtcgtacaccaacctgctcgacctcgcggagggcaacttcgcggcgctgggcccggccgccggcgccggcggcagcgggcggcagaggcatgggtcgttcgggctgcggcggatgtcgcgggtgatgacggtgccggggacgctgacggagctcgacggcgaggacgagtcggagccggccgcgaccagcagcgtcgcctccgacgtgccctcgtccgtggcggcggaccgcctcatagtggtctcgaatcagttgcccatcgtcgcgcgccgcaggcccgacggccgagggtggtccttctcgtgggacgatgactcgctcctgctccagctccgcgacggcattcccgacgagatggaggtgctcttcgtcggatcactccgcgccgacgtccccgcagccgagcaggacgcggtatcgcaggcgctgctcgaccgattccgctgcgcgccggtgttcctccctgaccacctcaacgaccggttctaccacggcttctgcaagcgccagctctggcctctgttccactacatgctccccttctcatcgcccgcttccgcgtctgccgccgctacctcttcctccgtcgccacttcgtcacccggcaacggttgcttcgaccgcagcgcttgggaggcatacgtgctcgccaacaagttcttctttgagaaggtcgtcgaggtaatcaacccggaggatgactacgtctgggttcacgactaccatctcatggcgctgcctaccttcctccgccgctgcttcaaccgcctccgcatcggattcttcctccacagccccttcccctcgtccaagatctaccgcaccctccctgttcgggaggagatactcaaggcgctgctcaactgtgacctaattggcttccacacttttgattacgccaggcacttcctctcgtgctgcagtaggatgctgggaattgaataccagtcaaagcgtgggtacattgaattggattactttggccgcactgtcgggatcaaaatcatgccagtgggagttcatatgggtcaattggagttgggtctgcgcttgcctgatagggaatggaggctttctgagcttcaacgccagtttcaggggaaaactgtcttgcttggtgtggatgatatggatatctttaaggggatcaatttgaagcttctcgcctttgagaacatgttgaggacacatcccaagtggcaggggagagcagtgttagtgcagattgctaacccagcccgtggaaggggtaaagatctggaagccatccaggctgagattgaggagagctgccagcggatcaatggagactttggccagtcagggtatagccctgttgttttcatcgatcgtgatgtgtcaagtgttgagaagattgcctattatacgatagcggaatgtgtggtggtgactgctgtgagggatgggatgaacttgacaccgtatgaatacgttgtctgtaggcagggtgcaccaggatctcagtcggtagcagaggtgagtgggccaaagaagagcatgctggttgtgtcagagtttattggctgctcaccgtcactgagcggtgctattagggttaacccatggaatatagaggcaaccgcggaggcgatgaatgaggccatttcaatgccagaacaggaaaaacagttgaggcatgagaaacattaccgttatgtcaggagccatgacgtcgcttattggtcaaagagcttcatcatagacttggaaagggtttgtaaggatcacttcaagaggacttgttggggcatagggttgggttttggtttcagggtggtggccttggaccctcatttcacaaagcttaacatggattcaatcattaatgcttatgagctttcagagagcagggctatattgctcgattatgatggaactctggttccccaaacttccctcaacaaggaacctagtccacaggttttgagcatcatcaataccctttgctcagatagtagaaacatcgtttttcttgtcagtggtcgagacaaagataccttgggaaagtggttctcctcatgtccaagattggggattgcagctgaacatggttacttcttgaggtggtctagagaagaagagtggcaaacatgcactcaggcattggacttcggatggatgcaaatggcgaagccagtgatgaatttatatacagaagcaactgatggatcctacattgaggccaaggaaagtgccttggtgtggcaccatcaggatgctgacctaggctttggatcctcacaggcaaaggagatgcttgatcacctggaaagtgtactagcaaatgaaccagtctctgtcaagagtggccagttcattgttgaagtcaaaccacagggaataagcaaaggaatagttgctgagaggatacttgcatcagtgaaggagagaggaaagcaggctgatttcttattgtgcatcggcgatgataggtctgatgaggacatgtttgaaaatattgctgatatcactgggaggaatttggttgctccaagaacagcactgtttgcgtgcactgtgggacaaaaaccaagcaaagccaaattctatctggatgatacattcgaagtggtcactatgctgagcgcactggcagatgccacaggtcctgagactgattcggctgatgaatctgtcgcatatatctcatcacttgatattggtgacgaacaatcagaatccagtgataaaccagttgaagggtcttag

SEQ ID NO: 66 - protein, 913 aa, Zea mays
MMSRSYTNLLDLAEGNFAALGPAAGAGGSGRQRHGSFGLRRMSRVMTVPGTLTELDGEDESEPAATSSVASDVPSSVAADRLIVVSNQLPIVARRRPDGRGWSFSWDDDSLLLQLRDGIPDEMEVLFVGSLRADVPAAEQDAVSQALLDRFRCAPVFLPDHLNDRFYHGFCKRQLWPLFHYMLPFSSPASASAAATSSSVATSSPGNGCFDRSAWEAYVLANKFFFEKVVEVINPEDDYVWVHDYHLMALPTFLRRCFNRLRIGFFLHSPFPSSKIYRTLPVREEILKALLNCDLIGFHTFDYARHFLSCCSRMLGIEYQSKRGYIELDYFGRTVGIKIMPVGVHMGQLELGLRLPDREWRLSELQRQFQGKTVLLGVDDMDIFKGINLKLLAFENMLRTHPKWQGRAVLVQIANPARGRGKDLEAIQAEIEESCQRINGDFGQSGYSPVVFIDRDVSSVEKIAYYTIAECVVVTAVRDGMNLTPYEYVVCRQGAPGSQSVAEVSGPKKSMLVVSEFIGCSPSLSGAIRVNPWNIEATAEAMNEAISMPEQEKQLRHEKHYRYVRSHDVAYWSKSFIIDLERVCKDHFKRTCWGIGLGFGFRVVALDPHFTKLNMDSIINAYELSESRAILLDYDGTLVPQTSLNKEPSPQVLSIINTLCSDSRNIVFLVSGRDKDTLGKWFSSCPRLGIAAEHGYFLRWSREEEWQTCTQALDFGWMQMAKPVMNLYTEATDGSYIEAKESALVWHHQDADLGFGSSQAKEMLDHLESVLANEPVSVKSGQFIVEVKPQGISKGIVAERILASVKERGKQADFLLCIGDDRSDEDMFENIADITGRNLVAPRTALFACTVGQKPSKAKFYLDDTFEVVTMLSALADATGPETDSADESVAYISSLDIGDEQSESSDKPVEGS

SEQ ID NO: 67 - DNA, 2595 bp, Zea mays
atggttctgaagtcgcacacaaatctgctagatatgtgttgtgaagatgtgtttgacttccaacaacctttaagatctcctcgtcatgtggtgaactctcctggcatcatatctgaccctgattgggaaagtagtaatgatggcaactcagttggttctatgcctttttgttttaagagaaagattattgttgcaaatttccttcctgtgatctgtgcaaaaaatgaagctaccggagaatggtcctttgccatggatgataatcaactgcttgttcaactcaaagatggttttccaattggtaatgaagttatttatgtgggtagtttgaatgttcaagttgatcctattgagcaagatcgagtttctcagaagctcttcaaggaacacagatgcgtacctacctttctcccagctgaactccagcatcaattctatcacatattctgcaaacagcacttatggccacttttccattatatgcttcctgtttgtcatgacaaagatgagctctttgaccgttccctttttcaagcctacgtgcgggccaacaaaatttttgctgacaaaattgtggaggcagtcaattcggatgatgattgtgtgtgggttcatgattatcacctcatgcttatcccaacccttttaagaaagaaactgcaccggatcaaagttggtttcttcctccacagcccatttccctcgtctgagatctataggacactgccagtgcgggatgaaattctaaaatcacttcttaatgctgacctcattggctttcaaacttttgactatgcccgccacttcctttcatgttgcagcaggcttttaggccttaattatgagtccaaacgtggccacataggtatagagtacttcggccgaacagtgagcctcaagattcttgctgcaggtgtacatgttggccggcttgaggctacattgaggttgcctgctacaattaaaaaggttcaagaaattgagagtagatatagtggcaagttggtaatattaggtgtagatgacatggacatctttaaaggtatcagtctaaaactgcttggcttggagcttcttctggaaagaacacctaagcttcgaggcaaggttgtccttgtacagattgttaatcctgcaagaagcatcggaaaagacattgaggaagcgaaatatgaagctgaatctgtagctcaaaggataaatgataaatatggttctgctaattacaagcctgttgtcctcattgactactcaatacctttctatgaaaagatcgcattttatgctgcatctgactgctgtattgtaaatgctgtgagggatggcatgaatttgataccgtatgagtacaccgtatgcaggcagggaaatgaggagcttgataagctcagaggtcttaataagagctcatctcacacaagcacacttattgtgtctgaatttgtgggttgctctccatcacttagtggagcattcagggtaaatccttggagtatggaagatgtggctgatgcattatacagtgtaacagacctgacacgatatgagaagaatctgcgccatgaaaagcactatcgctatgtcaggtctcatgatgttgcttactgggcacgcagctttgaccaggatctggataaagcatgcatagagcaatacagccaaagatgttggacaactgggtttggtttaaattttagagttattgccctttcacctgggtttagaagactgtctctagaacacctagcctcgtcttataagaaggctaacaggaggatgatattcctggactacgatgggacccttgtgccgcagacatcacacgacaaatctccaagcgctgaacttatctctacccttaacagcttgtgcagtgatatgaagaacacagtatttatagtcagcggacgaggaagagattccctaagcgagtggtttgcttcatgcgagaacctcggtatcgctgccgaacatggttactttatcagatggaacaaagcagctgaatgggagacaagcttctcaggtatttattctgaatggaagctcatcgcggaccctatcatgcatgtatacatggaaacaactgatgggtccttcatagagccaaaagagagcgctttggtatggcactatcagaacacggatcatgactttggctcgtgccaggcaaaggagctagtgagccatcttgagcgagtcctatcgaacgaacctgttgtcgtgaggcgtggccatcagatcgtagaagttaaacctcagggagttagcaaggggatttccgtggacaagatcatccggaccttggtcagcaaaggggaagtaccagaccttttgatgtgcatcggaaacgatcggtcggacgaggacatgttcgagagcatcaacagagccacctccctttccgagcttcctgccgctccagaagtattcgcctgttccgttggccccaaggccagcaaggcaaactactacgtcgatggctgcgacgaagtaatcagactgctgaagggtgtcacagccgtttcgctccaaaaggatactgccggccatagccatgcggcattcgaggatacgcttgaggttgtcagctga

SEQ ID NO: 68 - protein, 864 aa, Zea mays
MVLKSHTNLLDMCCEDVFDFQQPLRSPRHVVNSPGIISDPDWESSNDGNSVGSMPFCFKRKIIVANFLPVICAKNEATGEWSFAMDDNQLLVQLKDGFPIGNEVIYVGSLNVQVDPIEQDRVSQKLFKEHRCVPTFLPAELQHQFYHIFCKQHLWPLFHYMLPVCHDKDELFDRSLFQAYVRANKIFADKIVEAVNSDDDCVWVHDYHLMLIPTLLRKKLHRIKVGFFLHSPFPSSEIYRTLPVRDEILKSLLNADLIGFQTFDYARHFLSCCSRLLGLNYESKRGHIGIEYFGRTVSLKILAAGVHVGRLEATLRLPATIKKVQEIESRYSGKLVILGVDDMDIFKGISLKLLGLELLLERTPKLRGKVVLVQIVNPARSIGKDIEEAKYEAESVAQRINDKYGSANYKPVVLIDYSIPFYEKIAFYAASDCCIVNAVRDGMNLIPYEYTVCRQGNEELDKLRGLNKSSSHTSTLIVSEFVGCSPSLSGAFRVNPWSMEDVADALYSVTDLTRYEKNLRHEKHYRYVRSHDVAYWARSFDQDLDKACIEQYSQRCWTTGFGLNFRVIALSPGFRRLSLEHLASSYKKANRRMIFLDYDGTLVPQTSHDKSPSAELISTLNSLCSDMKNTVFIVSGRGRDSLSEWFASCENLGIAAEHGYFIRWNKAAEWETSFSGIYSEWKLIADPIMHVYMETTDGSFIEPKESALVWHYQNTDHDFGSCQAKELVSHLERVLSNEPVVVRRGHQIVEVKPQGVSKGISVDKIIRTLVSKGEVPDLLMCIGNDRSDEDMFESINRATSLSELPAAPEVFACSVGPKASKANYYVDGCDEVIRLLKGVTAVSLQKDTAGHSHAAFEDTLEVVS

SEQ ID NO: 69 - DNA, 1008 bp, Zea mays
taactcatatccggttagataccaactacacatattgaatagcataaatctaataaatatatggcgcaatgaaaatagtaaataattaaatatgagtaaataatatgatgacaataatgaataatattggaacatgtacattgaccctattttgctaatatatacttattatatttgcttaatttggtaggatgtatatgtgattgaggcgggtataaattatccataggtatgtgggtataaatagtctatacttatacccatactcatatacccgacgggtatatgattgtgtccattgccatatctgcgggtaaaaaactcattatatacttgtccttataagtaaaacctgttggacactagagtttaggtaccatataattattaattttgaacgaaggaagtaatttgcagcgtattaaggtgcttctggtctagaagaaatgtcacaatgtttggtgttagtttttggtgaaatttaaggttaattactttttgaaagatgtttccactaggtggaaccgaaagaaacggtgccaaacacaccttacaacaagaaaatatttgtaaaaaaattattttgaataagatgtctaaaaatagaaagcgtgtatactttaggacggaggaatacatatgtatgattgggaaaaccgaaaacgtacacctcctcgctgcaatacgctggtgacttggcagttcgatcgcacccagcggataaagatgagcacggagaactcacaaggcacagccgcacaggcaggcaccagcgcgaacgcatggacgggcggcccctgagacgtgccgcccagctggcccgctgcgcccacacgtggcgcggagctgcgcgcggctcggccacgttataagccacgcgcgctggccgtcgccgcacctcctgactactgcacactcgtctccgcagtttgaaacgaagcccgcggctactgcaagctactccgtctccgtagctaaaggagaggtaggtttttatttggcgacgac

SEQ ID NO: 70 - DNA, 946 bp, Zea mays
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatc

SEQ ID NO: 71 - DNA, 1136 bp, Zea mays
aagcttgctactttctttccttaatgttgatttcccctttgttagatgttctttgtgttatatacactctgtatacaaggatgcgatacacacatcagctagtcctaatgatgccaccgactttacttgaggaaaaggaaacaaatatgatgtggccatcacattctcaataacaatgaccatgtgcgcaatgacataccatcatatttgatatcataaaaataaatttattatcaaagtaaacatatagttcatatatcagatattaaagtgataagaacaaatattacattttatcttatataaaatgacgaaaggtacgagttgaaaaggggtccaaccccttttttatagcttgttcggttgcttgttctccttcggctagcgaggtggtagaatgtgagagtgttgcgcgtggattcccgtcgtagtgttcttaggtgatttctcacggcccatctgtgatatagcgactcattatgtggtgtaatagcccattgggagaaggggagagatatagatctacgtgatttgcgcgtgatgcacgacgaacgaaactggtggtttaaagtagtagaggtttgtcattagtggtgtaagtggtacatatattatccgttcatattcgaatttgatccgtataagggggctaagatctaatccgtatacaagtccaagtattaagtatccgatccatatcggatctttatccgtatccgtatactcaaaatttgatgtttaagattttaatatatatttaaactttataggaactcgataatatttgtatctgatttgaattgtgaaaacaaatatggaacgattaatttcagtctatatccgttccgatatttgtcatgctttgctaaaaatacctttacaaggcatcttgtgcagattatatattaatctgaaatcagttagagaagcctacaaatttgaccaaatgccgagtcatccggcttatcccctttccaactttcagttctgcaagcgccagaaatcgtttttcatctacattgtctttgttgcctgcatacatctataaataggacctgctagatcaatcgcagtccatcggcctcagtcgcacatatctactatactatactctaggaagcaaggacaccaccgcc

SEQ ID NO: 72 - protein, 909 aa, Artificial Sequence
  Consensus sequence for Figure 1
MVSRSXANXLDLASXDXLXFPXXXXXXXXXXXXXXXXXXXXXRXLPRVMTVPGIISELDXXXXXXXXSSDVXSXXXXSRERKIIVANMLPLQAKRDXETXXXWXFSWDEDSLLLQLRDGXFSXDTEXLYVGSLXXDIXXSEQXXEEVSQKLLEEFNCVPTFLPXELQEKFYXGFCKHHLWPLFHYMLPMXPDHGXXXXDRFDRXLWQAYVSANKIFSDRVMEVINPEDDYVWIHDYHLMVLPTFLRKRFNRIKLGFFLHSPFPSSEIYRTLPVRDELLRGLLNCDLIGFHTFDYARHFLSCCSRMLGLDYESKRGHIGLDYFGRTVXIKILPVGIHMGRLESVLXLPXTAAKVKEIXEQFKGKKXXLILGVDDMDIFKGISLKLIAMEXLLETYXXLRGKVVLVQIVNPARSSGKDVEEVKKETYXTXKRINERYGSXGXYXPVVLIDRXVPRXEKTAYYAVADCCLVNAVRDGMNLVPYKYIVCRQGTXXLDXXXGIXXXSXRXSMLVVSEFIGCSPSLSGAIRVNPWDVDAVAEAMNXALXMSEXEKQLRHEKHYRYVSTHDVGYWAKSFMQDLERACRDHYXKRCWGIGFGLGFRVVSLXPSFRKLSIEHIVXXYRKTXRRAIFLDYDGTLVPXXXSSIXKTPSXEVISVLXALCXDPXNTVFIVSGRGRESLSXWLSPXCENLGIAAEHGYFIRWXXXXEWETCXXXADXEWKXMVEPVMRXYMEATDGSXIEXKESALVWHHQDADPDFGSCQAKELLDHLESXVLANEPVXVKRGQHIVEVKPQGVSKGLAAEKVIXXMXEXXGXPPDFVLCIGDDRSDEDMFESILSTVTXPXLXXXXEIFACTVGXXRKPSKAKYFLDDXADVLKLLXGLAXASSSXKPXXXXXXSXXXTQVAXEXXXXXX

SEQ ID NO: 73 - DNA, 153 bp, Silene pratensis
atggcttctacactctctaccctctcggtgagcgcatcgttgttgccaaagcaacaaccgatggtcgcctcatcgctaccaactaatatgggtcaagccttgtttggactgaaagccggttctcgtggcagagtgactgcaatggccacctac

SEQ ID NO: 74 - DNA, 519 bp, Arabidopsis thaliana
ttagatctcgtgccgtcgtgcgacgttgttttccggtacgtttattcctgttgattccttctctgtctctctcgattcactgctacttctgtttggattcctttcgcgcgatctctggatccgtgcgttattcattggctcgtcgttttcagatctgttgcgtttcttctgttttctgttatgagtggatgcgttttcttgtgattcgcttgtttgtaatgctggatctgtatctgcgtcgtgggaattcaaagtgatagtagttgatattttttccagatcaggcatgttctcgtataatcaggtctaatggttgatgattctgcggaattatagatctaagatcttgattgatttagatttgaggatatgaatgagattcgtaggtccacaaaggtcttgttatctctgctgctagatagatgattatccaattgcgtttcgtagttatttttatggattcaaggaattgcgtgtaattgagagttttactctgttttgtgaacaggcttgatcaaa

SEQ ID NO: 75 - DNA, 847 bp, Arabidopsis thaliana
tggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgtattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 76 - DNA, 822 bp, Oryza sativa
ggttcttggtatatgccaacttttgtagcctgcaccagaaacaaaaatgaagacttttgctaaagatgtaaaagtggcatgatgtcctggatgaccaaataattcatgacaaatggattaaaagagcccaatatctgaaagagactggccagcagccactaatgtcaccaaccacatatgtaacacttggtgcataattcaagagggagcatctcctccagaatcaggattgaaaggtacaacctcatagtaaatcctcggaatatagcatgtgcagcataagaatatatcagtgttgtgctgggtaagaaaccacatgaaccaattaggaataaataatcatgctgaaattatagcaatgcttgcaatttgcaaacgataaagctagacgcgggttgctggaataacaatccatctccaacaaaatagtacagaatataactgaatggccagctcagaccctaacagaattgaaaagctggattcatcagcactccattgagcaatctagatcaggaaagagcatagatgcataatgaactgagatcccttcaaaatgactaactaatatttttttttcttataaaagagtttacaacagtacaaccacgaagatcagcactaccattactgattttgttaacatagagtgatttatcatgtgtgccagacaaacaacagatacattcatacatagcataacttacagcacatgatacagactacggagaacggttaatcttaaaataaaaacaaaaaaacaaggaggcaaagcttattttgcctgggattcatctaaatgcagttgtgtgcagaaggaga

SEQ ID NO: 77 - DNA, 1198 bp, Zea mays
tcccgtgtccgtcaatgtgatactactagcatagtactagtaccatgcatacacacagcaggtcggccgcctggatggatcgatgatgatactacatcatcctgtcatccatccaggcgatctagaaggggcgtggctagctagcaaactgtgaccggtttttctacgccgataataatactttgtcatggtacagacgtacagtactggttatatatatctgtagatttcaactgaaaagctaggatagctagattaattcctgagaaacacagataaaattcgagcttggctatagatgacaaaacggaagacgcatgcattggacgacgtatgcaatgcgagcgcgtctcgtgtcgtcccgtccaagtctggcgatctcacgccacgtgctcaacagctcaaggactgttcgtcaccagcgttaaattcattgaagggatgacgcatttcggcatttgtcattgcttgtagctatatatatatatccaacagatttctctcaagcttttgtatgcgtgaatgtaaagtctagcttatacgacagcacgtgcagatatattaacgtcattattaggtggagagcaagatgcatgatctggtagaaattgtcgaaaacacaagagagagtgaagtgcacacttctggtataggagtgtatacgccgctggttggtgggcaatgcgcgccgcaatattggccaatgaaacctagcaacgcccactcgccacgccccatgaatggcccccgcacggcagcgagccagccagtgcccgcgcgcggcccagccggagtcggcggaacgcgccacgggggacgaggcgcccgagggccgaggcagcgcggcatggcaagcaagccgaagcgggcaagcgacctgcatgcagcccctgcccctcgccctcgtcagtcgtcccagcctcccactggaatccacccaacccgcccttcctctccaaagcacgcgccccgcgactcgcctccgcctacgtgtcggcagcgtccccgccggtcgcccacgtaccccgccccgttctcccacgtgcccctccctctgcgcgcgtccgattggctgacccgcccttcttaagccgcgccagcctcctgtccgggccccaacgccgtgctccgtcgtcgtctccgcccccagagtgatcgagcccactgacctggcccccgagcctcagctcgtgagtcc